(19) United States
(12) Patent Application Publication
(10) Pub. No.: US 2024/0065988 A1
(43) Pub. Date: Feb. 29, 2024

(54) COMBINED USE OF KETAMINE AND RETIGABINE (EZOGABINE) FOR THE TREATMENT OF PSYCHIATRIC DISORDERS

(71) Applicants: Yeda Research and Development Co. Ltd., Rehovot (IL); Max-Planck-Gesellschaft zur Förderung der Wissenschaften e.V., Munich (DE)

(72) Inventors: Alon CHEN, Rehovot (IL); Juan Pablo LOPEZ, Munich (DE)

(73) Assignees: Yeda Research and Development Co. Ltd., Rehovot (IL); Max-Planck-Gesellschaft zur Förderung der Wissenschaften e.V., Munich (DE)

(21) Appl. No.: 18/376,875

(22) Filed: Oct. 5, 2023

Related U.S. Application Data
(63) Continuation of application No. PCT/IL2022/050367, filed on Apr. 8, 2022.

(30) Foreign Application Priority Data
Apr. 8, 2021 (IL) 282188

(51) Int. Cl. A61K 31/135; A61K 31/216; A61P 25/24

(52) U.S. Cl. CPC A61K 31/135; A61K 31/216; A61P 25/24

(57) ABSTRACT

A method of treating a psychiatric disorder in a subject in need thereof is disclosed. The method comprising administering to the subject a therapeutically effective amount of an N-methyl-D-aspartate (NMDA) receptor antagonist and a therapeutically effective amount of a KCNQ channel activator. Pharmaceutical compositions comprising an NMDA receptor antagonist and a KCNQ channel activator are also disclosed.

### RELATED APPLICATIONS

This application is a Continuation of PCT Patent Application No. PCT/IL2022/050367 having International filing date of Apr. 8, 2022, which claims the benefit of priority of Israel Patent Application No. 282188 filed on Apr. 8, 2021. The contents of the above applications are all incorporated by reference as if fully set forth herein in their entirety.

### SEQUENCE LISTING STATEMENT

The XML file, entitled 98168SequenceListing.xml, created on Oct. 5, 2023, comprising 48,616 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to a combination therapy comprising an NMDA receptor antagonist and a KCNQ channel activator for the treatment of psychiatric disorders, more particularly, but not exclusively, to combined therapeutic use of ketamine and retigabine.

The discovery that a single sub-anesthetic dose of ketamine, a glutamate N-methyl-D-aspartate (NMDA) receptor blocker, can produce a rapid (i.e. within hours) antidepressant response that is sustained (i.e. typically lasting up to 7 days), even in treatment-resistant patients, has been considered one of the most profound breakthroughs in the field of depression in over 60 years since the development of first-generation antidepressants in the 1950's. Ketamine has proven effective for the treatment of suicidal ideation in emergency room settings and its fast-acting antidepressant effects have been demonstrated in many relevant human and animal studies. However, ketamine's routine clinical use for the treatment of depression is still restricted due to its dissociative effects, impact on sensory perception, as well as its addictive potential. These limitations have led investigators to further explore the exact mechanisms of action underlying ketamine's antidepressant clinical responses in an effort to understand its primary targets, which can ultimately lead to the development of novel and more specialized treatment interventions for depression. These treatments are intended to mimic, enhance or potentiate the unique antidepressant actions of ketamine but without its undesirable side effects.

Previous studies have indicated the ventral hippocampus (vHipp) to be an important site for ketamine and antidepressant action in the brain at the electro-chemical, molecular, cellular, and circuit levels. Studies investigating the molecular mechanisms underlying the fast-acting antidepressant effects of ketamine have mentioned activation of brain-derived neurotrophic factor (BDNF), tropomyosin receptor kinase B (TrkB), extracellular signal-regulated kinases (ERK1/2), mammalian target of rapamycin complex 1 (mTORC1), as well as neuroinflammatory and Ca2+ signaling, among others. However, the exact mechanism of action is still not fully understood. It is possible that some of the more elusive molecular components of this mechanism remain unclear due to important methodological limitations from these studies, specifically the absence of cell-type-specific information [A. Gururajan et al., Stress (2018) 21: 384-388]. As previous gene expression studies are limited to providing data from brain homogenates that average out the signature of thousands of distinct cells, one can expect that any signature of treatment response that is specific to a particular cell type has been diluted, masked or even distorted in these studies [A. Gururajan et al., Stress (2018), supra].

The KCNQ gene family is composed of five members (Kcnq1-5) but Kcnq2 and Kcnq3 are the dominant variants in the central nervous system and in combination generate a signature M-current, which regulates the overall neuronal excitability in the brain. Recent studies have highlighted a potential role of the KCNQ channel in the pathophysiology of stress-related disorders. For example, it has been shown that stress exposure modulates the expression of Kcnq2 and Kcnq3 in the medial prefrontal cortex (mPFC) [A. F. T. Arnsten et al., Neurobiol Stress (2019) 11: 100187] and the hippocampus of mice [C. Li et al., Neuroscience (2014) 280: 19-30]. In addition, Kcnq3 was found to be upregulated in the ventral tegmental area (VTA) of mice that are resilient to chronic stress [V. Krishnan et al., Cell (2007) 131: 391-404]. In a follow up study, Friedman and colleagues further demonstrated that overexpression of Kcnq3 in the VTA increases resilience to stress [A. K. Friedman et al., Nat Commun (2016) 7: 11671].

Retigabine, a KCNQ activator, has been previously shown to normalize neuronal hyperactivity and depressive-like behaviors in mice (8-day, i.p injections) [A. K. Friedman et al., (2016), supra]. Feng et al. have shown that neuroinflammation produced by stress exposure leads to overproduction and release of inflammatory cytokines, which ultimately increases neuronal excitability. According to Feng, these effects can be reversed using daily i.p. injections of retigabine [M. Feng et al., Neuroscience (2019) 406: 109-125]. In 2020, a small open label clinical trial assessed the antidepressant effects of retigabine in human patients diagnosed with major depressive disorder (MDD) and showed that chronic treatment (10 weeks) was associated with an improvement in depressive symptoms [A. Tan et al., Mol Psychiatry (2020) 25: 1323-1333]. These findings were recently replicated in a small randomized placebo-controlled trial testing the effect of retigabine on clinical outcomes in depressed patients [S. Costi et al., Am J Psychiatry, appiajp202020050653 (2021)]. Notably, the authors from both of these studies reported that retigabine was well tolerated, and no serious adverse events occurred during the trials.

Additional background art includes U.S. Patent Application nos. 20100297181 and 20200038420 both of which provide methods of treatment of mood disorders, including depression, using compositions comprising AMPA receptor antagonist or aminosterol, respectively, along with additional agents such as ketamine or retigabine.

U.S. Patent Application no. 20120232025 provides drug combinations for reduction of neurotoxic brain damage or psychotomimetic stresses due to sustained administration of NMDA antagonist, such as ketamine.

### SUMMARY OF THE INVENTION

According to an aspect of some embodiments of the present invention there is provided a method of treating a psychiatric disorder in a subject in need thereof, the method comprising administering to the subject a therapeutically effective amount of an N-methyl-D-aspartate (NMDA) receptor antagonist and a therapeutically effective amount of a KCNQ channel activator, thereby treating the subject.

According to an aspect of some embodiments of the present invention there is provided a therapeutically effective amount of an NMDA receptor antagonist and a therapeutically effective amount of a KCNQ channel activator for use in treating a psychiatric disorder in a subject in need thereof.

According to an aspect of some embodiments of the present invention there is provided a pharmaceutical composition comprising an NMDA receptor antagonist and a KCNQ channel activator, and a pharmaceutically acceptable carrier.

According to an aspect of some embodiments of the present invention there is provided an article of manufacture comprising an NMDA receptor antagonist and a KCNQ channel activator.

According to some embodiments of the invention, the NMDA receptor antagonist is selected from the group consisting of ketamine, Traxoprodil (CP-101606), MK-0657, Lanicemine (AZD6765), AVP-786, nitrous oxide, memantine, D-cycloserine (DCS), rapastinel (GLYX-13), and 4-chlorokynurenine (4-C1-KYNA) (AV-101) or analogs or derivatives thereof.

According to some embodiments of the invention, the NMDA receptor antagonist is a ketamine or analogs or derivatives thereof.

According to some embodiments of the invention, the therapeutically effective amount of the ketamine comprises a dose of 0.1-1.0 mg/kg body weight.

According to some embodiments of the invention, the therapeutically effective amount of ketamine comprises a dose of 0.1-1.0 mg/kg body weight for intravenous or intramascular route of administration.

According to some embodiments of the invention, the therapeutically effective amount of said ketamine comprises a dose of 10-300 mg for intranasal route of administration.

According to some embodiments of the invention, the therapeutically effective amount of said ketamine comprises a dose of 10-500 mg for oral route of administration.

According to some embodiments of the invention, the therapeutically effective amount of the ketamine is lower than the Gold standard administered to psychiatric patients.

According to some embodiments of the invention, the KCNQ channel comprises a Kv7.2 subunit.

According to some embodiments of the invention, the KCNQ channel activator is selected from the group consisting of retigabine (ezogabine), flupirtine, acrylamide (S)-1, acrylamide (S)-2, BMS-204352, ML213, NS15370, AaTXKp(2-6₄), diclofenac, meclofenamic acid, meclofenac, NH6, NH29, ICA-27243, ICA-069673, ICA-105665, N-ethylmaleimide, zinc pyrithione and hydrogen peroxide, or analogs or derivatives thereof.

According to some embodiments of the invention, the KCNQ channel activator is a retigabine (ezogabine), or analogs or derivatives thereof.

According to some embodiments of the invention, the therapeutically effective amount of the retigabine (ezogabine) comprises a dose of 0.5-2000 mg/day.

According to some embodiments of the invention, the therapeutically effective amount of the retigabine (ezogabine) comprises a dose of 0.5-2000 mg/day administered orally.

According to some embodiments of the invention, the KCNQ channel activator is ketamine and the KCNQ channel activator is a retigabine (ezogabine).

According to some embodiments of the invention, the ketamine and the retigabine (ezogabine) are to be administered concomitantly.

According to some embodiments of the invention, the ketamine is to be administered in a single dose.

According to some embodiments of the invention, the ketamine is to be administered in two or more doses.

According to some embodiments of the invention, the ketamine is to be administered by a mode of administration selected from the group consisting of an intranasal, an inhalation, an intravenous, an intramuscular, a subcutaneous, an oral, a sublingual, a transmucosal, a transdermal mode of administration.

According to some embodiments of the invention, the ketamine is to be administered intranasally.

According to some embodiments of the invention, the ketamine is to be administered intravenously.

According to some embodiments of the invention, the ketamine is to be administered orally.

According to some embodiments of the invention, the ketamine is to be administered intramuscularly.

According to some embodiments of the invention, the retigabine (ezogabine) is to be administered in a single dose.

According to some embodiments of the invention, the retigabine (ezogabine) is to be administered in two or more doses.

According to some embodiments of the invention, the retigabine (ezogabine) is to be administered by a mode of administration selected from the group consisting of an oral, an inhalation, an intranasal, a local injection, and an intravenous mode of administration.

According to some embodiments of the invention, the retigabine (ezogabine) is to be administered orally.

According to some embodiments of the invention, the NMDA receptor antagonist and the KCNQ channel activator are in a co-formulation.

According to some embodiments of the invention, the NMDA receptor antagonist and the KCNQ channel activator are in separate formulations.

According to some embodiments of the invention, the psychiatric disorder is a depression-related disorder.

According to some embodiments of the invention, the depression-related disorder is selected from the group consisting of a severe depression, a major depressive disorder (MDD), a treatment-resistant depression, a postpartum depression and a psychotic depression.

According to some embodiments of the invention, the psychiatric disorder is selected from the group consisting of a bipolar disorder, a schizophrenia, a neuropathic pain, a post-traumatic stress disorder (PTSD), an obsessive-compulsive disorder (OCD), a pervasive developmental disorder (PDD), a post-traumatic stress disorder (PTSD), a panic attack, an anxiety disorder, a social phobia, a sleep disorder, an eating disorder, a stress, a fatigue, a chronic pain and a substance-related disorder.

According to some embodiments of the invention, the subject is a human being.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F illustrate a cell-type specific transcriptomic characterization of the ventral hippocampus after ketamine treatment. (FIG. 1A) Experimental timeline. CD-1 mice were injected with ketamine (10 mg/kg/BW) or a saline solution and were sacrificed two days post-injection. Brains were collected and individual cell suspensions were prepared from the ventral hippocampus of ketamine-treated (n=4) and saline-treated controls (n=4). (FIG. 1B) UMAP plot depicting single cells from the ventral hippocampus (vHipp). Colors represent each of the 13 Louvain clusters of individual cell-types identified as: glutamatergic neurons (nGlut), GABAergic neurons (nGABA), oligodendrocytes, oligodendrocyte progenitor cells (OPCs), astrocytes, endothelial, microglia, macrophages, ependymal, pericytes, meningeal, vascular cells, and blood cells. (FIG. 1C) UMAP plot showing the distribution of cells by treatment: saline (grey) and ketamine (blue). (FIG. 1D) Number of differentially expressed genes (DEGs) in 7 clusters of the vHipp. (FIG. 1E) The Social Box (SB) arena. Each SB apparatus contained a large and small nest, an S-wall labyrinth, two ramps, as well as two feeders and two water bottles (proximal & distal) providing ad libitum access to food and water; (FIG. 1F) Experimental timeline. Groups of four CD-1 mice were housed together in a SB under continuous video observation for a total period of six days/nights. Mice were allowed to acclimatize to the SB environment for two nights, followed by two nights of baseline monitoring. Prior to the beginning of the dark phase on Day 5, all mice were removed from the SBs and injected with ketamine (10 mg/kg body weight (BW), n=3 per group) or a saline solution (n=1 per group). Thirty minutes after the injection, mice underwent the forced swim test (FST). Following a recovery period, all mice were returned to a clean SB at the start of the dark phase for response monitoring. Mice were sacrificed and tissues were collected immediately after the end of the dark phase on Day 6;

FIGS. 2A-C illustrate the validation of single-cell RNA seq data via FACS. (FIG. 2A) Nex-Cre-Ai9 mutant mice, where all glutamatergic neurons of the hippocampus are fluorescently labeled (tdTomato), were injected with ketamine (10 mg/kg BW) (n=4) or a saline solution (n=4). Following a recovery period of 36 hours, individual cell suspensions were prepared from the ventral hippocampus of all mice. Glutamatergic neurons (Ai9+) and all remaining cell-types (Ai9-) from the ventral hippocampus were isolated using fluorescence-activated cell sorting (FACS); (FIGS. 2B-C) Box plots represent qPCR mRNA levels of 8 (4 up-regulated and 4 down-regulated) of the 165 DEGs in glutamatergic neurons (scRNA-seq analysis) in Ai9+ and Ai9—cells between ketamine- and saline-treated mice. Saline (grey), ketamine (blue). All qPCR data was normalized to the combined mRNA expression of the endogenous controls, Gapdh and Rp113. One-way-ANOVA, corrected for multiple comparisons, ****p<0.0001, ***p<0.001, **p<0.01, *p<0.05, ‡p<0.1.

FIGS. 3A-N illustrate the effects of ketamine and HNKet stimulation in vitro and ex-vivo. (FIGS. 3A-H) Primary hippocampal neurons treated with either ketamine (10 μM), hydroxynorketamine (HNKet) (10 μM), or saline vehicle control for 2, 12, 24 or 48 hours. Bar plots represent qPCR mRNA expression levels of 8 selected genes (4 down-regulated and 4 up-regulated) from scRNA-seq analysis at different time points. All qPCR data was log 2 normalized to the geometric mean of Gapdh and Rp113. One-way ANOVA, corrected for multiple comparisons; (FIGS. 3I-N) Electrophysiological analysis of I_{M} tail current density in primary hippocampal neurons and CA1 pyramidal cells from the ventral hippocampus (vHipp) in acute brain slices. (FIG. 31) Primary cultures were treated for 24 hours with saline vehicle (grey) or HNKet (10 μM, orange-red), (FIG. 3L) vHipp slices were obtained from CD-1 mice that received an injection (i.p) of saline vehicle (grey) or ketamine (10 mg/kg BW) (blue), 36 hours before slice preparation, (FIGS. 3J, 3M) Representative current traces from whole-cell voltage-clamp recordings before and after application of the selective IM inhibitor XE991 (40 μM); (FIGS. 3K, 3N) Quantification of I_{M} tail current density for individual cells. In vitro (n=8 cells for each group from 2 independent cultures, grey=saline; orange=HNKet). Ex vivo (grey: n=5, 10 cells; blue: n=6, 12 cells). Unpaired t-tests, two-tailed. ****p<0.0001, ***p<0.001, **p<0.01, *p<0.05.

FIGS. 4A-G illustrate that in vivo manipulation of Kcnq2 in the mouse ventral hippocampus modulates antidepressant-like behaviors. (FIG. 4A) Schematic overview of the Kcnq2 knockdown (green) and control (grey) construct with eGFP and shRNA driven by EFla and H1 promoter, respectively. (FIGS. 4B-C) Representative images of N2a cells transfected with an shRNA-control, or two different shRNA-Kcnq2 vectors. (FIG. 4B) Fluorescent eGFP signal (green); DAPI signal (blue). (FIG. 4C) Box plots represent qPCR mRNA expression levels of Kcnq2 after shRNA-Kcnq2 knockdown compared to a shRNA control. One-way-ANOVA, corrected for multiple comparisons (FIGS. 4D-E) Coronal map with the region and Bregma coordinates (mm) targeted for in vivo viral manipulation. AP (Antero-posterior), ML (Medio-lateral), DV (Dorso-ventral). (FIG. 4F) Representative images of mouse brains injected with shRNA-Kcnq2 and shRNA-control AAV virus, respectively. Fluorescent eGFP signal (green); DAPI signal (blue). CD-1 mice were injected in the ventral hippocampus (bilateral) with either shRNA-Kcnq2 (n=22) or shRNA-control (n=19) AAV virus. Four weeks after viral injection, half of the mice were randomly selected to receive a ketamine (10 mg/kg/BW) or saline injection. (FIG. 4G) Box plots represent total immobile time (seconds) during the forced swim test (FST) in mice who received shRNA-Ctrl (grey) or shRNA-Kcnq2 (green) after ketamine (dark green) or saline injection (dark grey). The FST was performed 2 days after treatment. Two-way-ANOVA. Multiple testing correction was performed using the Benjamini-Hochberg method *p<0.05.

FIGS. 5A-F illustrate that chronic stress exposure and ketamine modulate Kcnq2 mRNA in the ventral hippocampus. (FIG. 5A) Experimental timeline of chronic social defeat stress (CSDS) paradigm for control (n=5) and stressed (n=5) mice. (FIG. 5B) Box plots represent total immobile time (seconds) during the forced swim test (FST) in non-stressed controls (n=6, grey) and CSDS mice (n=6, purple). The FST was performed 1 day after the last social defeat session. Unpaired t-test, two tailed. (FIG. 5C) Box plots represent qPCR mRNA levels of Kcnq2 in tdTomato+ and tdTomato-cells between non-stressed controls (n=4, grey) and CSDS mice (n=4, purple). One-way-ANOVA. Multiple testing correction was performed using the Benjamini-Hochberg method. (FIG. 5D) Experimental timeline of CSDS paradigm plus treatment. (FIG. 5E) Box plots represent total immobile time (seconds) during the FST in chronically stressed mice who received a saline (n=6, purple) or ketamine (n=6, blue) treatment. The FST was performed 2 days after treatment. Unpaired t-test, two tailed. (FIG. 5F) Box plots represent qPCR mRNA levels of Kcnq2 in tdTomato+ and tdTomato-cells between chronically stressed mice who received a saline (n=4, purple) or ketamine (n=4, blue) treatment. One-way-ANOVA. Multiple testing correction was performed using the Benjamini-Hochberg method. ***p<0.001, **p<0.01.

FIGS. 6A-E illustrate that ketamine regulates Kcnq2 via calcium/calmodulin and Akap5 signaling. (FIGS. 6A-D) Hippocampal primary neurons (mouse) stimulated with either saline solution, hydroxynorketamine (HNKet) (10 μM), or a combination of HNKet and nifedipine (calcium channel blocker), W-7 hydrochloride (calmodulin inhibitor), or cyclosporine-A (calcineurin inhibitor) for 30 minutes, 1, 2, or 6 hours, and compared to an untreated control. Box plots represent qPCR mRNA levels of Kcnq2 log 2 normalized to the geometric mean of the endogenous controls, Gapdh and Rp113. One-way-ANOVA, corrected for multiple comparisons, ****p<0.0001, ***p<0.001, *p<0.05; (FIG. 6E) Schematic model for transcriptional regulation of Kcnq2. Ketamine or its active metabolite, hydroxynorketamine (HNKet), increase L-type calcium channel (L-VDCC) activity creating elevated levels of intracellular calcium (Ca²⁺). (1) Known Pathway: The burst of intracellular Ca2'⁰ causes release of brain-derived neurotrophic factor (BDNF) and stimulation of Akt, Erkl/2, and mTORC1 signaling which causes rapid increases in synaptic protein synthesis and leads to antidepressants effects of ketamine. (2) Novel Pathways: (2.1) Direct regulation of KCNQ by calmodulin. The burst of intracellular Ca2'⁰ causes activation of calmodulin (CaM), a calcium-sensor. CaM directly binds to the C-terminal domains of KCNQ which leads to a fast activation of KCNQ activity, regulation of neuronal excitability and antidepressant effects. (2.2) Transcriptional regulation of Kcnq2 by Akap5-CaM-CaN complex. Ca²⁺ causes activation of CaM which binds to the A-kinase anchor protein 5 (AKAP5) and calcineurin (CaN). The transcription factor NFAT is activated by the AKAP5/CaM/CaN complex and dephosphorylated, which leads to translocation of NFAT to the nucleus, where it acts on Kcnq2 gene regulatory elements. Enhanced Kcnq2 transcription leads to regulation of neuronal excitability and antidepressant effects.

FIGS. 7A-I illustrate that pharmacological manipulation of KCNQ modulates antidepressant-like behaviors. (FIG. 7A) Schematic overview of pharmacological manipulation of KCNQ using XE991 (KCNQ inhibitor) or retigabine (KCNQ activator). Each mouse (C57BL/6N) was treated with saline, ketamine (10 mg/kg/BW) alone, or a combination of ketamine with XE991 (1 and 3 mg/kg/BW) or retigabine (1 and 5 mg/kg/BW). (FIGS. 7B-C) Box plots represent total immobile time (seconds) during the FST, two days after treatment. One-way-ANOVA, corrected for multiple comparisons. (FIG. 7D) The Social Box (SB) arena. Each SB apparatus contained a large and small nest, an S-wall labyrinth, two ramps, as well as two feeders and two water bottles (proximal & distal) providing ad libitum access to food and water. (FIG. 7E) Experimental timeline. Groups of four CD-1 mice were housed together in a SB under continuous video observation for a total period of six days/nights. Mice were allowed to acclimatize to the SB environment for two nights, followed by two nights of baseline monitoring. Prior to the beginning of the dark phase on Day 5, all mice were removed from the SBs and injected with ketamine (10 mg/kg/BW, n=3 per group) or a saline solution (n=1 per group). Following a recovery period, all mice were returned to a clean SB at the start of the dark phase for response monitoring. (FIGS. 7F-G) Behavioral outcomes from the SB were summarized as change from the mean over the Baseline days and used as input for partial least squares discriminant analysis (PLS-DA). (FIG. 7H) Experimental timeline. On day 5, mice were injected with saline, ketamine (10 mg/kg/BW), or ketamine in combination with retigabine (1 and 5 mg/kg/BW). (FIG. 71) Box plots represent response to ketamine (P-KET) in the SB. Behavioral outcomes were summarized as change from the mean over the baseline days and used as input for PLSD analysis. Conditions: saline (grey), saline-ketamine (dark blue), ketamine-retigabine (dark orange). One-way-ANOVA. Multiple testing correction was performed using the Benjamini-Hochberg method. ***p<0.001, **p<0.01, *p<0.05.

FIGS. 8A-G illustrate that adjunctive treatment with retigabine augments the antidepressant-like effects of ketamine but not escitalopram in mice. (FIG. 8A) Overview of treatment. Each mouse was injected with saline or ketamine (10 mg/kg/BW), in the absence or in combination with retigabine (1 mg/kg/BW). (FIGS. 8B-C) Box plots represent total immobile time (seconds) during the FST at days 5 and 7 post-injection. One-way-ANOVA. Multiple testing correction was performed using the Benjamini-Hochberg method. (FIG. 8D) Schematic overview of pharmacological manipulation. Each mouse was injected with saline, ketamine (1, 5 or 10 mg/kg/BW) in the absence or in combination with retigabine (1 mg/kg/BW). (FIG. 8E) Box plots represent total immobile time (seconds) during the FST (2 days post injection). One-way-ANOVA. Multiple testing correction was performed using the Benjamini-Hochberg method. (FIG. 8F) Overview of treatment. Each mouse was injected with saline, escitalopram (10 mg/kg/BW), or ketamine (10 mg/kg/BW), in the absence or in combination with retigabine (1 mg/kg/BW). (FIG. 8G) Box plots represent qPCR mRNA levels of Kcnq2 in tdTomato+ and tdTomato-cells. One-way-ANOVA. Multiple testing correction was performed using the Benjamini-Hochberg method. ****p<0.0001, ***p<0.001, **p<0.01.

FIG. 9A illustrates quality control (QC)—scRNA-seq data. Scatterplot of the count depth and number of genes expressed in each cell, color indicates the fraction of counts of mitochondrial genes. The red dashed lines show the thresholds that were used during QC steps. Cells with a count depth below 1750 and above 42000 were removed, as well as cells with counts belonging to less than 700 genes. Additionally, cells with a mitochondrial gene percentage above 20% were removed.

FIG. 9B illustrates the identity of cell clusters from the ventral hippocampus. Dot plots showing the expression of cell-type-specific markers. All neurons (Ndgr4, Syp, Rbfox3), glutamatergic neurons (Slc17a8, Gria2, Grin1), GABAergic neurons (Gabrg2, Gabral, Tac2), astrocytes (Gja1, Slcla2, Slcla3), oligodendrocytes (Mag, Mog, Plpl), oligodendrocyte progenitor cells (OPCs) (Pdgfra, Cspg4, Vcan), microglia (Ctss, Csfrl, P2ry12), macrophages (Pf4, Mrcl, Lyzl), endothelial cells (Ly6c1, Cldn5, Ly6c2), ependymal cells (Ccdc153, Mia, Dynlrb2), vascular cells (Myl9, Acta2, Tagln), pericytes (Higdlb, Vtn, Atpl3a5), meningeal cells (Apod, Dcn, Igf2), and blood cells (Hba-ps4, Hba-a1, Hba-a2). Size of the dot represents the percentage of cells within a cluster expressing a given marker gene. Color intensity (white to red) represents the average expression levels of a given marker gene within a particular cell cluster.

FIG. 10 illustrates an overlap of DEGs. Venn diagram shows all differentially expressed genes (DEGs) and the overlap between cell populations in the single-cell analysis. The Venn diagram was generated using InteractiVenn (Heberle et al., BMC Bioinformatics (2015) 16: 169).

FIGS. 11A-C illustrate a pathway enrichment analysis. (FIGS. 11A-C) Enrichment analysis (KEGG) for the three cell types with the largest DEGs (glutamatergic neurons, astrocytes and oligodendrocytes). Bar plots show the top 15 enriched pathways per cell type and ranked by adjusted p values. Significant pathways (p<0.05) are highlighted by cluster color: Glutamatergic neurons (blue), astrocytes (green) and oligodendrocytes (red). Non-significant clusters are shown in grey.

FIG. 12 illustrates Hippocampal Neurod6. Expression (in situ) of Neurod6 mRNA in the dorsal and ventral hippocampus of the mouse brain (age P56). Image was adapted from the Allen Brain Atlas data portal. Color scale: no expression (white), medium (light purple), high (dark purple). Neurod6 is expressed in pyramidal neurons of the hippocampus (CA1, CA2, CA3) but is missing in the dentate gyrus (DG).

FIGS. 13A-G illustrate a validation of Nex-Cre;Ai9 mutant mouse line by FACS. (FIGS. 13A-B) Contour plots represent the density of tdTomato (+) (inside of small box) and tdTomato (−) single-cells from the ventral hippocampus of wildtype and Nex-Cre;Ai9 mice, sorted by fluorescence-activated cell sorting (FACS). (FIG. 13C) Contour plot represents the density of re-sorted tdTomato+ and tdTomato-cells from Nex-Cre;Ai9 mice. (FIG. 13D) Box plots represent qPCR mRNA levels of tdTomato, Neurod6, and cell-type-specific marker genes present in tdTomato+(red) and tdTomato-(grey) cells. Slc17a7 (Glutamatergic Neurons), Slc32al (GABAergic Neurons), Slcla3 (astrocytes), Mog (oligodendrocytes), C1qc (microglia), and Cldn5 (endothelial cells). All qPCR data was normalized to the combined mRNA expression of the endogenous controls, Gapdh and Rp113. One-way-ANOVA. Multiple testing correction was performed using the Benjamini-Hochberg method. ****p<0.0001. (FIGS. 13E-F) Representative contour plots showing the density of tdTomato+ and tdTomato-single-cells from the ventral hippocampus of Nex-Cre;Ai9 mice treated with saline or ketamine (10 mg/kg/BW). (FIG. 13G) Box plot represents the percentage of tdTomato+ cells sorted in both experimental groups (saline vs ketamine). Unpaired t-tests, two-tailed. tdT=TdTomato.

FIGS. 14A-B illustrate bulk mRNA expression of Kcnq2 after ketamine treatment in the ventral hippocampus. (FIG. 14A) Tissue homogenates were obtained from the ventral hippocampus of mice that received an injection (i.p) of saline vehicle (grey) or ketamine (10 mg/kg/BW) (blue), 2 days before tissue collection. (FIG. 14B) Box plots represent “bulk” qPCR mRNA levels of Kcnq2 between ketamine- and saline-treated mice. All qPCR data was normalized to the combined mRNA expression of the endogenous controls, Gapdh and Rp113. One-way-ANOVA. Multiple testing correction was performed using the Benjamini-Hochberg method ‡p<0.1.

FIGS. 15A-G illustrate cell viability assays. (FIGS. 15A-F) Mouse hippocampal neurons treated with ketamine or hydroxynorketamine (HNKet) at various concentrations (10 nM, 100 nM, 1 μM, 10 μM, 100 μM and 1 mM) and timepoints (1, 2, and 6 hours). CellTiter-Glo® Reagent (Promega) was added into cells after incubation. Luminescence was detected with a luminometer and used as a readout of cell viability. Experiments were run in triplicates in order to improve the reproducibility of the assay. Bar plots represent Luminescence (RLU) after incubation. One-way ANOVA. Multiple testing correction was performed using the Benjamini-Hochberg method. (FIG. 15G). Dose Response Experiment: Primary hippocampal neurons treated with a single dose of either ketamine or HNKet at two different concentrations (2 μM and 10 μM). Neurons were collected 24 hrs post-injection. Bar plots represent qPCR mRNA expression levels of Kcnq2. All qPCR data was Log2 transformed and normalized to the geometric mean of Gapdh and Rp113. One-way ANOVA. Multiple testing correction was performed using the Benjamini-Hochberg method.

FIGS. 16A-C illustrate a protocol to elicit IM current in CA1 pyramidal neurons of the ventral hippocampus and digital subtraction to obtain the KCNQ2/3 component of the total IM current. (FIGS. 16A-B) Example traces obtained from whole-cell patch-clamp recording from ventral pyramidal CA1 neurons from an acute slice from a vehicle-treated (FIG. 16A) and a ketamine-treated (FIG. 16B) animal. The voltage step protocol applied to the recorded cell is shown above the traces: −a 1 s step from the holding potential (−70 mV) to −10 mV was applied (to activate IM while inactivating most other voltage-gated currents); −a 1 s step to −50 mV (to elicit IM tail current); −a 0.5 s step to −10 mV, before returning to the holding potential. For each experimental group (vehicle and ketamine treatment), the cell was recorded under baseline conditions (black trace) for 5 min (stimulation protocol applied every 10 sec). Then, the effect of XE991 (a selective KCNQ2/3 antagonist) on IM was evaluated 10 min after bath application (red traces). (FIG. 16C) To isolate specifically the KCNQ2/3 component of the total IM and the effect of ketamine on it, a digital subtraction was performed with the offline analysis software (ClampFit, Molecular Device): KCNQ 2/3 current=(IM current under baseline)—(IM current after XE991). After isolation of the KCNQ2/3 component of the IM current for each recorded cell, the ITail current amplitude was measured in vehicle-treated (blue trace) and ketamine-treated (orange trace) mice. The ketamine effect on KCNQ2/3 channel is presented in FIGS. 31 to 3N as box plots comparing the KCNQ2/3 current density (in pA/pF) for both vehicle and ketamine-treated animals.

FIGS. 17A-F illustrate the effects of Ketamine and HNKet on Kcnq3. (FIG. 17A) Kcnq3 mRNA expression in glutamatergic neurons. Violin plots represent log-normalized scRNA-Seq data. Ketamine (10 mg/kg) (blue) (n=4). Saline solution (grey) (n=4). (FIG. 17B) Nex-Cre-Ai9 mutant mice. Glutamatergic neurons (tdTomato+) and all remaining cell-types (tdTomato-) from the ventral hippocampus isolated using fluorescence-activated cell sorting (FACS). Mice were injected with ketamine (10 mg/kg/BW) (blue) (n=4) or a saline solution (n=4) (gray). Box plots represent qPCR mRNA levels of Kcnq3. (FIG. 17C) Hippocampal primary neurons (mouse) treated with ketamine (10 μM) (blue), hydroxynorketamine (HNKet) (10 μM) (orange), or saline control (white). Bar plots represent qPCR mRNA expression levels of Kcnq3 at different time points. All qPCR data is normalized to the geometric mean of the endogenous controls, Gapdh and Rp113. One-way-ANOVA, corrected for multiple comparisons. (FIG. 17D) ISH log 2 expression values for Kcnq2 and Kcnq3 in the mouse brain. CTX=Isocortex; OLF=Olfactory areas; HPF=Hippocampal formation; CTXsp=Cortical subplate; STR=Striatum; PAL=Pallidum; TH=Thalamus; HY=Hypothalamus; MB=Midbrain; P=Pons; MY=Medulla; CB=Cerebellum. (FIGS. 17E-F) 3D heatmap expression (in situ) of Kcnq2 and Kcnq3. Image was adapted from the Allen Brain Atlas data portal. Color scale: no expression (white), low (green), medium (dark green), high (yellow), very high (red).

FIG. 18 illustrates cell-type specific mRNA expression of the mouse brain. Expression (mRNA) of Kcnq2 and Kcnq3 across different brain regions (top) and cell types (bottom). The heat map (top) indicates the strength of mRNA expression, with darker colors (blue) indicating stronger expression. Dot plots (bottom) indicate mRNA expression across hippocampal neurons, glia and vascular cells. Size of the dots indicates higher mRNA expression. Of note, Kcnq2 is mainly expressed in neurons, while Kcnq3 is expressed in neurons, astrocytes, oligodendrocytes and OPCs. Image was adapted from mousebrain.org.

FIGS. 19A-C illustrate cell-type specific mRNA expression of the developing mouse brain. (FIG. 19A) tSNE plot showing the major cell-types of the developing mouse brain (E10.5). (FIGS. 19B-C) Expression (mRNA) of Kcnq2 and Kcnq3 across different cell types. The heat map indicates the strength of mRNA expression, with darker colors (orange) indicating stronger expression. Kcnq2 is mainly expressed in neurons, while Kcnq3 is expressed in neurons, astrocytes, oligodendrocytes and OPCs. Image was adapted from mousebrain.org.

FIGS. 20A-C illustrate cell-type specific mRNA expression of the mouse whole cortex and hippocampus. (FIG. 20A) tSNE plot showing the major cell-types of the mouse whole cortex and hippocampus. (FIGS. 20B-C) Expression (mRNA) of Kcnq2 and Kcnq3 across different cell types. The heat map indicates the strength of mRNA expression, with darker colors (blue) indicating stronger expression. Kcnq2 is mainly expressed in neurons, while Kcnq3 is expressed in neurons, astrocytes, oligodendrocytes and OPCs. Image was adapted from the Allen Brain Map.

FIGS. 21A-C illustrate forced swim test (FST). (FIG. 21A) Box plots represent total immobile time (seconds) during the FST in mice who received shRNA-Ctrl (grey) or shRNA-Kcnq2 (green) after ketamine (dark green) or saline injection (dark grey). The FST was performed 30 minutes after treatment. Two-way-ANOVA. Multiple testing correction was performed using the Benjamini-Hochberg method *p<0.05. (FIGS. 21B-C) Home Cage Locomotion. Activity was measured as the number of beam breaks (5 mins) for a 60-min period following treatment (FIG. 21B) or 2 days post injection (FIG. 21C). Of note, there was no significant difference in the locomotion activity between groups (ketamine vs. saline) at any of the timepoints tested. Two-way-ANOVA. Multiple testing correction was performed using the Benjamini-Hochberg method.

FIGS. 22A-F illustrate chronic stress exposure. (FIGS. 22A-C) Ten days of social defeat exposure significantly increased (a.m.) basal corticosterone (CORT) levels, enhanced adrenal weight, and reduced fur quality in stressed mice. Coat state score: (0) no wounds, well-groomed and bright coat, and clean eyes; (1) no wounds, less groomed and shiny coat OR unclean eyes; (2) small wounds, AND/OR dull and dirty coat and not clear eyes; (3) extensive wounds, or broad piloerection, alopecia, or crusted eyes. (FIG. 22D) Body weight was significantly affected by stress only during the first week of the stress paradigm. (FIGS. 22E-F) Box plots represent qPCR mRNA levels of Kcnq2 in tissue homogenates “bulk tissue” between control and stressed mice (FIG. 22E) or ketamine- and saline-treated mice (FIG. 22F). Control (grey), stress (pink), stressed-saline (pink), stressed-ketamine (blue). All qPCR data was normalized to the combined mRNA expression of the endogenous controls, Gapdh and Rp113. Unpaired t-tests, two-tailed. ***p<0.001, *p<0.05.

FIGS. 23A-B illustrate that pharmacological manipulation of Kcnq2 modulates antidepressant-like behaviors. (FIGS. 23A-B) Pharmacological manipulation of KCNQ using XE991 (KCNQ inhibitor) or retigabine (KCNQ activator). Each mouse (C57BL/6N) was treated with XE991 (1 and 3 mg/kg/BW), retigabine (1 and 5 mg/kg/BW), or a saline control in the absence or in combination with ketamine (10 mg/kg/BW). Box plots represent total immobile time (seconds) during the FST. The FST was performed 30 minutes after treatment. One-way-ANOVA. Multiple testing correction was performed using the Benjamini-Hochberg method. ****p<0.0001, ***p<0.001, **p<0.01.

FIGS. 24A-D illustrate the characterization of the behavioral response to ketamine in the Social Box. (FIG. 24A) Loadings of behavioral readouts onto the PLSDA-based classifier of ketamine response in the Social Box. Larger loadings indicate increased relative importance of the readout, color indicates the direction of the loading. The sign of a loading indicates the direction of the contribution of the behavioral readout values to the inferred probability that an individual received ketamine rather than saline. Points next to the label of each readout specify whether the behavior relates to food/water, the open area, locomotion, or social (inclusive); (FIGS. 24B-D) Selected behavioral readouts with high contributions to the classifier. Density plot fill colors represent the ranges between different percentiles (5th, 25th, 50th, 75th, and 95th) while the vertical lines indicate the location of the percentiles. Exact values are depicted below the density plots as thin vertical lines.

FIGS. 25A-B illustrate the Social Box (SB). (FIG. 25A) The Social Box (SB) arena. (FIG. 25B) Mice were injected with saline, ketamine (10 mg/kg/BW), or ketamine in combination with retigabine (1 and 5 mg/kg/BW) or XE991 (1 mg/kg/BW). Box plots represent response to ketamine (P-KET) in the SB. Behavioral outcomes were summarized as change from the mean over the baseline days and used as input for PLSD analysis. Conditions: saline (grey), saline-ketamine (dark blue), ketamine-retigabine (dark orange), ketamine-XE991 (green). One-way-ANOVA. Multiple testing correction was performed using the Benjamini-Hochberg method. ***p<0.001, **p<0.01, *p<0.05.

FIGS. 26A-C illustrate that adjunctive treatment with retigabine augments the antidepressant-like effects of ketamine but not escitalopram in mice. Each mouse was injected with saline, ketamine (1, 5 or 10 mg/kg/BW), or escitalopram (1, 5 or 10 mg/kg/BW), in the absence or in combination with retigabine (1 mg/kg/BW). Box plots represent total immobile time (seconds) during the FST. The FST was performed 30 minutes after treatment (FIGS. 26A,B) or two days after treatment (FIG. 26C). One-way-ANOVA. Multiple testing correction was performed using the Benjamini-Hochberg method. ****p<0.0001, ***p<0.001, **p<0.01.

FIGS. 27A-B illustrate Kcnq2 mRNA and the fast-acting antidepressant effects of ketamine. (FIG. 27A) Overview of treatment. Each mouse was injected with saline, escitalopram (10 mg/kg/BW), or ketamine (10 mg/kg/BW), in the absence or in combination with retigabine (1 mg/kg/BW) (FIG. 27B) Box plots represent qPCR mRNA levels of Kcnq2 in tdTomato+ and tdTomato-cells. One-way-ANOVA. Multiple testing correction was performed using the Benjamini-Hochberg method.

### DESCRIPTION OF SPECIFIC EMBODIMENTS OF THE INVENTION

The present invention, in some embodiments thereof, relates to a combination therapy comprising an NMDA receptor antagonist and a KCNQ channel activator for the treatment of psychiatric disorders including depression and, more particularly, but not exclusively, to combined therapeutic use of ketamine and retigabine.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have uncovered the molecular mechanisms underlying the fast-acting antidepressant effects of ketamine. The present inventors have also uncovered means to increase the antidepressant effects of ketamine whilst further reducing the sub-anesthetic doses thereof needed to achieve an efficient antidepressant effect.

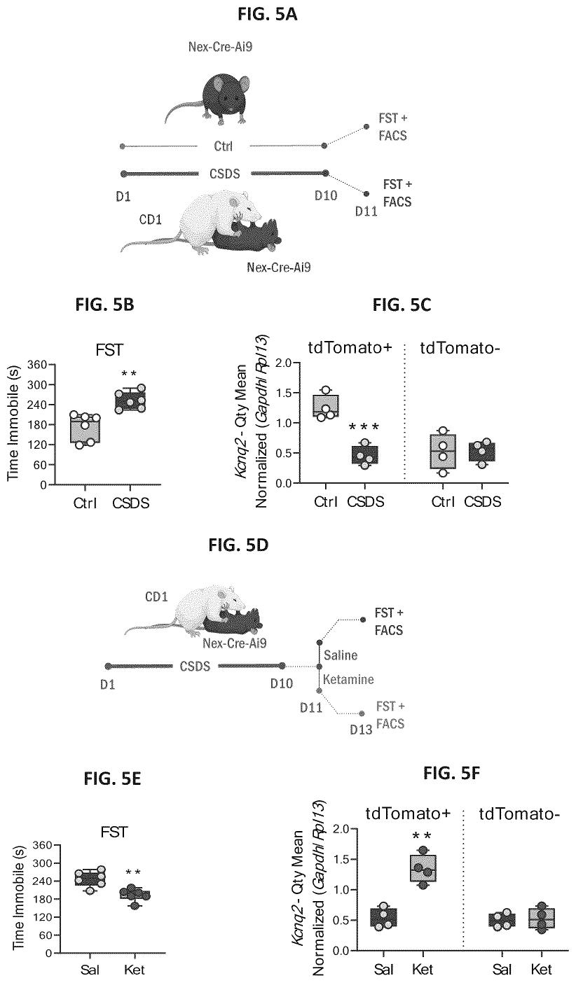

As is shown herein below and in the Examples section which follows, the present inventors have comprehensively cataloged the transcriptome of thousands of single cells from the ventral hippocampus (vHipp) of mice treated with a single dose of (R,S)-ketamine or a saline vehicle control, using single-cell RNA sequencing (scRNA-seq), and uncovered cell-type specific transcriptional signatures associated with the antidepressant effects of ketamine (see Example 1, herein below). Notably, the present inventors identified the Kcnq2 gene as an important target of ketamine action in glutamatergic neurons of the vHipp (see Examples 1-3, herein below). These findings were validated using glutamatergic neurons sorted from a conditional mouse reporter line, in vitro primary hippocampal neurons, electrophysiological recordings in vitro and from acute vHipp slices (see Example 3, herein below), as well as a viral-mediated knockdown of Kcnq2 in the vHipp of mice (see Example 4, herein below). The present inventors illustrated that Kcnq2 mRNA is altered after chronic stress exposure in glutamatergic neurons of the vHipp and that these effects can be reversed by ketamine treatment (see Example 5, herein below). In addition, the present inventors identified a previously unknown mechanism of action for ketamine via Kcnq2 in glutamatergic neurons of the hippocampus (see Example 5 herein below), as summarized in FIG. 5E.

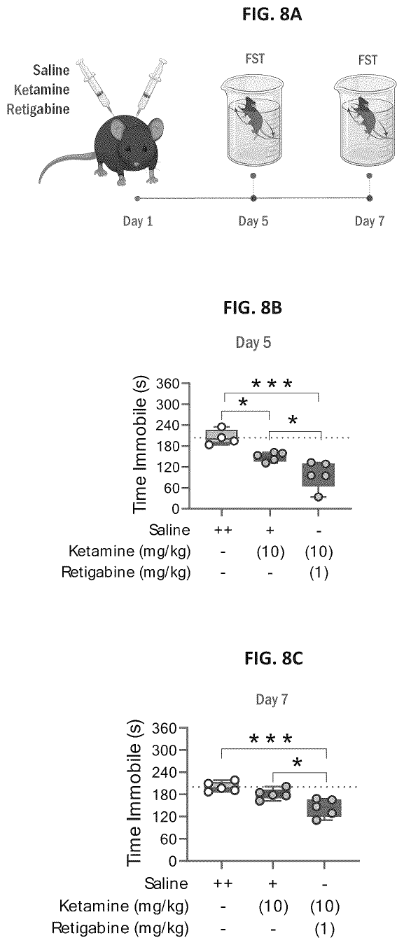

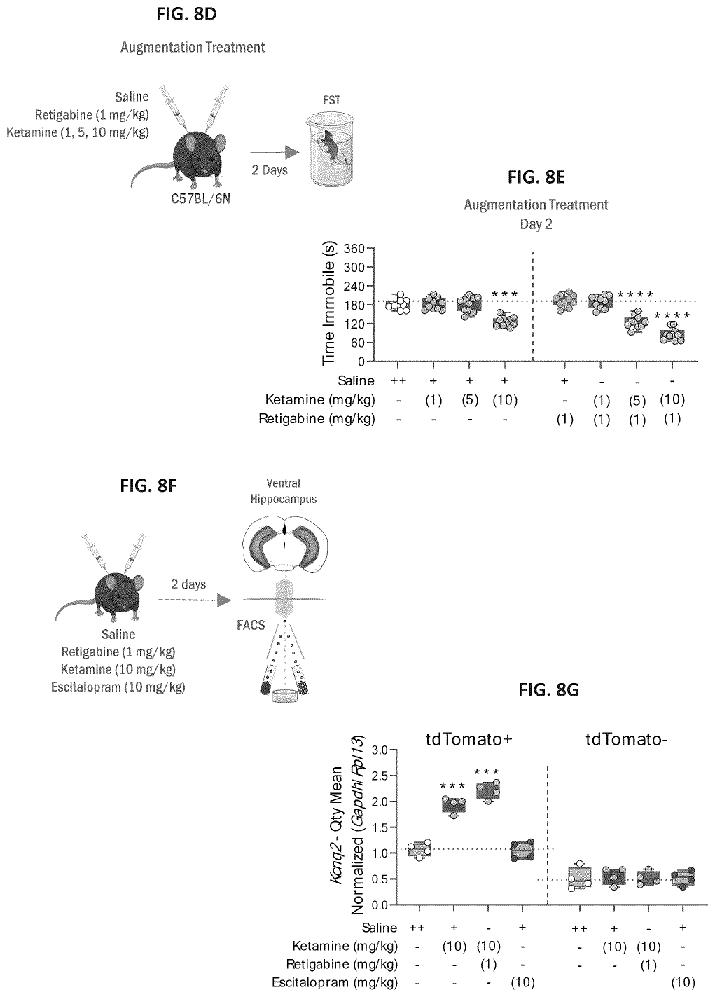

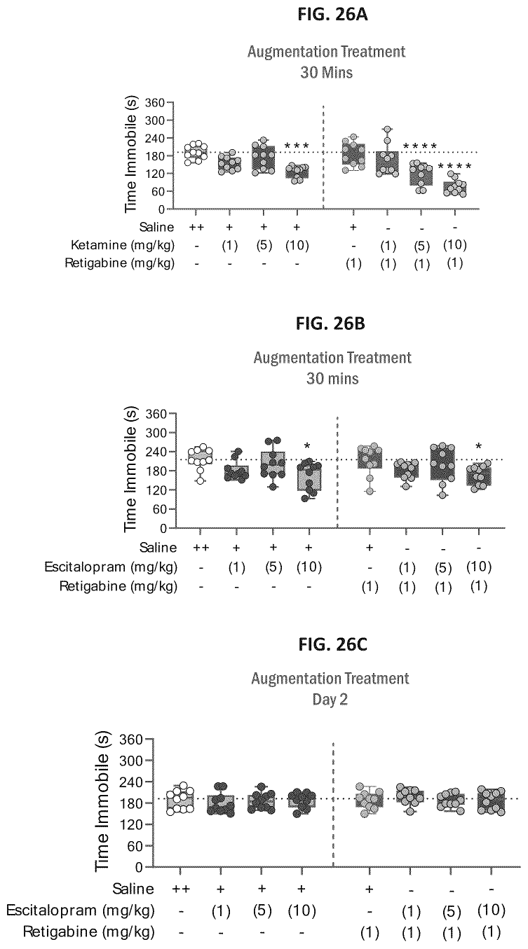

The present inventors have further demonstrated that systemic pharmacological manipulation of KCNQ channels modulate antidepressant-like behaviors in mice (see Example 7, herein below) and that the adjunctive treatment of ketamine and retigabine, a KCNQ activator, synergistically augments the antidepressant-like effects of ketamine (see Example 7, herein below). Specifically, the present inventors illustrated that while retigabine alone (at a dose of 1 mg/kg) had no improved effect on antidepressant-like behaviors in mice, i.e. exhibited an effect similar to saline, and ketamine is capable of reducing anxiety and stress while improving social behavior in mice, the combination of ketamine treatment with retigabine produces a synergistic improved antidepressant-like effects in treated mice (see Examples 8-9, herein below). This effect was evident at a dose of ketamine equivalent to the gold standard (i.e. 0.5 mg/kg by IV administration) as well as at a dose half of the gold standard (see FIG. 8E). Furthermore, the combined effect of ketamine and retigabine was sustained lasting longer than the effect of ketamine alone (see Example 9, herein below). The present inventors have further illustrated that this effect is specific to the combination of a NMDA receptor antagonist (e.g. ketamine) and a KCNQ channel activator (e.g. retigabine) and cannot be reproduced when retigabine is administered in combination with another antidepressant such as the selective serotonin reuptake inhibitor (SSRI) Escitalopram (see FIGS. 26A-C and 27A-B).

Taken together, these findings postulate the voltage-gated potassium channel KCNQ as a target for the treatment of mood disorders, such as major depressive disorder (MDD) and treatment-resistant patients. Furthermore, these findings provide a combination therapy of modulating KCNQ function (such as by using retigabine) in combination with an NMDA receptor blocker (such as ketamine) in the treatment of psychiatric disorders, such as in the depressive disorders MDD and treatment-resistant patients.

Thus, according to one aspect of the present invention there is provided a method of treating a psychiatric disorder in a subject in need thereof, the method comprising administering to the subject a therapeutically effective amount of an N-methyl-D-aspartate (NMDA) receptor antagonist and a therapeutically effective amount of a KCNQ channel activator, thereby treating the subject.

According to another aspect of the invention, there is provided a therapeutically effective amount of an NMDA receptor antagonist and a therapeutically effective amount of a KCNQ channel activator for use in treating a psychiatric disorder in a subject in need thereof.

The term “treating” refers to inhibiting or arresting the development of a disease, disorder or condition and/or causing the reduction, remission, or regression of a disease, disorder or condition or keeping a disease, disorder or medical condition from occurring (i.e. preventing) in a subject who may be at risk for the disease disorder or condition, but has not yet been diagnosed as having the disease disorder or condition. Those of skill in the art will understand that various methodologies and assays can be used to assess the development of a disease, disorder or condition, and similarly, various methodologies and assays may be used to assess the reduction, remission or regression of a disease, disorder or condition.

As used herein, the term “subject” or “subject in need thereof” includes mammals, such as human beings, male or female, at any age or gender who suffers from the pathology or is at risk to develop the pathology.

As used herein the phrase “psychiatric disorder” refers to a mental disorder or illness that interferes with the way a person behaves, interacts with others, and/or functions in daily life. Psychiatric disorders include mood disorders (e.g., depression of all forms and/or types, bipolar disorder, etc.), anxiety disorders, psychotic disorders (e.g., schizophrenia, personality disorders), as well as other mental disorders such as substance-related disorders, childhood disorders, dementia, multi-infarct dementia, autistic disorders, adjustment disorders, delirium, and Tourette's disorder as described in, e.g., the Diagnostic and Statistical Manual (DSM) of Mental Disorders, Fifth Edition (DSM-5), and further discussed below. Typically, such disorders have a complex genetic, biochemical, and/or environmental component.

According to one embodiment, the psychiatric disorder is a mood disorder.

According to one embodiment, a “mood disorder” refers to disruption of feeling, tone or emotional state experienced by an individual for an extensive period of time. Mood disorders include, but are not limited to, depression (i.e., depressive disorders), bipolar disorders, substance-induced mood disorders, alcohol-induced mood disorders, benzodiazepine-induced mood disorders, mood disorders due to general medical conditions, as well as many others. See, e.g., DSM-5 (www(dot)dsm5(dot)org), incorporated herein by reference. There are many general medical conditions that can trigger mood episodes, including, but not limited to, neurological disorders (e.g., dementias), metabolic disorders (e.g., electrolyte disturbances), gastrointestinal diseases (e.g., cirrhosis), endocrine disease (e.g., thyroid abnormalities), cardiovascular disease (e.g., heart attack), pulmonary disease (e.g., chronic obstructive pulmonary disease), cancer, autoimmune diseases (e.g., rheumatoid arthritis), and the like.

According to one embodiment, the psychiatric disorder is a depression-related disorder.

The term “depression” or “depressive disorder” or “depression-related disorder” includes a mood disorder involving any of the following symptoms: persistent sad, anxious, and/or “empty” mood; feelings of hopelessness and/or pessimism; feelings of guilt, worthlessness, and/or helplessness; loss of interest or pleasure in hobbies and activities that were once enjoyed; decreased energy, fatigue, and/or being “slowed down”; difficulty concentrating, remembering, and/or making decisions; insomnia, early-morning awakening, and/or oversleeping; loss of appetite and/or weight loss, overeating and/or weight gain; thoughts of death and/or suicide; suicide attempts; restlessness and/or irritability; persistent physical symptoms that do not respond to treatment, such as headaches, digestive disorders, and/or chronic pain; and combinations thereof. See, e.g., DSM-5 (described above).

Non-limiting examples of depression-related disorders include, but are not limited to, major depression disorder (MDD), atypical depression, melancholic depression, psychotic major depression or psychotic depression, catatonic depression, postpartum depression, seasonal affective disorder (SAD), chronic depression (dysthymia), severe depression, unipolar depression, double depression, depressive disorder not otherwise specified, depressive personality disorder (DPD), recurrent brief depression (RBD), minor depressive disorder (minor depression), premenstrual syndrome, premenstrual dysphoric disorder, depression caused by chronic medical conditions (e.g., cancer, chronic pain, chemotherapy, chronic stress), and combinations thereof. Various subtypes of depression are described in, e.g., DSM-5 (described above). In particular embodiments, the depression is major depression disorder (MDD). In certain instances, the methods of the present invention treat or alleviate one or more symptoms of depression. In certain other instances, the methods of the present invention treat depression.

According to a specific embodiment, the depression-related disorder comprises a major depression disorder (MDD). According to one embodiment, the MDD is associated with suicidal ideation.

According to a specific embodiment, the depression-related disorder comprises a treatment-resistant depression (TRD). TRD typically refers to inadequate response to at least one antidepressant therapy of adequate doses and duration. Such an adequate dose and duration is well known to one of skill in the art.

According to one embodiment, the psychiatric disorder is a bipolar disorder.

According to one embodiment, “bipolar disorder” refers to a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM-5 (described above). Bipolar disorder is also known as manic depression.

Non-limiting examples of bipolar disorders include, but are not limited to, mania, acute mania, severe mania, hypomania, depression, moderate depression, dysthymia, severe depression, episodes of mania and/or depression, psychosis/psychotic symptoms (e.g. hallucinations, delusions), mixed bipolar state, bipolar I disorder (mania with or without major depression), bipolar II disorder (hypomania with major depression), rapid-cycling bipolar disorder, Cyclothymia and/or Bipolar Disorder Not Otherwise Specified (BD-NOS). See, e.g., DSM-5 (described above).

According to one embodiment, the psychiatric disorder is a Schizophrenia disorder.

According to one embodiment, “schizophrenia” refers to a psychiatric disorder involving a withdrawal from reality by an individual. Symptoms comprise for at least a part of a month two or more of the following symptoms: delusions, hallucinations, disorganized speech, grossly disorganized or catatonic behavior, or negative symptoms (i.e., affective flattening, alogia, or avolition). Schizophrenia encompasses disorders such as, e.g., schizoaffective disorders. Diagnosis of schizophrenia is described in, e.g., DSM-5 (described above). Types of schizophrenia include, but are not limited to, paranoid, disorganized, catatonic, undifferentiated, and residual. See, e.g., DSM-5 (described above).

According to one embodiment, the psychiatric disorder is a psychotic disorder.

According to one embodiment, a “psychotic disorder” refers to a condition that affects the mind, resulting in at least some loss of contact with reality. Symptoms of a psychotic disorder include, e.g., hallucinations, changed behavior that is not based on reality, delusions, and the like. See, e.g., DSM-5 (described above). Schizophrenia, schizoaffective disorder, schizophreniform disorder, delusional disorder, brief psychotic disorder, substance-induced psychotic disorder, and shared psychotic disorder are non-limiting examples of psychotic disorders.

According to one embodiment, the psychiatric disorder is an anxiety disorder.

According to one embodiment, an “anxiety disorder” or “anxiety” refers to a condition characterized by feelings of worry, nervousness, unease, and/or tension, typically about an imminent event or something with an uncertain outcome. Symptoms of anxiety include, without being limited to, fear, panic, heart palpitations, shortness of breath, fatigue, nausea, headaches (e.g., tension headaches), tachycardia, muscle weakness and/or tension, chest pain, stomach aches, pallor, sweating, trembling, pupillary dilation, panic attacks, and combinations thereof. See, e.g., DSM-5 (described above). An anxiety disorder may be characterized by chronic anxiety, such as a Generalized Anxiety Disorder (GAD), or may be an acute anxiety, such as a panic attack or panic syndrome.

According to one embodiment, the psychiatric disorder is an autism spectrum disorder.

According to one embodiment, autism spectrum disorder refers to a spectrum of neurodevelopmental disorders characterized by impaired social interaction and communication accompanied by repetitive and stereotyped behavior. Autism includes a spectrum of impaired social interaction and communication, however, the disorder can be roughly categorized into “high functioning autism” or “low functioning autism”, depending on the extent of social interaction and communication impairment. Individuals diagnosed with “high functioning autism” have minimal but identifiable social interaction and communication impairments (e.g., Asperger's syndrome). Additional information on autism spectrum disorders can be found in, e.g., DSM-5 (described above); Sicile-Kira and Grandin, Autism Spectrum Disorders: The Complete Guide to Understanding Autism, Asperger's Syndrome, Pervasive Developmental Disorder, and Other ASDs, 2004, Perigee Trade; and Duncan et al., Autism Spectrum Disorders [Two Volumes]: A Handbook for Parents and Professionals, 2007, Praeger.

According to one embodiment, the psychiatric disorder is a neuroimmune-based psychiatric disorder. Non-limiting examples of neuroimmune-based psychiatric disorders include, but are not limited to, mood disorders such as depression (e.g., major depressive disorder) and bipolar disorder, schizophrenia, autism spectrum disorder, Pediatric Acute-onset Neuropsychiatric Syndrome (PANS) and Pediatric autoimmune neuropsychiatric disorder (PANDAS).

Additionally or alternatively, the methods of the present invention may be used towards the treatment of any one of, but not limited to, phobic syndromes of all types, e.g. social phobia including e.g. social anxiety disorder; anxiety disorders including agoraphobia, panic attack; stress disorders, e.g. post-traumatic stress disorder (PTSD); neurotic disorders (e.g., obsessive-compulsive disorder (OCD) and anxiety); somatoform disorders; personality disorders including e.g. dissocial personality disorder, paranoid personality disorders, schizoid personality disorders, schizotypal personality disorders, antisocial personality disorders, borderline personality disorders, histrionic personality disorders, narcissistic personality disorders; compulsive behavior; psychosis; intermittent explosive disorder (IED); Pyromania; Kleptomania; impulse control and addiction or substance-related disorders e.g. drug dependence [e.g., alcohol, psychostimulants (e.g., crack, cocaine, speed, and meth), opioids, and nicotine]; fetal alcohol syndrome; attention deficit hyperactivity disorder (ADHD); stress; fatigue; epilepsy; pain including e.g. headache, acute pain, chronic pain; neuropathies; cereborischemia; dementia (e.g. Alzheimer's type and multi-infarct dementia); Parkinson's Disease; memory loss (e.g. Alzheimer's Disease); cognition impairment (e.g. impaired cognitive function); sleep disorders including e.g. insomnia, early-morning awakening, and/or oversleeping; eating disorders including e.g. bulimia, anorexia, body image distortion, binge-eating disorders; Tourette's syndrome; Blepharospasm; Tic disorder; Impulse control disorder (ICD); childhood disorders such as e.g. pervasive developmental disorder (PDD); or any disease or condition associated therewith, e.g. multiple sclerosis, movement disorders, growth disorders, reproduction disorders, adjustment disorders, delirium (e.g. such as those associated with depression and anxiety). Additional disorders are described in, e.g., the Diagnostic and Statistical Manual (DSM) of Mental Disorders, Fifth Edition (DSM-5) (described above).

As mentioned, the methods of the invention are affected by administering to the subject an N-methyl-D-aspartate (NMDA) receptor antagonist.

The term “N-methyl-D-aspartate” or “NMDA” receptor refers to postsynaptic, ionotropic receptor found in neurons that is responsive to, inter alia, the excitatory amino acids glutamate and glycine (or D-serine). Typically, in response to the neurotransmitter glutamate and concurrent binding of glycine (or D-serine), NMDA receptors (found at most excitatory synapses) are activated and allow a voltage-dependent flow of sodium (Na⁺) and small amounts of calcium (Ca²⁺) ions into the cell and potassium (K+) out of the cell.

NMDA receptors are typically comprised of heterotetramers of subunits encoded by three gene families: NR1, NR2 and NR3. The NR1 family consists of one gene with eight isomers, and is an essential structural component found in all tetramers (GluN1). The NR2 family consists of four genes encoding four GluN2 subunits (GluN2A-D), which contribute to four diheteromeric NMDAR subtypes that have divergent physiological and pathological roles. The NR3 proteins (GluN3) consist of two members (A and B) and function as negative components when included in receptor structures. The composition of different subunits and splicing variants form the primary basis of the functional diversity of NMDA receptors as discussed in Bai and Hoffman, “Transcriptional Regulation of NMDA Receptor Expression” In: Van Dongen A M, editor. Biology of the NMDA Receptor. Boca Raton (Fla.): CRC Press/Taylor & Francis; Chapter 5 (2009). Typically, GluN2 comprises the binding site for glutamate, while GluN1 (or GluN3) contains the Glycine/D-serine binding site.

The term “NMDA receptor antagonist” refers to a compound that reduces the flow of cations (Na⁺, K⁺, Ca²⁺) through the NMDA receptor. The NMDA receptor antagonists comprise four categories of compounds: competitive antagonists, which bind to and block the binding site of the neurotransmitter glutamate; glycine antagonists, which bind to and block the glycine site; noncompetitive antagonists, which inhibit NMDA receptors by binding to allosteric sites; and uncompetitive antagonists or channel blockers, which block the ion channel by binding to a site within it.

Any NMDA receptor antagonist is contemplated for use in the compositions and methods of the present invention.

In one embodiment of the invention, the NMDA receptor antagonist is a noncompetitive antagonist of the NMDA receptor. “Noncompetitive antagonists” refer to compounds which require the binding of glycine and glutamate then the compound can bind to an allosteric site of the channel and block the flow of cations. Examples of noncompetitive antagonists include but are not limited to: ketamine, amantadine, tiletamine, phencyclidine (PCP), PCP hydrochloride functional derivatives, dizocilpine (MK-801), Argiotoxin-636, dextrorphan, Dexanabinol (HU-211), Rhynchophylline, and/or analogs and/or derivative thereof (e.g. functional derivatives or analogs).

In one embodiment of the invention, the NMDA receptor antagonist is an uncompetitive antagonist (channel blocker) of the NMDA receptor. “Uncompetitive antagonists” refer to compounds which require the binding of an agonist of the NMDA receptor (e.g. glycine, glutamate) and the channel opening to access their blocking site. The uncompetitive channel blocker then becomes trapped within the NMDA receptor. Examples of uncompetitive antagonists of the NMDA receptor include but are not limited to: memantine, aptiganel (CNS 1102, Cerestat), Ifenprodil, Lanicemine (AZD6765), Remacemide, DQP-1105, and/or analogs and/or derivatives thereof (e.g. functional derivatives or analogs).

Additional non-limiting examples of NMDA receptor antagonists include, but are not limited to, dextromethorphan (AVP-786), Traxoprodil (CP-101606), Rislenemdaz (MK-0657), nitrous oxide, 4-chlorokynurenine (4-Cl-KYNA) (AV-101), D-cycloserine (DCS), rapastinel (GLYX-13; BV-102), dynorphin A(1-13), eliprodil, felbamate, fluorofelbamate, Conantokin-G, —R, NVP-AAM077, R025-6981, Selfotel (CGS-19755), TCN-201, and/or analogs and/or derivatives thereof (e.g. functional derivatives or analogs).

NMDA receptor antagonist have been further described in the art, for example in Jelen et al., Ther Adv Psychopharmacol (2018) 8(3): 95-98), incorporated herein by reference.

According to one embodiment, the NMDA receptor antagonist is ketamine and/or analogs and/or derivatives thereof (or any analog or derivative of any of the compounds contemplated herein).

The term “analog” as used herein broadly refers to the modification or substitution of one or more chemical moieties on a parent compound and may include functional derivatives, positional isomers, tautomers, zwitterions, enantiomers, diastereomers, racemates, isosteres or stereochemical mixtures thereof.

The term “derivative” as used herein refers to a compound which possesses similar IC50 values and kinetics properties as the parent compound (e.g. ketamine or retigabine, discussed below) to its receptor (e.g. NMDA receptor or KCNQ channel, discussed below).

According to a specific embodiment, the ketamine comprises an active metabolite of ketamine. Exemplary active metabolites of ketamine include, but are not limited to, Norketamine and Hydroxynoketamine, as discussed in Hashimoto, Psychiatry and Clinical Neurosciences (2019) 73: 613-627, incorporated herein by reference.

According to a specific embodiment, the ketamine comprises an enantiomer of ketamine or of said active metabolite thereof. Exemplary enantiomers include, but are not limited to, (R,S)-ketamine, (R)-ketamine, (S)-ketamine, (R)-Norketamine, (S)-Norketamine, (2R,6R)-hydroxynorketamine (HNKet) and (2S,6S)-hydroxynorketamine, as discussed in Hashimoto, Psychiatry and Clinical Neurosciences (2019) 73: 613-627, incorporated herein by reference.

According to a specific embodiment, the ketamine is Racemic ketamine, i.e. a mixture of (R)-ketamine and (S)-ketamine, also referred to herein as (R,S)-ketamine.

According to a specific embodiment, the ketamine is Esketamine (Spravato), i.e. an (S)-enantiomer of ketamine.

According to a specific embodiment, the ketamine is ketamine hydrochloride.

As mentioned, the methods of the invention are affected by administering to the subject a KCNQ channel activator.

The term “KCNQ channel” refers to the voltage-gated potassium channels encoded by the KCNQ genes, also designated Kv7 potassium channels. KCQN genes encode family members of the Kv7 potassium channel family (also referred to herein as subunits) including Kv7.1 (KCNQ1), Kv7.2 (KCNQ2), Kv7.3 (KCNQ3), Kv7.4 (KCNQ4), and Kv7.5 (KCNQ5). All KCNQ channels share a typical topological design, consisting of a functional channel formed by four subunits, each comprising six transmembrane domains termed S1 to S6, with the voltage-sensing domain (VSD) being located within the first four segments (S1-S4), and the last two segments (S5-S6) and linker comprise the pore-forming domain (PD), a short N terminus and a long C terminus, both intracellular. Furthermore, the KCNQ channels may be homomeric or heteromeric comprising one or more types of the subunits Kv7.1-Kv7.5.

In the brain, the expression of Kv7.2, Kv7.3 and Kv7.5 subunits are most abundant. The Kv7.4 subunit has the most restricted regional expression in the brain and is only present in discrete nuclei of the brainstem. KCNQ channels comprising subunits Kv7.2 to Kv7.5 typically produce the so called ‘M-current’, a low-threshold gating, slowly activating current that has profound effects on synaptic plasticity and neuronal excitability and acts as a brake for repetitive firing.

Kv7.2 subunits are capable of forming homomeric KCNQ channels formed solely by Kv7.2 subunits, but heteromerization with Kv7.3 subunits increases the M-currents, mostly due to a more efficient surface targeting and expression of functional channels. Kv7.3 subunits typically form heteromers with Kv7.2 or Kv7.5, while Kv7.4 is less able to heteromerize with Kv7.3 or Kv7.2 but assembles readily with Kv7.5. It has been shown that these heteromers produce larger currents than homomeric Kv7.4 channels.

According to a specific embodiment, the KCNQ channel comprises Kv7.2 protein which is encoded by Kcnq2 gene.

The term “KCNQ channel activator” or “KCNQ activating compound” as used herein refers to a compound capable of activating one or more voltage gated KCNQ potassium channels comprising subunits of the Kv7 family. A KCNQ activator is typically capable of binding to a KCNQ channel and triggering one or more effects, such as stabilizing the open conformation of the channel and facilitating series of conformational changes to open the channel, increased channel open times, and decreased longest closed times. As a result of these effects, the transportation of ions through the channel is increased and the M-current is increased.

In one embodiment of the invention, the KCNQ activator is capable of activating one or more of the homomeric KCNQ channels comprising one type of subunit selected from the group of Kv7.2, Kv7.3, Kv7.4 and Kv7.5.

In one embodiment of the invention, the KCNQ channel activator is capable of activating one or more of the heteromeric KCNQ channels selected from the group of KCNQ channels comprising Kv7.2 and Kv7.3 subunits (Kv7.2/3 channels), comprising Kv7.3 and Kv7.4 subunits (Kv7.3/4 channels), comprising Kv7.3 and Kv7.5 subunits (Kv7.3/5 channels) or comprising Kv7.4 and Kv7.5 subunits (Kv7.4/5 channels).

According to a specific embodiment, the KCNQ activator activates a KCNQ channel comprising a Kv7.2 subunit. The KCNQ may be homomeric for the Kv7.2 subunit (i.e. Kv7.2/Kv7.2). Alternatively, the KCNQ may be heteromeric for the Kv7.2 subunit selected from the group of KCNQ channels comprising Kv7.2 and Kv7.3 subunits (Kv7.2/3 channels), comprising Kv7.2 and Kv7.4 subunits (Kv7.2/4 channels), or comprising Kv7.2 and Kv7.5 subunits (Kv7.2/5 channels).

Any KCNQ activating compound is contemplated for use in the compositions and methods of the present invention.

Non-limiting examples of a KCNQ channel activators include, but are not limited to, retigabine (ezogabine), flupirtine, ICA-27243, the racemic mixture BMS-204352 (Maxipost), Acrylamide (S)-1, Acrylamide (S)-2, diclofenac, meclofenamic acid, derivatives of diclofenac or meclofenamic acid (e.g., NH6, NH29), ICA-105665, ML213, NS15370, AaTXKP(2-64), ICA-27243, ICA-069673, ICA-105665, N-ethylmaleimide, zinc pyrithione, hydrogen peroxide and/or analogs and/or derivatives thereof (e.g. functional derivatives or analogs, such as the S enantiomer of BMS-204352).

KCNQ activating compounds have been described in the art (for example in Barrese V. et al. Annu. Rev. Pharmacol. Toxicol. (2018) 58: 625-648, Wulff al. Nat Rev Drug Discov. (2009) 8(12):982-1001 and in Xiong et al. Trends Pharmacol Sci. (2008) 29(2):99-107, all of which are incorporated herein in their entirety).

According to one embodiment, the KCNQ channel activator is retigabine (ezogabine) and/or analogs and/or derivatives thereof (e.g. functional derivatives or analogs).

According to a specific embodiment, the NMDA receptor antagonist (e.g. ketamine) and the KCNQ channel activator (e.g. retigabine (ezogabine)) may be linked by at least one chemical bond. Linking the NMDA receptor antagonist (e.g. ketamine) and the KCNQ channel activator (e.g. retigabine (ezogabine)) can increase the therapeutic efficacy of these molecules.

Any method known in the art for linking molecules can be used in accordance with the present invention. Such methods typically involve forming covalent bonds between chemically-compatible reactive groups that are intrinsic or generated within each molecule, using methodologies well known in the art. An exemplary method involves using click chemistry (described e.g. in Schreiber and Smith, Nature Reviews Chemistry (2019) 3: 393-400, incorporated herein by reference).

According to some of any of the embodiments described herein, any of the compounds described herein, e.g., an NMDA receptor antagonist e.g. ketamine and/or analogs and/or derivatives thereof, a KCNQ channel activator, e.g. retigabine (ezogabine) and/or analogs and/or derivatives thereof, can be in a form of a pharmaceutically acceptable salt thereof.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, and/or to improve its stability, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., an amine-containing group) group of the compound which is in a positively charged form (e.g., wherein the basic group is protonated), in combination with at least one counter-ion, derived from the selected base, that forms a pharmaceutically acceptable salt; and/or at least one acidic group of the compound which is in a negatively charged form (e.g., de-protonated) in combination with at least one counter-ion, derived from the selected base, that forms a pharmaceutically acceptable salt. Oxonium positively charged ions and a counter anion are also contemplated.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein, e.g., an NMDA receptor antagonist, ketamine and/or analogs and/or derivatives thereof, a KCNQ channel activator, retigabine (ezogabine) and/or analogs and/or derivatives thereof.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

According to one embodiment, NMDA receptor antagonist (e.g. ketamine) and/or KCNQ channel activator (e.g. retigabine (ezogabine)) is administered in a single dose.

According to one embodiment, NMDA receptor antagonist (e.g. ketamine) and/or KCNQ channel activator (e.g. retigabine (ezogabine)) is administered in 2, 3, 4, 5, 6, 7, 8, 9, 10 or more doses.

According to one embodiment, NMDA receptor antagonist (e.g. ketamine) and/or KCNQ channel activator (e.g. retigabine (ezogabine)) is administered once a month, twice a month, once a week, twice a week, three times a week, four times a week, five times a week, once a day, twice a day, three times a day, four times a day, or five times a day.

According to one embodiment, NMDA receptor antagonist (e.g. ketamine) and/or KCNQ channel activator (e.g. retigabine (ezogabine)) is administered at least once a day (e.g. 1-5 times a day, e.g. 1-3 times a day) on consecutive days for at least a week, for at least 10 days, for at least 14 days, for at least a month, for at least 3 months, for at least 6 months, for at least a year, or more as needed.

According to one embodiment, NMDA receptor antagonist (e.g. ketamine) and/or KCNQ channel activator (e.g. retigabine (ezogabine)) is administered at least once a week (e.g. 2, 3, 4, 5 times a week) on consecutive weeks for at least two weeks, for at least one month, for at least 3 months, for at least 6 months, for at least a year, or more as needed.

According to one embodiment, NMDA receptor antagonist (e.g. ketamine) and/or KCNQ channel activator (e.g. retigabine (ezogabine)) is administered once a week.

According to one embodiment, NMDA receptor antagonist (e.g. ketamine) and/or KCNQ channel activator (e.g. retigabine (ezogabine)) is administered twice a week.

According to one embodiment, NMDA receptor antagonist (e.g. ketamine) and/or KCNQ channel activator (e.g. retigabine (ezogabine)) is administered 1-2 times a week.

According to one embodiment, NMDA receptor antagonist (e.g. ketamine) and/or KCNQ channel activator (e.g. retigabine (ezogabine)) is administered 2-3 times a week.

According to one embodiment, NMDA receptor antagonist (e.g. ketamine) and/or KCNQ channel activator (e.g. retigabine (ezogabine)) is administered 4-5 times a week.

According to one embodiment, NMDA receptor antagonist (e.g. ketamine) and/or KCNQ channel activator (e.g. retigabine (ezogabine)) is administered 5-6 times a week.

According to one embodiment, NMDA receptor antagonist (e.g. ketamine) and/or KCNQ channel activator (e.g. retigabine (ezogabine)) is administered daily.

According to one embodiment, NMDA receptor antagonist (e.g. ketamine) and/or KCNQ channel activator (e.g. retigabine (ezogabine)) is administered for one week.

According to one embodiment, NMDA receptor antagonist (e.g. ketamine) and/or KCNQ channel activator (e.g. retigabine (ezogabine)) is administered for 2 or more weeks (e.g. on consecutive weeks), 2 weeks, 3 weeks, 4 weeks, 5 weeks, 6 weeks or more.

According to one embodiment, NMDA receptor antagonist (e.g. ketamine) and/or KCNQ channel activator (e.g. retigabine (ezogabine)) is administered once a month on consecutive months for at least two months, for at least 3 months, for at least 4 months, for at least 5 months, for at least 6 months, for at least a year, or more as needed.

According to one embodiment, NMDA receptor antagonist (e.g. ketamine) and/or KCNQ channel activator (e.g. retigabine (ezogabine)) is administered for a chronic treatment.

According to one embodiment, NMDA receptor antagonist (e.g. ketamine) and/or KCNQ channel activator (e.g. retigabine (ezogabine)) is administered for an acute treatment.

According to one embodiment, NMDA receptor antagonist (e.g. ketamine) and/or KCNQ channel activator (e.g. retigabine (ezogabine)) is administered for up to 1-40 weeks, e.g. for up to 1-30 weeks, for up to 1-26 weeks, e.g. for up to 1-21 weeks, e.g. for up to 1-17 weeks, e.g. for up to 1-14 weeks, e.g. for up to 1-10 weeks, e.g. for up to 1-8 weeks, e.g. for up to 1-6 weeks, e.g. for up to 2-4 weeks.

According to one embodiment, NMDA receptor antagonist (e.g. ketamine) and/or KCNQ channel activator (e.g. retigabine (ezogabine)) is administered for up to 1, 2, 3, 4, 5, 6, 7, 8, 9, 10, 12, 14, 17, 21, 26, 30, 35 or 40 weeks.

According to one embodiment, NMDA receptor antagonist (e.g. ketamine) and/or KCNQ channel activator (e.g. retigabine (ezogabine)) is administered for up to 1-60 administrations, e.g. for up to 1-2 administrations, e.g. for up to 1-5 administrations, e.g. for up to 1-10 administrations, e.g. for up to 5-10 administrations, e.g. for up to 5-15 administrations, e.g. for up to 10-15 administrations, e.g. for up to 15-20 administrations, e.g. for up to 20-30 administrations, e.g. for up to 30-40 administrations, e.g. for up to 40-50 administrations or e.g. for up to 50-60 administrations.

According to one embodiment, NMDA receptor antagonist (e.g. ketamine) and/or KCNQ channel activator (e.g. retigabine (ezogabine)) is administered for up to 1, 2, 3, 4, 5, 6, 7, 8, 9, 10, 12, 14, 17, 21, 26, 30, 40, 50 or 60 administrations.

According to one embodiment, NMDA receptor antagonist (e.g. ketamine) and/or KCNQ channel activator (e.g. retigabine (ezogabine)) are administered concomitantly.

According to one embodiment, NMDA receptor antagonist (e.g. ketamine) and/or KCNQ channel activator (e.g. retigabine (ezogabine)) are administered subsequently to one another (e.g. minutes, hours or days apart, such as but not limited to, between 5 and 15 minutes, between 15 and 30 minutes, between 30 minutes and 60 minutes, between 60 and 90 minutes, between 90 and 120 minutes, between 120 and 180 minutes, between 180 and 240 minutes, between 4-8 hours, between 8-12 hours, between 12-24 hours, between 24-48 hours, or between 48-96 hours).

It will appreciated that the number of administrations for each of NMDA receptor antagonist (e.g. ketamine) and the KCNQ channel activator (e.g. retigabine (ezogabine)) may differ, the total length of time for treatment by each may differ, and the route of administration may differ, as long as the effect of the KCNQ channel activator (e.g. retigabine (ezogabine)) augments the effects of the NMDA receptor antagonist (e.g. ketamine) and/or lowers the effective dose of the NMDA receptor antagonist (e.g. ketamine) needed to achieve a therapeutic efficacy, as further discussed herein below.

According to one embodiment, a therapeutically effective amount of an NMDA receptor antagonist (e.g. ketamine) and of a KCNQ channel activator (e.g. retigabine (ezogabine)) is calculated in order to reach a desired effect such as anti-depressive effect, decrease in anxiety, decrease in stress, decrease in hallucinations and/or decrease in aggressiveness. Methods for assessing the efficacy of the treatment are readily measurable by routing procedures familiar to a physician. Furthermore, a therapeutically effective amount is calculated based on the route of administration. Examples of adapted routes of administration include, but are not limited to: intramuscular (IM), subcutaneous (SC), intravenous (IV), parenteral, intranasal and oral administration, as further discussed herein below. It will be further appreciated that dosing can be adjusted, e.g. upscaled, downscaled or tapered, as needed to reach the therapeutic effect, i.e. treatment of a psychiatric disease or condition. Such a determination is well within the knowledge of one of skill in the art.

According to one embodiment, the administration dose of an NMDA receptor antagonist (e.g. ketamine) and/or a KCNQ channel activator (e.g. retigabine (ezogabine)) is determined by the skilled artisan and personally adapted to each subject.

As mentioned, the present inventors have uncovered that treating a subject with the combination of ketamine and retigabine (ezogabine) provides a strong anti-depressant effect and reduces the levels of ketamine needed to achieve an effective psychiatric therapy.

Thus, according to one embodiment, the methods of the invention are affected by administering to the subject ketamine (i.e. an NMDA receptor antagonist) and retigabine (ezogabine) (i.e. a KCNQ channel activator).

According to one embodiment, ketamine is administered in a sub-anesthetic dose, i.e. a dosage of ketamine not causing any loss of consciousness.

According to one embodiment, the sub-anesthetic dose of ketamine is lower or equal to 15 mg/kg body weight, 14 mg/kg body weight, 13 mg/kg body weight, 12 mg/kg body weight, 11 mg/kg body weight, 10 mg/kg body weight, 9 mg/kg body weight, 8 mg/kg body weight, 7 mg/kg body weight, 6 mg/kg body weight, 5 mg/kg body weight, 4 mg/kg body weight, 3 mg/kg body weight, 2 mg/kg body weight, 1 mg/kg body weight, or 0.5 mg/kg body weight, wherein day is calculated per 24 hours.

According to one embodiment, the sub-anesthetic dose of ketamine is calculated per daily dose. For example, the sub-anesthetic dose of ketamine may be lower or equal to 15 mg/kg body weight/day, 14 mg/kg body weight/day, 13 mg/kg body weight/day, 12 mg/kg body weight/day, 11 mg/kg body weight/day, 10 mg/kg body weight/day, 9 mg/kg body weight/day, 8 mg/kg body weight/day, 7 mg/kg body weight/day, 6 mg/kg body weight/day, 5 mg/kg body weight/day, 4 mg/kg body weight/day, 3 mg/kg body weight/day, 2 mg/kg body weight/day, or 1 mg/kg body weight/day, or 0.5 mg/kg body weight/day, wherein day is calculated per 24 hours.

According to one embodiment, a therapeutically effective amount of ketamine is an amount capable of alleviation of symptoms of psychiatric disorders, such as depression-related disorders.

According to one embodiment, a therapeutically effective amount of ketamine comprises a dose lower than the Gold standard administered to psychiatric patients (e.g. a dose of 0.5-1.0 mg/kg body weight per 40-60 minutes via intravenous (IV) administration, e.g. infused twice weekly). According to one embodiment, a therapeutically effective amount of ketamine comprises a dose lower than the Gold standard administered to psychiatric patients (e.g. a dose of 0.5 mg/kg body weight per 40 minutes via intravenous (IV) administration, e.g. infused twice weekly).

According to one embodiment, the therapeutically effective amount of ketamine is lower or equal to 5 mg/kg body weight, 4 mg/kg body weight, 3 mg/kg body weight, 2 mg/kg body weight, 1.5 mg/kg body weight, 1 mg/kg body weight, 0.75 mg/kg body weight, 0.5 mg/kg body weight, 0.4 mg/kg body weight, 0.3 mg/kg body weight, 0.2 mg/kg body weight or 0.1 mg/kg body weight, per 2-100 minutes via intravenous (IV) administration (e.g. per 5-60 minutes, per 5-50 minutes, e.g. per 15-45 minutes, e.g. per 30-45 minutes, e.g. per 40-60 minutes via IV administration).

According to one embodiment, the therapeutically effective amount of ketamine is lower or equal to 1.0 mg/kg body weight, 0.75 mg/kg body weight, 0.5 mg/kg body weight, 0.4 mg/kg body weight, 0.3 mg/kg body weight, 0.2 mg/kg body weight or 0.1 mg/kg body weight, per 40-60 minutes via intravenous (IV) administration.

According to a specific embodiment, the therapeutically effective amount of ketamine is lower or equal to 0.5 mg/kg body weight per 40-60 minutes via intravenous (IV) administration.

According to one embodiment, a therapeutically effective amount of ketamine comprises a dose at least 10%, at least 20%, at least 30%, at least 40%, at least 50%, at least 60%, at least 70%, at least 80%, or at least 90% lower than that of the Gold standard administered to psychiatric patients (e.g. a dose of 0.5-1.0 mg/kg body weight per 40-60 minutes via intravenous (IV) administration, e.g. infused twice weekly).

According to one embodiment, a therapeutically effective amount of ketamine comprises a dose at least 50% lower than that of the Gold standard administered to psychiatric patients (e.g. a dose of 0.5-1.0 mg/kg body weight per 40-60 minutes via intravenous (IV) administration, e.g. infused twice weekly).

According to one embodiment, a therapeutically effective amount of ketamine comprises a dose of no more than 0.4 mg/kg body weight, 0.3 mg/kg body weight, 0.2 mg/kg body weight, 0.1 mg/kg body weight for IV administration (e.g. per 40-60 minutes IV administration).

According to one embodiment, a therapeutically effective amount of ketamine comprises a dose of no more than 0.25 mg/kg body weight for IV administration (e.g. per 40-60 minutes of IV administration).

According to one embodiment, a therapeutically effective amount of ketamine comprises a dose of 0.01-15 mg/kg body weight (e.g. 0.01-0.05 mg/kg body weight, 0.01-0.1 mg/kg body weight, 0.1-0.3 mg/kg body weight, 0.3-0.5 mg/kg body weight, 0.5-0.7 mg/kg body weight, 0.7-0.9 mg/kg body weight, 1.0-1.5 mg/kg body weight, 1.5-2.0 mg/kg body weight, 2.0-2.5 mg/kg body weight, 2.5-3.0 mg/kg body weight, 3.0-3.5 mg/kg body weight, 3.5-4.0 mg/kg body weight, 4.0-4.5 mg/kg body weight, 4.5-5.0 mg/kg body weight, 5.0-6.0 mg/kg body weight, 6.0-7.0 mg/kg body weight, 7.0-8.0 mg/kg body weight, 8.0-9.0 mg/kg body weight, 9.0-10.0 mg/kg body weight, 10.0-12.0 mg/kg body weight, or 12.0-15.0 mg/kg body weight).

According to one embodiment, a therapeutically effective amount of ketamine comprises a dose of no more than 0.75 mg/kg body weight, 0.5 mg/kg body weight, e.g. 0.4 mg/kg body weight, 0.3 mg/kg body weight, 0.2 mg/kg body weight or 0.1 mg/kg body weight.

According to one embodiment, the therapeutically effective amount of ketamine is calculated per daily dose. For example, the therapeutically effective amount of ketamine may comprise a dose of 0.01-15 mg/kg body weight/day (e.g. 0.01-0.05 mg/kg body weight/day, 0.01-0.1 mg/kg body weight/day, 0.1-0.3 mg/kg body weight/day, 0.3-0.5 mg/kg body weight/day, 0.5-0.7 mg/kg body weight/day, 0.7-0.9 mg/kg body weight/day, 1.0-1.5 mg/kg body weight/day, 1.5-2.0 mg/kg body weight/day, 2.0-2.5 mg/kg body weight/day, 2.5-3.0 mg/kg body weight/day, 3.0-3.5 mg/kg body weight/day, 3.5-4.0 mg/kg body weight/day, 4.0-4.5 mg/kg body weight/day, 4.5-5.0 mg/kg body weight/day, 5.0-6.0 mg/kg body weight/day, 6.0-7.0 mg/kg body weight/day, 7.0-8.0 mg/kg body weight/day, 8.0-9.0 mg/kg body weight/day, 9.0-10.0 mg/kg body weight/day, 10.0-12.0 mg/kg body weight/day, or 12.0-15.0 mg/kg body weight/day).

According to one embodiment, a therapeutically effective amount of ketamine comprises a dose of 0.1-1.0 mg/kg body weight.

According to one embodiment, a therapeutically effective amount of ketamine comprises a dose of 0.1-0.75 mg/kg body weight.

According to one embodiment, a therapeutically effective amount of ketamine comprises a dose of 0.1-0.5 mg/kg body weight.

According to one embodiment, a therapeutically effective amount of ketamine comprises a dose of 0.1 mg/kg body weight, 0.2 mg/kg body weight, 0.3 mg/kg body weight, 0.4 mg/kg body weight, 0.5 mg/kg body weight, 0.6 mg/kg body weight, 0.7 mg/kg body weight, 0.8 mg/kg body weight, 0.9 mg/kg body weight, 1.0 mg/kg body weight, 1.25 mg/kg body weight, 1.5 mg/kg body weight, 1.75 mg/kg body weight, 2.0 mg/kg body weight, or 2.5 mg/kg body weight.

According to a specific embodiment, the therapeutically effective amount of ketamine (e.g. ketamine hydrochloride) comprises a dose of 0.1-1.0 mg/kg body weight (e.g. 0.1-0.75 mg/kg body weight, e.g. 0.1-0.4 mg/kg body weight) for intravenous administration.

According to a specific embodiment, ketamine (e.g. ketamine hydrochloride) is administered via intravenous (IV) administration at a dose of 0.1-1.0 mg/kg body weight (e.g. 0.1-0.75 mg/kg body weight, e.g. 0.1-0.4 mg/kg body weight) per 2-100 minutes, e.g. per 30-60 minutes, e.g. per 40-60 minutes, e.g. per 40 minutes.

According to a specific embodiment, ketamine (e.g. ketamine hydrochloride) is administered via intravenous (IV) administration at a dose of 0.5 mg/kg body weight per 40-60 minutes.

According to a specific embodiment, ketamine (e.g. ketamine hydrochloride) is administered via intravenous (IV) administration at a dose of 0.1-0.4 mg/kg body weight (e.g. 0.1 mg/kg, 0.2 mg/kg, 0.3 mg/kg, 0.4 mg/kg) per 40-60 minutes.

According to a specific embodiment, the therapeutically effective amount of ketamine (e.g. ketamine hydrochloride) comprises a dose of 0.1-1.0 mg/kg body weight (e.g. 0.1-0.75 mg/kg body weight, e.g. 0.1-0.4 mg/kg body weight) for intramuscular or subcutaneous administration.

According to a specific embodiment, ketamine (e.g. ketamine hydrochloride) is administered via intramuscular (IM) or subcutaneous administration at a dose of 0.1-1.0 mg/kg body weight (e.g. 0.1-0.5 mg/kg body weight, e.g. at a dose of 0.1 mg/kg body weight, 0.2 mg/kg body weight, 0.3 mg/kg body weight, 0.4 mg/kg body weight, 0.5 mg/kg body weight).

According to one embodiment, a therapeutically effective amount of ketamine for use via intramuscular (IM) or subcutaneous administration comprises a dose at least 10%, at least 20%, at least 30%, at least 40%, at least 50%%, at least 60%, at least 70%, at least 80%, or at least 90% lower than that of the Gold standard administered to psychiatric patients (e.g. a dose of 0.5-1.0 mg/kg body weight per injection).

According to one embodiment, a therapeutically effective amount of ketamine comprises a dose at least 50% lower than that of the Gold standard administered to psychiatric patients (e.g. a dose of 0.5-1.0 mg/kg body weight per injection).

According to one embodiment, a therapeutically effective amount of ketamine comprises a dose of no more than 0.75 mg/kg body weight, 0.5 mg/kg body weight, 0.4 mg/kg body weight, 0.3 mg/kg body weight, 0.2 mg/kg body weight, 0.1 mg/kg body weight per dose injection.

According to one embodiment, a therapeutically effective amount of ketamine comprises a dose of no more than 0.25 mg/kg body weight per dose injection.

According to a specific embodiment, the therapeutically effective amount of ketamine (e.g. ketamine hydrochloride) comprises a dose of 0.1-500 mg (e.g. 0.1-400 mg, e.g. 0.1-300 mg, e.g. 0.1-200 mg, e.g. 0.1-100 mg, e.g. 0.1-50 mg, e.g. 0.1-25 mg, e.g. 0.1-10 mg, e.g. 0.1-5 mg, e.g. 0.1-1 mg, e.g. 1-350 mg, e.g. 1-250 mg, e.g. 1-150 mg, e.g. 1-100 mg, e.g. 1-50 mg, e.g. 1-10 mg, e.g. 1-7.5 mg, e.g. 1-5 mg, e.g. 1-2.5 mg, e.g. 5-10 mg, e.g. 5-7.5 mg, e.g. 10-350 mg, e.g. 10-250 mg, e.g. 10-150 mg, e.g. 10-100 mg, e.g. 10-50 mg, e.g. 10-25 mg, e.g. 50-250 mg, e.g. 50-150 mg, e.g. 50-100 mg, e.g. 50-75 mg, e.g. 25-50 mg) for inhalation or intranasal administration.

According to a specific embodiment, ketamine (e.g. ketamine hydrochloride) is administered via intranasal spray (e.g. nebulized ketamine) at a dose of e.g. 0.1-500 mg, e.g. 0.1-400 mg, e.g. 0.1-300 mg, e.g. 0.1-200 mg, e.g. 0.1-100 mg, e.g. 0.1-50 mg, e.g. 0.1-25 mg, e.g. 0.1-10 mg, e.g. 0.1-1 mg, e.g. 1-350 mg, e.g. 1-250 mg, e.g. 1-150 mg, e.g. 1-100 mg, e.g. 1-50 mg, e.g. 1-10 mg, e.g. 1-7.5 mg, e.g. 1-5 mg, e.g. 1-2.5 mg, e.g. 5-10 mg, e.g. 5-7.5 mg, e.g. 10-350 mg, e.g. 10-250 mg, e.g. 10-150 mg, e.g. 10-100 mg, e.g. 10-50 mg, e.g. 10-25 mg, e.g. 50-250 mg, e.g. 50-150 mg, e.g. 50-100 mg, e.g. 50-75 mg).

According to a specific embodiment, ketamine (e.g. ketamine hydrochloride) is administered via intranasal spray (e.g. nebulized ketamine) at a dose of 0.1-500 mg, e.g. at a dose of 0.5 mg, 1 mg, 2 mg, 2.5 mg, 3 mg, 4 mg, 5 mg, 7.5 mg, 10 mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 250 mg, 300 mg.

According to a specific embodiment, ketamine (e.g. ketamine hydrochloride) is administered via intranasal spray (e.g. nebulized ketamine) at a dose of 0.1-500 mg/kg body weight, 0.1-400 mg/kg body weight, 0.1-300 mg/kg body weight, 0.1-200 mg/kg body weight, e.g. 0.1-100 mg/kg body weight, e.g. 0.1-10 mg/kg body weight, e.g. 0.1-1 mg/kg body weight, e.g. 10-350 mg/kg body weight, e.g. 10-250 mg/kg body weight, e.g. 10-150 mg/kg body weight, e.g. 10-100 mg/kg body weight, e.g. 10-50 mg/kg body weight, e.g. 50-250 mg/kg body weight, e.g. 50-150 mg/kg body weight, or e.g. 50-100 mg/kg body weight.

According to a specific embodiment, ketamine (e.g. ketamine hydrochloride) is administered via intranasal spray (e.g. nebulized ketamine) at a dose of 0.1-0.5 mg/kg body weight, 0.5-1 mg/kg body weight, 1-2 mg/kg body weight, 1-5 mg/kg body weight, 1-10 mg/kg body weight, 5-10 mg/kg body weight, 10-20 mg/kg body weight, 20-30 mg/kg body weight, 30-40 mg/kg body weight, 40-50 mg/kg body weight, 50-60 mg/kg body weight, 60-70 mg/kg body weight, 70-80 mg/kg body weight, 80-90 mg/kg body weight, 90-100 mg/kg body weight, 100-125 mg/kg body weight, 125-150 mg/kg body weight, 150-175 mg/kg body weight, or 175-200 mg/kg body weight (e.g. 0.5 mg/kg body weight, 0.75 mg/kg body weight, 1 mg/kg body weight, 1.5 mg/kg body weight, 2 mg/kg body weight, 2.5 mg/kg body weight, 3 mg/kg body weight, 4 mg/kg body weight, 5 mg/kg body weight, 10 mg/kg body weight, 15 mg/kg body weight, 20 mg/kg body weight, 25 mg/kg body weight, 30 mg/kg body weight, 40 mg/kg body weight, 50 mg/kg body weight, 60 mg/kg body weight, 70 mg/kg body weight, 80 mg/kg body weight, 90 mg/kg body weight, 100 mg/kg body weight, 125 mg/kg body weight, 150 mg/kg body weight, 175 mg/kg body weight or 200 mg/kg body weight).

According to one embodiment, a therapeutically effective amount of ketamine for use via inhalation or intranasal administration comprises a dose at least 10%, at least 20%, at least 30%, at least 40%, at least 50%, at least 60%, at least 70%, at least 80%, or at least 90% lower than that of the Gold standard administered to psychiatric patients (e.g. a dose of 50-150 mg, e.g. 56-84 mg, per inhalation or intranasal administration, e.g. administered twice weekly).

According to one embodiment, a therapeutically effective amount of ketamine comprises a dose at least 50% lower than that of the Gold standard administered to psychiatric patients (e.g. a dose of 50-150 mg, e.g. 56-84 mg, per inhalation or intranasal administration, e.g. administered twice weekly).

According to one embodiment, a therapeutically effective amount of ketamine comprises a dose of no more than 2.5 mg, 5 mg, 10 mg, 15 mg, 25 mg, 50 mg, 55 mg, 75 mg, 80 mg, 100 mg or 125 mg per inhalation or intranasal administration.

According to one embodiment, the therapeutically effective amount of ketamine (e.g. ketamine hydrochloride) comprises a dose of 0.1-1000 mg (e.g. 0.1-900 mg, e.g. 0.1-800 mg, e.g. 0.1-700 mg, e.g. 0.1-600 mg, e.g. 0.1-500 mg, e.g. 0.1-400 mg, e.g. 0.1-300 mg, e.g. 0.1-200 mg, e.g. 0.1-100 mg, e.g. 0.1-50 mg, e.g. 0.1-10 mg, e.g. 0.1-5 mg, e.g. 0.1-1 mg, e.g. 1-1000 mg, e.g. 1-750 mg, e.g. 1-500 mg, e.g. 1-400 mg, e.g. 1-300 mg, e.g. 1-250 mg, e.g. 1-150 mg, e.g. 1-100 mg, e.g. 1-75 mg, e.g. 1-50 mg, e.g. 1-25 mg, 1-10 mg, e.g. 10-1000 mg, e.g. 10-750 mg, e.g. 10-500 mg, e.g. 10-400 mg, e.g. 10-300 mg, e.g. 10-250 mg, e.g. 10-150 mg, e.g. 10-100 mg, e.g. 10-75 mg, e.g. 10-50 mg, e.g. 10-25 mg, e.g. 25-50 mg, e.g. 50-500 mg, e.g. 50-400 mg, e.g. 50-300 mg, e.g. 50-250 mg, e.g. 50-150 mg, e.g. 50-100 mg, e.g. 50-75 mg) for oral administration (e.g. per dosing occasion).

According to a specific embodiment, ketamine (e.g. ketamine hydrochloride) is administered via an oral route of administration at a dose of e.g. 10-500 mg, e.g. 10-20 mg, e.g. 20-30 mg, e.g. 30-40 mg, e.g. 40-50 mg, e.g. 50-60 mg, e.g. 60-70 mg, 70-80 mg, 80-90 mg, 90-100 mg, 100-125 mg, 125-150 mg, 150-175 mg, e.g. 175-200 mg, 200-225 mg, 225-250 mg, e.g. 250-300 mg, 300-350 mg, 350-400 mg, 400-450 mg or 450-500 mg (e.g. per dosing occasion).

According to a specific embodiment, ketamine (e.g. ketamine hydrochloride) is administered via an oral route of administration at a dose of 0.1-500 mg, e.g. at a dose of 0.1 mg, 0.5 mg, 1 mg, 2 mg, 2.5 mg, 3 mg, 4 mg, 5 mg, 7.5 mg, 10 mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 250 mg, 300 mg, 400 mg or 500 mg (e.g. per dosing occasion).

According to one embodiment, the therapeutically effective amount of ketamine (e.g. ketamine hydrochloride) for use via oral administration comprises a dose of 0.1-50 mg/kg body weight (e.g. 0.1-0.25 mg/kg body weight, 0.1-0.5 mg/kg body weight, 0.5-1 mg/kg body weight, 1-1.5 mg/kg body weight, 1.5-2 mg/kg body weight, 2-3 mg/kg body weight, 3-4 mg/kg body weight, 4-5 mg/kg body weight, 5-6 mg/kg body weight, 6-7 mg/kg body weight, 7-8 mg/kg body weight, 8-9 mg/kg body weight, 9-10 mg/kg body weight, 10-15 mg/kg body weight, 15-20 mg/kg body weight, 20-30 mg/kg body weight, 30-40 mg/kg body weight, 40-50 mg/kg body weight).

According to one embodiment, a therapeutically effective amount of ketamine for use via oral administration comprises a dose at least 10%, at least 20%, at least 30%, at least 40%, at least 50%, at least 60%, at least 70%, at least 80%, or at least 90% lower than that of the Gold standard administered to psychiatric patients (e.g. a dose of 100-250 mg per oral administration, e.g. administered 2-3 times per week).

According to one embodiment, a therapeutically effective amount of ketamine comprises a dose at least 50% lower than that of the Gold standard administered to psychiatric patients (e.g. a dose of 100-250 mg per oral administration, e.g. administered 2-3 times per week).

According to one embodiment, a therapeutically effective amount of ketamine comprises a dose of no more than 5 mg, 10 mg, 15 mg, 25 mg, 50 mg, 75 mg, 100 mg, 125 mg, e.g. 150 mg, 175 mg, 200 mg or 225 mg per oral administration (e.g. per dosing occasion).

The doses described herein above for ketamine are for intravenous, intramuscular, subcutaneous, oral, inhalation or intranasal modes of administration. Adjustment of the doses can be made for other routes of administration. Such determinations are well within the capability of one of skill in the art especially in view of the disclosure provided.

Ketamine can be commercially obtained from any of various sources, such as but not limited to, Pfizer (e.g. ketamine hydrochloride), Johnson & Johnson/Janssen (e.g. SPRAVATO® (esketamine)), Seelos Therapeutics (e.g. intranasal racemic ketamine (SLS-002)), and Bexson Biomedical.

According to one embodiment, a therapeutically effective amount of retigabine (ezogabine) is an amount capable of increasing the efficacy of ketamine or reducing ketamine's dosage for alleviation of symptoms of psychiatric disorders, such as depression-related disorders.

According to one embodiment, a therapeutically effective amount of retigabine (ezogabine) comprises a dose of 0.1-2000 mg/kg body weight, e.g. 0.1-1500 mg/kg body weight, e.g. 0.1-1000 mg/kg body weight, e.g. 0.1-500 mg/kg body weight, e.g. 0.1-100 mg/kg body weight, e.g. 0.1-50 mg/kg body weight, e.g. 0.1-25 mg/kg body weight, e.g. 0.1-10 mg/kg body weight, e.g. 0.1-5 mg/kg body weight, e.g. 0.5-500 mg/kg body weight, e.g. 0.5-100 mg/kg body weight, e.g. 0.5-50 mg/kg body weight, e.g. 5-500 mg/kg body weight, e.g. 5-100 mg/kg body weight, e.g. 5-50 mg/kg body weight, e.g. 10-50 mg/kg body weight, e.g. 10-25 mg/kg body weight, e.g. 50-100 mg/kg body weight, e.g. 100-200 mg/kg body weight, e.g. 200-300 mg/kg body weight, e.g. 300-400 mg/kg body weight, e.g. 400-500 mg/kg body weight, e.g. 500-600 mg/kg body weight, e.g. 600-700 mg/kg body weight, e.g. 700-800 mg/kg body weight, e.g. 900-1000 mg/kg body weight, e.g. 1000-1500 mg/kg body weight, or e.g. 1500-2000 mg/kg body weight.

According to one embodiment, the therapeutically effective amount of retigabine (ezogabine) is calculated per daily dose. For example, the therapeutically effective amount of retigabine (ezogabine) may comprise a dose of 0.1 mg/day to 2000 mg/day, 0.1 mg/day to 1000 mg/day, 0.1 mg/day to 500 mg/day, 0.1 mg/day to 100 mg/day, 0.1 mg/day to 50 mg/day, 0.1 mg/day to 10 mg/day, 0.5 mg/day to 2000 mg/day, e.g. 0.5 mg/day to 1500 mg/day, e.g. 0.5 mg/day to 1200 mg/day, e.g. 0.5 mg/day to 1000 mg/day, e.g. 5 mg/day to 1500 mg/day, e.g. 5 mg/day to 1200 mg/day, e.g. 10 mg/day to 500 mg/day, e.g. 10 mg/day to 100 mg/day, e.g. 50 mg/day to 1500 mg/day, e.g. 50 mg/day to 1200 mg/day, e.g. 100 mg/day to 1200 mg/day, e.g. 200 mg/day to 1200 mg/day, e.g. 300 mg/day to 1200 mg/day, e.g. 500 mg/day to 1200 mg/day, e.g. 600 mg/day to 1200 mg/day or e.g. 600 mg/day to 1000 mg/day.

According to one embodiment, retigabine (ezogabine) is administered in daily doses (e.g. 1-5 times a day, e.g. 1-3 times a day, e.g. 3 times a day).

According to one embodiment, retigabine (ezogabine) is administered in daily starting doses which may be increased gradually during time to reach a daily full dose. According to the present invention, such a starting dose is in the range of 0.5 mg/day to 500 mg/day, e.g. 0.5 mg/day to 400 mg/day, e.g. 0.5 mg/day to 300 mg/day, e.g. 0.5 mg/day to 150 mg/day, e.g. 0.5 mg/day to 75 mg/day, e.g. 0.5 mg/day to 50 mg/day, e.g. 0.5 mg/day to 25 mg/day or e.g. 0.5 mg/day to 10 mg/day.

According to one embodiment, the starting dose of retigabine (ezogabine) is lower or equal to 1800 mg/day, 1500 mg/day, 1200 mg/day, 900 mg/day, 600 mg/day, e.g. 500 mg/day, e.g. 400 mg/day, e.g. 300 mg/day, e.g. 200 mg/day, e.g. 100 mg/day. According to a specific embodiment, the starting dose of retigabine (ezogabine) is lower or equal to 300 mg/day (e.g. 100 mg every 8 hours).

According to one embodiment, the starting dose can be increased by 150 mg/day (e.g. 50 mg every 8 hours) at 1-week intervals.

According to one embodiment, a daily full dose of retigabine (ezogabine) which can be used according to the present invention may be in the range of 0.5 mg/day to 2000 mg/day, e.g. 0.5 mg/day to 1500 mg/day, e.g. 0.5 mg/day to 1200 mg/day, e.g. 0.5 mg/day to 600 mg/day, e.g. 5 mg/day to 1200 mg/day, e.g. 5 mg/day to 600 mg/day, e.g. 50 mg/day to 1200 mg/day, e.g. 50 mg/day to 600 mg/day, e.g. 100 mg/day to 1200 mg/day, e.g. 100 mg/day to 600 mg/day, e.g. 200 mg/day to 1200 mg/day, e.g. 300 mg/day to 1200 mg/day, e.g. 600 mg/day to 1200 mg/day.

According to a specific embodiment, the daily full dose of retigabine (ezogabine) is lower or equal to 1800 mg/day (e.g. 600 mg 3 times a day), 1500 mg/day (e.g. 500 mg 3 times a day), 1200 mg/day (e.g. 400 mg 3 times a day), e.g. 900 mg/day (e.g. 300 mg 3 times a day), 600 mg/day (e.g. 200 mg 3 times a day), 300 mg/day (e.g. 100 mg 3 times a day) or 150 mg/day (e.g. 50 mg 3 times a day).

According to a specific embodiment, the daily full dose of retigabine (ezogabine) is lower or equal to 1800 mg/day (e.g. 600 mg every 8 hours), 1500 mg/day (e.g. 500 mg every 8 hours), 1200 mg/day (e.g. 400 mg every 8 hours), e.g. 900 mg/day (e.g. 300 mg every 8 hours), 600 mg/day (e.g. 200 mg every 8 hours), 300 mg/day (e.g. 100 mg every 8 hours) or 150 mg/day (e.g. 50 mg every 8 hours).

It will be appreciated that the dose of retigabine (ezogabine) per day may be calculated per kg body weight (e.g. mg/kg body weight).

According to a specific embodiment, the therapeutically effective amount of retigabine (ezogabine) comprises a dose of 0.5-2000 mg administered orally (e.g. 0.5-1800 mg, 0.5-1500 mg, 0.5-1200 mg, 0.5-1000 mg, 0.5-900 mg, 0.5-800 mg, 0.5-700 mg, 0.5-600 mg, 0.5-500 mg, 0.5-400 mg, 0.5-300 mg, 0.5-200 mg, 0.5-150 mg, 0.5-100 mg, 0.5-90 mg, 0.5-80 mg, 0.5-70 mg, 0.5-60 mg, 0.5-50 mg, 0.5-40 mg, 0.5-30 mg, 0.5-20 mg, 0.5-10 mg or 0.5-25 mg).

According to a specific embodiment, the therapeutically effective amount of retigabine (ezogabine) comprises a dose of 0.5 mg, 1 mg, 5 mg, 10 mg, 25 mg, 50 mg, 75 mg, 100 mg, 150 mg, 200 mg, 250 mg, 300 mg, 400 mg, 500 mg, 600 mg, 700 mg, 800 mg, 900 mg, 100 mg, 1200 mg, 1500 mg, 1800 mg or 2000 mg administered orally.

The doses described herein above for retigabine (ezogabine) are for oral mode of administration. Adjustment of the doses can be made for other routes of administration (e.g. intravenous, intramuscular, intranasal, inhalation etc.). Such determinations are well within the capability of one of skill in the art especially in view of the disclosure provided.

Retigabine (ezogabine) can be commercially obtained from any of various sources, such as but not limited to, GlaxoSmithKline (under the trade names Trobalt® and Potiga®), Alomone labs (e.g. D-23129, Ezogabine), and Tocris Bioscience.

According to one embodiment, the methods of the invention may be further affected by administering to the subject an additional medicament (or any combination of medicaments) for the treatment of psychiatric disorders.

Exemplary medicaments for the treatment of a psychiatric disorder (e.g. depression-related disorders) which may be used in accordance with the present teachings include, but are not limited to, selective serotonin reuptake inhibitors (SSRIs), serotonin-norepinephrine reuptake inhibitors (SNRIs), noradrenergic and specific serotonergic antidepressants (NaSSAs), norepinephrine (noradrenaline) reuptake inhibitors (NRIs), norepinephrine-dopamine reuptake inhibitors, selective serotonin reuptake enhancers, norepinephrine-dopamine disinhibitors, tricyclic antidepressants (e.g. Imipramine), monoamine oxidase inhibitors (MAOIs).

According to one embodiment, treating a psychiatric disorder (e.g. depression-related disorders) may be further affected by administering to the subject an additional medicament (or any combination of medicaments) for the treatment of the psychiatric disorder or depression-related disorder including but not limited to, lithium (e.g. Lithium carbonate, Lithium citrate, Lithium sulfate), antipsychotic medicaments (e.g. typical antipsychotics and atypical antipsychotics, as detailed hereinabove), mood stabilizer medicaments (e.g. Valproic acid (VPA, Valproate), minerals, anticonvulsants, antipsychotics) and anti-depressants. Additional medicaments which may be used in accordance with the present teachings are described in detail hereinabove.

According to one embodiment, an efficient treatment (e.g. psychiatric disorder treatment such as anti-depressant/mood disorder treatment) is determined by assessing the patient's well-being, and additionally or alternatively, by subjecting the subject to behavioral tests, MRI or any other method known to one of skill in the art.

For in vivo therapy, the composition (e.g., NMDA receptor antagonist and KCNQ channel activator) is administered to the subject per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the molecule accountable for the biological effect (e.g. NMDA receptor antagonist and KCNQ channel activator).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, sublingual, rectal, transmucosal, transdermal, especially transnasal, intranasal, ocular, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient, e.g. local injection into a diseased tissue or in close proximity thereto.

According to a specific embodiment, the pharmaceutical composition is for an oral mode of administration.

According to a specific embodiment, the pharmaceutical composition is for a sublingual mode of administration.

According to a specific embodiment, the pharmaceutical composition is for a transmucosal mode of administration.

According to a specific embodiment, the pharmaceutical composition is for a transdermal mode of administration.

Conventional approaches for drug delivery to the central nervous system (CNS) include: neurosurgical strategies (e.g., intracerebral injection or intracerebroventricular infusion); molecular manipulation of the agent in an attempt to exploit one of the endogenous transport pathways of the BBB; pharmacological strategies designed to increase the lipid solubility of an agent (e.g., conjugation of the therapeutic molecules to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Methods for drug delivery behind the BBB include intracerebral implantation (such as with needles) and convection-enhanced distribution. Mannitol can be used in bypassing the BBB.

Likewise, mucosal (e.g., nasal) administration can be used to bypass the BBB.

According to a specific embodiment, the pharmaceutical composition is for an inhalation mode of administration.

According to a specific embodiment, the pharmaceutical composition is for an intranasal mode of administration.

Intranasal administration may be used for delivery of therapeutic agents to the central nervous system (CNS). The delivery occurs through the olfactory epithelium which is situated at the upper posterior part of the nasal cavity. The neurons of the olfactory epithelium project into the olfactory bulb in the brain hence enable a direct connection between the brain and the external environment. The transfer of drugs into the brain is thought to occur by either slow inner olfactory nerve cells transport or by a faster transfer along the perineural space surrounding the olfactory nerve cells into the cerebrospinal fluid in the brain. It is considered a non-invasive administration and allows large molecules that do not cross the BBB access to the CNS. This route of administration reduces systemic exposure and thus unwanted systemic side effects. Delivery from the nose to the CNS typically occurs within minutes and does not require the drug to bind to any receptor or axonal transport.

According to a specific embodiment, the composition is for intrathecal (IC), intracerebroventricular (ICV), ocular, or intravenous (IV) administration, where the composition will allow passage through the blood brain barrier (BBB).

According to one embodiment, the pharmaceutical composition is administered via intrathecal administration i.e. into the spinal canal, or into the subarachnoid space so that it reaches the cerebrospinal fluid (CSF).

According to one embodiment, the pharmaceutical composition is administered via an ocular mode of administration.

According to one embodiment, the pharmaceutical composition is administered via an intracerebroventricular (ICV) mode of administration, i.e. by injection directly into the cerebrospinal fluid in cerebral ventricles.

According to one embodiment, the pharmaceutical composition is administered via an intravenous (IV) mode of administration.

According to one embodiment, the pharmaceutical composition is administered via an intramuscular (IM) mode of administration.

According to one embodiment, the pharmaceutical composition is administered via a subcutaneous mode of administration.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount.

Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. NMDA receptor antagonist and KCNQ channel activator) effective to prevent, alleviate or ameliorate symptoms of a disorder (e.g., psychiatric disorder such as a depression-related disorder) or prolong the survival of the subject being treated.

According to an embodiment of the present invention, administration of the NMDA receptor antagonist (e.g. ketamine) and KCNQ channel activator (e.g. retigabine (ezogabine)) has an anti-depressant effect.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide sufficient plasma levels of the active ingredient to induce or suppress the biological effect (minimal effective concentration, MEC). The MEC will vary for each preparation, but can be estimated from in vitro data. Dosages necessary to achieve the MEC will depend on individual characteristics and route of administration.

Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. Such determinations are well within the capability of one of skill in the art especially in view of the disclosure provided.

The amount of a composition to be administered will, of course, be dependent on the subject being treated, the severity of the affliction, the manner of administration, the judgment of the prescribing physician, etc. The dosage and timing of administration will be responsive to a careful and continuous monitoring of the individual changing condition.

It will be appreciated that animal models exist by which the therapeutic molecules of the present invention may be tested prior to human treatment. For example, animal models of depression, stress, anxiety such as learned helplessness model (LH), chronic mild stress (CMS) model, social defeat stress (SDS) model and maternal deprivation model and sleep deprivation model may be used. For example, animal models of bipolar disease include, for example, transgenic mice with neuron-specific expression of mutant Polg (D181A) [as taught by Kato et al., Neuroscience and Biobehavioral Reviews (2007) 6 (31):832-842, incorporated herein by reference], as well as the well-established mania rat models of Amphetamine-induced hyperactivity [taught e.g. in U.S. Pat. No. 6,555,585], incorporated by reference, may be used.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, there is provided an article of manufacture comprising an NMDA receptor antagonist and a KCNQ channel activator.

According to a specific embodiment, the article of manufacture comprises a ketamine and a retigabine (ezogabine).

According to one embodiment, the NMDA receptor antagonist (e.g. ketamine) and the KCNQ channel activator (e.g. retigabine (ezogabine)) are in a co-formulation.

According to one embodiment, the NMDA receptor antagonist (e.g. ketamine) and the KCNQ channel activator (e.g. retigabine (ezogabine)) are in separate formulations.

According to one embodiment, the NMDA receptor antagonist (e.g. ketamine) and the KCNQ channel activator (e.g. retigabine (ezogabine)) may be included in the article of manufacture in a single or in separate packagings.

It will be appreciated that the therapeutic compositions of the invention may comprise, in addition to the NMDA receptor antagonist (e.g. ketamine) and the KCNQ channel activator (e.g. retigabine (ezogabine)), other known medications for the treatment of psychiatric disorders (e.g. depression-related disorders) such as, but not limited to, selective serotonin reuptake inhibitors (SSRIs), serotonin-norepinephrine reuptake inhibitors (SNRIs), noradrenergic and specific serotonergic antidepressants (NaSSAs), norepinephrine (noradrenaline) reuptake inhibitors (NRIs), norepinephrine-dopamine reuptake inhibitors, selective serotonin reuptake enhancers, norepinephrine-dopamine disinhibitors, tricyclic antidepressants (e.g. Imipramine), monoamine oxidase inhibitors (MAOIs). These medications may be included in the article of manufacture in a single or in separate packagings.

According to one embodiment, the therapeutic composition of the invention comprises, in addition to the NMDA receptor antagonist (e.g. ketamine) and the KCNQ channel activator (e.g. retigabine (ezogabine)), a medicament or any combination of medicaments, including but not limited to, lithium (e.g. Lithium carbonate, Lithium citrate, Lithium sulfate), antipsychotic medicaments (e.g. typical antipsychotics and atypical antipsychotics, as detailed below), mood stabilizer medicaments (e.g. Valproic acid (VPA, Valproate), minerals, anticonvulsants, antipsychotics) and anti-depressants.

Exemplary typical antipsychotic medicaments which may be used in accordance with the present teachings, include but are not limited to, Low potency medicaments: Chlorpromazine (Largactil, Thorazine), Chlorprothixene (Truxal), Thioridazine (Mellaril), Mesoridazine and Levomepromazine; Medium potency medicaments: Loxapine (Loxapac, Loxitane), Molindone (Moban), Perphenazine (Trilafon) and Thiothixene (Navane); High potency medicaments: Haloperidol (Haldol, Serenace), Fluphenazine (Prolixin), Droperidol, Zuclopenthixol (Clopixol), Flupentixol (Depixol), Prochlorperazine and Trifluoperazine (Stelazine). In addition, Prochlorperazine (Compazine, Buccastem, Stemetil) and Pimozide (Orap) may be used.

Exemplary atypical antipsychotic medicaments (also referred to as second generation antipsychotics) which may be used in accordance with the present teachings, include but are not limited to, Amisulpride (Solian), Aripiprazole (Abilify), Asenapine (Saphris), Blonanserin (Lonasen), Bitopertin (RG1678), Brexpiprazole (OPC-34712), Carpipramine (Prazinil), Clocapramine (Clofekton), Clozapine (Clozaril), Cariprazine (RGH-188), Iloperidone (Fanapt), Lurasidone (Latuda), LY2140023, Melperone (Buronil), Mosapramine (Cremin), Olanzapine (Zyprexa), Paliperidone (Invega), Perospirone (Lullan), Pimavanserin (ACP-103), Quetiapine (Seroquel), Remoxipride (Roxiam), Risperidone (Risperdal), Sertindole (Serdolect), Sulpiride (Sulpirid), Vabicaserin (SCA-136), Ziprasidone (Geodon), Zotepine (Nipolept) and Zicronapine (Lu 31-130).

Exemplary mood stabilizers which may be used in accordance with the present teachings, include but are not limited to, minerals (e.g. lithium); anticonvulsant mood stabilizers including Valproic acid (Depakine), divalproex sodium (Depakote), and sodium valproate (Depacon, Epilim), Lamotrigine (Lamictal), Carbamazepine (Tegretol), Oxcarbazepine (Trileptal), Topiramate (Topamax), Riluzole (Rilutek) and Gabapentin (Neurontin); antipsychotics (as described above); and food supplements (e.g. omega-3 fatty acids).

Exemplary anti-depressants which may be used in accordance with the present teachings, include but are not limited to, Selective serotonin reuptake inhibitors (SSRIs, such as Citalopram, Escitalopram, Fluoxetine, Fluvoxamine, Paroxetine and Sertraline); Serotonin-norepinephrine reuptake inhibitors (SNRIs, such as Desvenlafaxine, Duloxetine, Milnacipran and Venlafaxine); Noradrenergic and specific serotonergic antidepressants (such as Mianserin and Mirtazapine); Norepinephrine (noradrenaline) reuptake inhibitors (NRIs, such as Atomoxetine, Mazindol, Reboxetine and Viloxazine); Norepinephrine-dopamine reuptake inhibitors (such as Bupropion); Selective serotonin reuptake enhancers (such as Tianeptine); Norepinephrine-dopamine disinhibitors (NDDIs such as Agomelatine); Tricyclic antidepressants (including Tertiary amine tricyclic antidepressants and Secondary amine tricyclic antidepressants); and Monoamine oxidase inhibitor (MAOIs).

According to one embodiment, the anti-depressant drug comprises selective serotonin reuptake inhibitors (SSRI), tricyclic antidepressants and noradrenaline reuptake inhibitors (NRI).

According to a specific embodiment, the anti-depressant drug comprises selective serotonin reuptake inhibitors (SSRI).

It will be appreciated that additional non-pharmaceutical therapeutic strategies may be employed in combination with the present teachings, including but not limited to, clinical psychology, electroconvulsive therapy, involuntary commitment, light therapy, psychotherapy, transcranial magnetic stimulation and cognitive behavioral therapy.

It is expected that during the life of a patent maturing from this application many relevant NMDA receptor antagonists and the KCNQ channel activators will be developed and the scope of the terms “NMDA receptor antagonist” and “KCNQ channel activator” are intended to include all such new molecules a priori.

As used herein the term “about” refers to ±10%.

The terms “comprises”, “comprising”, “includes”, “including”, “having” and their conjugates mean “including but not limited to”.

The term “consisting of “means” including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Animals and Animal Housing

All experiments were performed in accordance with the European Communities' Council Directive 2010/63/EU. All protocols were approved by the Ethics Committee for the Care and Use of Laboratory Animals of the government of Upper Bavaria, Germany and by the Institutional Animal Care and Use Committee (IACUC) of the Weizmann Institute of Science (Rehovot, Israel). CD-1 (ICR) and C57BL/6N male mice aged between 7-11 weeks old were used for all experiments. Mice were bred in the animal facility of the Max Planck Institute of Biochemistry (Martinsried, Germany) and group housed (4 to 5 mice per cage). Mice were kept in individually ventilated cages (IVC; 30 cm×16 cm×16 cm; 501 cm²), serviced by a central airflow system (Tecniplast, IVC Green Line-GM500), according to institutional guidelines. IVCs had sufficient bedding and nesting material as well as a wooden tunnel for environmental enrichment. Animals were maintained under a pathogen-free, temperature-controlled environment (23±1° C.) and constant humidity (55±10%) on a 12 hour light-dark cycle (lights on at 7 am) with food and water provided ad libitum, at the Max Planck Institute of Psychiatry (Munich, Germany).

The Social Box Arenas

The behavior of mice was studied in specialized “Social Box” (SB) arenas, designed for automated tracking of individual and group behaviors, as described previously [S. Anpilov et al., Neuron (2020) 107: 644-655 e647]. Each arena housed four male mice that had been grouped together at the time of weaning. The SB consisted of an open 60×60 cm box and included the following objects: a covered nest, an open small nest, an S-shaped wall, two water bottles, two feeders and two elevated ramps. Food and water were available ad libitum. The arenas were illuminated at ca. 2 lux during the dark phase (12 hours) and at ca. 200 lux (using light-emitting diodes) during the light phase (12 hours). The fur of all mice was painted using four different hair dyes under mild isoflurane anesthesia. The period under anesthesia was typically no longer than 10 minutes and the mice were left to recover for several days before the start of the experiment. A color-sensitive camera (Manta G-235C, Allied-Vision) was placed 1 μm above the arena and recorded the mice during the dark phase. Mouse trajectories were automatically tracked offline using a combination of both a specially written software in Matlab (Mathworks) [Y. Shemesh et al., (Bio-protocol, 2020), chap. https://en(dot)bio-protocol(dot)org/prep207], and the markerless pose estimation software DeepLabCut (DLC, v. 2.1.10) [A. Mathis et al., Nat Neurosci (2018) 21: 1281-1289]. On day 1, animals were transferred to the SB, for a total of 4 days of baseline (four dark and three light periods). On day 5, mice were removed from the SB and were administered either ketamine (10 mg/kg body weight (BW)) or saline intraperitoneally, followed by a Forced Swim Test (FST) 30 minutes later. All mice were subsequently returned into a clean SB for response monitoring over the following 36 hours (2 dark and 1 light period). Normalized SB behavioral readouts (a total of 228 features) were summarized in 3 hour time bins for the Baseline and Response days (Days 3-6). To account for baseline individual differences, the change in each readout was calculated for each time bin from the mean of the corresponding time bin over the Baseline days. The change values from the first 3 hour period of Day 5 (immediately following the injection and FST procedure) were used to train a supervised partial least squares-based classifier.

Characterization of the Mouse Behavioral Response to Ketamine

To gage the acute effects of pharmaceutical manipulations, the analysis of the SB tracks was limited to the first 3 hours of the dark phase (immediately following the FST, detailed below). Likewise, the tracks from the Baseline days 3 and 4 were limited to the corresponding segment of the dark phase. DLC was used to track three key points (the nose, the center of mass, and base of the tail) for each individual for the duration of these videos. Preprocessing of the trajectory data and summaries of behavioral readouts were performed using a set of custom R functions. 228 behavioral readouts (features) were extracted for each individual in each of three separate dark phases. The median of each feature over the two Baseline days was used to create a baseline assessment. Each feature was transformed within each stage/cohort combination to approximate a Gaussian distribution using a rank-based inverse normal transformation (Blom transform, rankNorm function in the RNOmni R package, v.1.0) [M. Z. (rdrr.io, R Package Documentation, 2019), chap. https://rdrr(dot)io/cran/RNOmni/]. The transformed values were used to calculate individual change scores (Response—Baseline) for each feature. The ketamine response score was developed using partial least squares discriminant analysis (PLS-DA), as implemented in the mixOmics package in R (v. 6.12.2) [F. Rohart, et al., PLoS Comput Biol (2017) 13: e1005752]. The training dataset consisted of 64 individuals (48 received ketamine (10 mg/kg BW) and 16 received saline) and the input data consisted of the SB behavioral change scores combined with all FST behavioral readouts.

Forced Swim Test (FST)

Mice were placed in a 2 L glass beaker filled with 1.5 L of water at room-temperature (23±1° C.) to a height of 14 cm so that the mouse could neither escape nor touch the bottom. The test lasted 6 minutes and was later analyzed by an experienced experimenter, blind to the experimental group. Time spent immobile and time spent struggling during the test were scored.

Chronic Social Defeat Stress Paradigm

C57BL/6N and Nex-Cre-Ai9 males (7 weeks old) were exposed to the CSDS paradigm for 10 consecutive days, as previously described [J. P. Lopez et al., Sci Adv (2021) 7]. Experimental mice were introduced daily into the home cage of a dominant CD-1 resident mouse, which rapidly recognized and attacked the intruders. To avoid serious injuries, the subordinate mouse was separated immediately after being attacked by the CD-1 aggressor. After the physical encounter, mice were separated by a perforated metal partition, allowing the mice to keep continuous sensory but not physical contact for the next 24 hours. Every day, for a total of 10 days, mice were defeated by another unfamiliar, CD-1 mouse, to exclude a repeated encounter throughout the experiment. Defeat encounters were randomized, with variations in starting time (between 8:00 a.m. and 6:00 p.m.) to decrease the predictability to the stressor and minimize habituation effects. Control mice were single-housed, in the same room as the stressed mice, throughout the course of the experiment. All animals were handled daily and weighed every 2-3 days. Coat state was scored on a scale of 0 to 3 according to the following criteria: (0) No wounds, well-groomed and bright coat, and clean eyes; (1) no wounds, less groomed and shiny coat, or unclean eyes; (2) small wounds, and/or dull and dirty coat, and not clear eyes; (3) extensive wounds, or broad piloerection, alopecia, or crusted eyes. End point and tissue collection were performed in the morning (8:00 a.m.) and 24 hours after the last social defeat session (day 11). This was done to capture the cumulative effects of chronic stress, rather than the acute effects of the last defeat session. For end point, all mice were deeply anesthetized with isoflurane and target tissues were quickly dissected for molecular experiments. Cardiac blood was collected for the assessment of basal CORT levels (discussed in detail hereinbelow). Adrenal glands were dissected from Fat and Weighed. The Brains were Collected for Dissection of the Ventral Hippocampus.

Home Cage Activity

The home cage activity was measured with the Mouse-E-Motion infrared-detecting devices (Infra-e-motion, Germany). Mice were single housed in fresh cages, and a metal food tray was employed to hold the devices in place. The base bedding was kept, but extra nesting materials that could conceal the animal were removed. The readout lasted 2 days (post-injection) during which time the animals were not disturbed. Locomotor activity was detected in 5-minute increments and averaged by the hour. The final analysis was applied to the first 60-minute period and the 48 hours (2 days), post injection.

Single-Cell RNA Sequencing

Tissue Dissociation

All single-cell procedures were performed as previously described [J. P. Lopez et al., Sci Adv (2021) 7]. Mice were anesthetized lethally using isoflurane and perfused with cold PBS in order to get rid of undesired blood cells in target tissues. Brains were quickly dissected and immediately transferred to ice-cold oxygenated artificial cerebral spinal fluid (aCSF) and kept in the same solution during dissection and dissociation. The aCSF was oxygenated throughout the experiment with a mixture of 5% CO₂ in O₂. Sectioning of the brain was performed using a VT1200/S Leica vibratome. A 1000 μm thick slice (approximately −2.46 mm Bregma to −3.52 mm Bregma) was obtained from each brain and the ventral hippocampus was manually dissected under a stereo microscope (M205C, Leica). The ventral hippocampus was dissociated using the Papain dissociation system (Worthington) for 35 minutes at 37° C. in a shaking water bath, following the manufacturer's instructions. All cell suspensions were filtered with 30 μm filters (Partec) and kept in cold aCSF.

Cell Capture, Library Preparation, and High-Throughput Sequencing

Single cells were resuspended in ice-cold aCSF and prepared for single-cell labeling and capture using the iCELL8 Single-Cell System (Takara Bio), according to the manufacturer's recommendations. Cells were stained with DAPI (for live cells) and propidium iodide (for dead cells) for 10 minutes and dispensed in the loading plate. Each iCell8 chip was loaded with cells from two different mice (ketamine and saline-treated). Following microfluidic separation, iCell8 chips were imaged using the built-in fluorescence microscope, snap-frozen using dry ice and stored at −80° C. until library preparation. Based on fluorescence labeling, only wells containing live single cells were selected for library preparation. All wells containing dead cells or multiplets were excluded. Libraries were prepared according to the manufacturers' guidelines using the iCell8 Chip and Reagent Kit, in-chip RT-PCR amplification chemistry (Wafergen, Takara Bio), Nextera XT DNA Library Preparation Kit, and Nextera XT Index Kit (Illumina). Libraries were assessed using a High Sensitivity DNA Analysis Kit for the 2100 Bioanalyzer (Agilent) and KAPA Library Quantification kit for Illumina (KAPA Biosystems), and sequencing was performed paired-end with 26 nt/100 nt configuration on an Illumina HiSeq4000 system at the Helmholtz Zentrum Sequencing Core Facility (Munich, DE).

Pre-Processing, Quality Control and Normalization

For the initial quality check, FastQC [A. Simon and R. chap. www(dot)bioinformatics(dot)babraham(dot)ac(dot)uk/projects/fastqc.%202010] was employed before demultiplexing the cells by barcode using Jemultiplexer version 1.0.6 [C. Girardot, et al., BMC Bioinformatics (2016) 17: 419] requiring a perfect match of the sequence. Adaptor trimming was performed using cutadapt version 1.11 [M. Martin, EMBnet J (2011) 17]. To extract and collapse unique molecular identifiers (UMIs) from the sequencing data, UMI-tools version 0.5.4 [T. Smith, et al., Genome Res (2017) 27, 491-499] modules extract and dedup (deduplication mode per gene) were used. Alignment and subsequent quantification after UMI deduplication were carried out by Salmon version 0.8.2 [R. Patro, et al., Nat Methods (2017) 14: 417-419], specific settings included noLengthCorrection, perTranscriptPrior and noEffectiveLengthCorrection to accommodate tag sequencing. As transcriptomic reference, known RefSeq transcripts with the addition of mitochondrial genes were used.

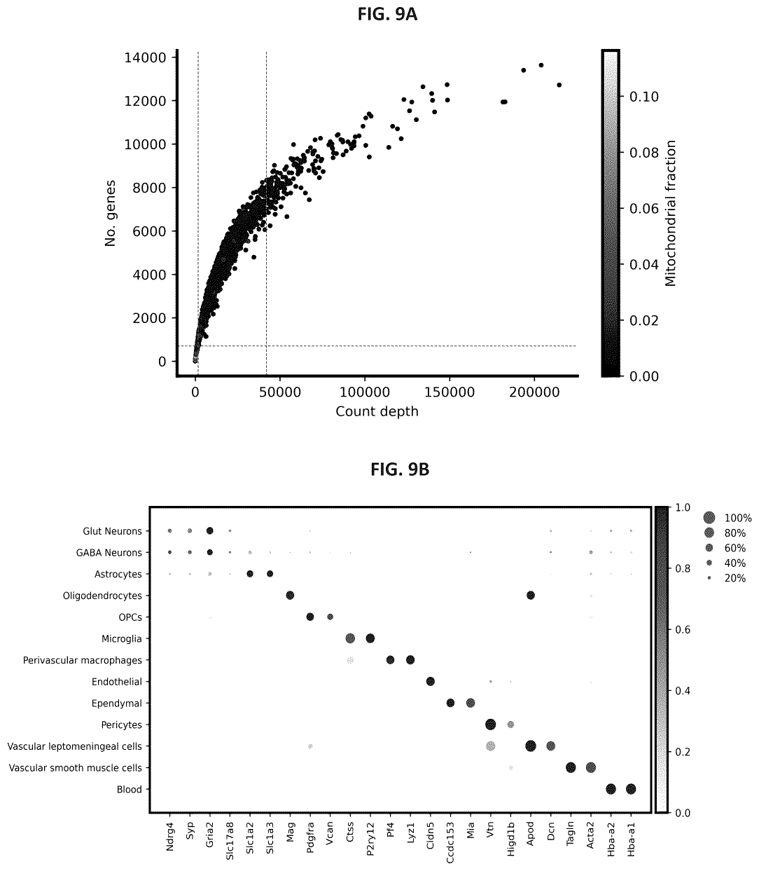

Data processing and analysis was done in SCANPY [F. A. Wolf et al., Genome Biol (2018) 19: 15] (version 1.5.1) using AnnData as a data format (version 0.7.5). After considering the joint distribution of count depth, the number of genes expressed, and mitochondrial (MT) read fraction per sample, cells with more than 42,000 counts, with fewer than 700 genes expressed, and with 20% or more reads aligned to mitochondrial genes were filtered out. Furthermore, genes that were measured in fewer than 20 cells were also removed from the dataset. Quality control plots can be found in FIG. 9A. This left a dataset of 5,013 cells and 20,670 genes. To assess which genes might be affected by ambient RNA signal, the gini coefficient was computed per gene (https://github(dot)com/oliviaguest/gini; 2021) and the zero expression rate (also called dropout rate) per gene. The gini coefficient assesses how evenly spread the expression of a gene was. The present inventors reasoned that potential ambient genes were those genes that had a lower dropout rate than would be expected given how evenly they were expressed. To quantify this, a linear model was fitted using numpy's polyfit function to predict dropout rate from the gini coefficient. Potential ambient genes were defined as genes that had a lower actual dropout rate than predicted from the linear fit by a margin of over 1.5 times the standard deviation of the regression coefficient. This resulted in 9 potentially ambient genes (Apoe, Hbb-bs, Trf, Ptgds, Hba-a2, Tcf4, Cst3, Plp1, Hba-a1), which were ignored when assigning labels and were not considered in the differential expression evaluation.

Normalization and batch correction were performed in a two-stage process. First, size factors for each cell were computed using scran pooling [A. T. Lun et al., Genome Biol (2016) 17: 75] (scran R package, version 1.18.3). These size factors were used to normalize the data for the selection of highly variable genes. In a second step, the scran pooling size factors, a batch covariate, and the number of UMI per gene were used as a covariate in a negative binomial regression model fit to the count data following [C. Mayer et al., Nature (2018) 555: 457-462]. The Pearson residuals from this fit were used as a quantification of the expression values. Scran pooling was performed as implemented in the computeSumFactors function. A pre-clustering of the data was passed to facilitate the fitting procedure (using log CPM+1, Euclidean distance on the top 50 PCs to construct a k-nearest neighbor graph with k=15, and louvain clustering at resolution 1; see clustering methods below), and set the min_mean parameter to 0.1. In a second step, a regularized negative binomial model was fit to each gene as described in [C. Mayer (2018), supra]. For each gene i, the following model was fit:

log(E(Yᵢⱼ))=β₀+β₁βⱼ+β₂Uⱼ+β₃Sⱼ

Here E(Yᵢⱼ) denotes the expected value of the UMI count distribution of gene i across cells j, Bⱼ denotes the batch covariate of cell j, Uⱼ denotes the number of UMIs per expressed gene in cell j, and Sⱼ represents the scran pooling size factor for cell j. β denotes a regression coefficient. The iCell8 chip identifier was used as a batch covariate. As described by sctransform normalization [C. Hafemeister et al., Genome Biol (2019) 20: 296], this model was regularized by fitting a Poisson regression with the above model per gene to obtain an empirical dispersion parameter, 0, then a regularized dispersion parameter was obtained by fitting a loess fit to the mean-variance relationship across genes. As the batch covariate was included in the above model, the Pearson residuals of the regularized negative binomial regression could be used as a batch-corrected expression unit for downstream analysis.

Clustering, Sub-Clustering, Marker Gene Detection, and Cluster Annotation

Graph-based clustering was performed on the computed KNN graph using the python implementation (version 0.6.1) of the Louvain algorithm in Scanpy. As a starting point, a Louvain clustering was performed at a resolution of 1. For each cluster, marker genes were determined by applying Welch's t-test (as implemented in Scanpy's rank_genes_groups function with default parameters) between the cells in the cluster and all other cells. Differential expression testing for marker gene detection was performed on the log-scran normalized, non-batch-corrected expression values as recommended by published best practices [M. D. Luecken and F. J. Theis, Mol Syst Biol (2019) 15: e8746]. Clusters were annotated using a set of literature-derived markers [A. Zeisel et al., Cell (2018) 174: 999-1014 e1022]. Marker-based annotation was performed by comparing the mean, scaled expression of all cells in a cluster, both on the level of individual markers and of marker sets associated with a cell identity label. Clusters that could not be distinctly annotated were merged (e.g., astrocyte subclusters, and glutamatergic neuronal subclusters), and further subclustering was performed at a louvain resolution of 0.4 to distinguish glutamatergic and GABAergic neurons, vascular cells from pericytes, and perivascular macrophages from microglia. Two populations without distinct marker gene signatures were removed as low quality cells. After discarding these populations, 13 annotated clusters were left.

Differential Expression Analysis

Differential expression analysis per cell identity cluster was performed via the limma package (version 3.46.0). Specifically, the limma-trend pipeline was used, replacing CPM normalization with the scran pooling normalization described above. For each annotated cluster, the following linear model was fit to all genes expressed in at least 10% of the cells in that cluster:

Yᵢⱼ˜1+Bⱼ+Cⱼ

Here, Cⱼ represents the condition label (ketamine or saline) of cell j, Bⱼ represents the batch covariate label of the cell, and 1 denotes that an intercept was fit. The iCell8 chip identifier was used as batch covariate. This above model was fit using log-normalized data (from scran pooling normalization), and an empirical Bayes prior was used to fit the gene-wise variances via limma's eBayes function. Differentially expressed genes were filtered out if expression was <1 in both conditions. Multiple testing correction was performed using the Benjamini-Hochberg method.

Generation of Nex-Cre-Ai9 mice

Conditional transgenic mice expressing tdTomato in glutamatergic neurons of the forebrain (Nex-Cre;Ai9) were generated by crossing homozygous Nex-Cre mice [described in S. Goebbels et al., Genesis (2006) 44: 611-621] with homozygous Ai9 mice [Gt(ROSA)26Sortm9(CAG-tdTomato)Hze] [described in L. Madisen et al., Nat Neurosci (2010) 13: 133-140].

Fluorescence-Activated Cell Sorting (FACS) Analysis of Live Cells

For FACS analysis, single-cell suspensions from the ventral hippocampus of mice treated with ketamine or a saline control were prepared as described earlier (see tissue dissociation, above). Four samples per treatment were analyzed as biological replicates. The procedure followed was described previously [I. Y. Buchsbaum et al., EMBO Rep (2020) 21: e48204]. FACS analysis was performed with a FACS Melody (BD) in BD FACS Flow TM medium, with a nozzle diameter of 100 m. Debris and cell aggregates were gated out by forward scatter (FSC)-side scatter (SSC). Single cells were gated by FSC-W/FSC-A. Gating strategies for TdTomato+ cells were selected using single-cell suspensions of ventral hippocampus from wild-type C57BL/6N mice. Examples of the gating strategy are shown in FIGS. 9A-B.

Reverse Transcription and Quantitative Real-Time Polymerase Chain Reaction (qRT-PCR)

Messenger RNA (mRNA) samples were extracted using the miRNeasy kit according to the manufacturer's instructions (Qiagen). Quantification of mRNA levels (bulk) was carried out using quantitative real-time PCR (qRT PCR). Total RNA was reverse transcribed using the High-Capacity cDNA Reverse Transcription Kit (Applied Biosystems). Real-time PCR reactions were run in triplicate using the ABI QuantStudio6 Flex Real-Time PCR System and data was collected using the QuantStudio Real-Time PCR software (Applied Biosystems). Expression levels were calculated using the standard curve, absolute quantification method. The geometric mean of the endogenous expressed genes Rp113 and Gapdh were used to normalize the data. The list of primers used is provided in Table 1, below.

TABLE 1
List of primers

| | | SEQ |
|---|---|---|
| | | ID |
| Primer Name | Sequence | NO |
| Clqc_Fwd | GTGCACCTGAACCTCAACCT | 1 |
| Clqc_Rev | CGGGAAACAGTAGGAAACCA | 2 |
| Clasp1_Fwd | ACAGCTCTTTGCGTGGAGTT | 3 |
| Clasp1_Rev | GCCATCCTGCCTCCTTCTAT | 4 |
| Cldn5_Fwd | CTGGACCACAACATCGTGAC | 5 |
| Cldn5_Rev | GCCGGTCAAGGTAACAAAGA | 6 |
| E2f1_Fwd | CAACTGCAGGAGAGTGAGCA | 7 |
| E2f1_Rev | CCATCTGTTCTGCAGGGTCT | 8 |
| Gapdh_Fwd | CCATCACCATCTTCCAGGAG | 9 |
| | CGAG | |
| Gapdh_Rev | GATGGCATGGACTGTGGTCA | 10 |
| | TGAG | |
| Hsp90ab1_ | GCATGAAGGAGACCCAGAAG | 11 |
| Fwd | | |
| Hsp90ab1_ | CACTGAGACCAGGCTCTTCC | 12 |
| Rev | | |
| Hspa8_Fwd | ATGTTGCTTTCACGGACACA | 13 |
| Hspa8_Rev | GGGCCAGTGCTTCATATCAG | 14 |
| Ilf2_Fwd | GCTCTTCTGATGCTACGGTGA | 15 |
| Ilf2_Rev | GAGAAGCGTTCTCTTCAAACC | 16 |
| Kcnq2_Fwd | TACCGCAAGCTGCAGAATTT | 17 |
| Kcnq2_Rev | CCCCTCAGAGCTCTTCTCGT | 18 |
| Kcnq3_Fwd | TGCCTGGTACATAGGCTTCC | 19 |
| Kcnq3_Rev | AGACGTCCTTCCCAGGTTTT | 20 |
| Mog_Fwd | GCAGGTCTCTGTAGGCCTTG | 21 |
| Mog_Rev | GTGCAGCCAGTTGTAGCAGA | 22 |
| Ndufa4_Fwd | AGGAGGTCCTGGGTGACTTT | 23 |
| Ndufa4_Rev | CAGTACCCCCTGCTCCAATA | 24 |
| Neurod6_Fwd | TGGAAAGGGTCAAGTTCAGG | 25 |
| Neurod6_Rev | GGTCTCTTGCCAATCCTCAG | 26 |
| Rpl13_Fwd | CACTCTGGAGGAGAAACGGA | 27 |
| | AGG | |
| Rpl13_Rev | GCAGGCATGAGGCAAACAGTC | 28 |
| Slc17a7_Fwd | CTGGGGTCCTTGTGCAGTAT | 29 |
| Slc17a7_Rev | AACAGGGTTCATGAGCTTGG | 30 |
| Slc32al_Fwd | CTGGAACGTGACAAATGCCA | 31 |
| Slc32al_Rev | CGGCGAAGATGATGAGGAAC | 32 |
| Snap25_Fwd | AAAAAGCCTGGGGCAATAAT | 33 |
| Snap25_Rev | CTCACCTGCTCCAGGTTCTC | 34 |
| TdTomato_ | GGCATTAAAGCAGCGTATCC | 35 |
| Fwd | | |
| TdTomato_ | CTGTTCCTGTACGGCATGG | 36 |
| Rev | | |

Primary Cell Culture

Primary hippocampal neurons were generated from E16.5 embryos using a standard primary neuron cell culture protocol previously described [K. G. Schraut et al., Eur JNeurosci (2021) 53: 390-401]. Briefly, dissected hippocampi were harvested in ice-cold dissection medium (HBSS, 7 mM HEPES, 2 mM L-glutamine, 500 U/ml penicillin-streptomycin, all Thermo Fisher Scientific). The tissue was incubated for 10 minutes in 0.25% Trypsin-EDTA with 8 mM HEPES (Thermo Fisher Scientific) in a water bath at 37° C. Tissue was washed three times with serum medium (DMEM, 10% FBS, Thermo Fisher Scientific). The cells were dissociated by titration using glass Pasteur pipets 10 in serum medium and filtered with a 70 tm cell strainer (Corning). Cell culture plates were coated overnight with 0.05 mg/ml poly-D-lysine mol wt 70,000-150,000 (Sigma Aldrich) in 0.15 M borate buffer (pH=8.5). Cells were seeded in 24-well plates at a density of 5×10⁴ cells in 1 ml growth medium (Neurobasal A medium, 1 x B27 supplement, 0.25×GlutaMAX, all Thermo Fisher Scientific) per well. All cultures were kept in a humidified incubator at 37° C. and 5% CO₂. Growth medium was renewed after 7 days of culture. For all experiments, mature twenty-one-day old primary neuronal cultures were used.

Ex Vivo Electrophysiological Recordings

Animals

CD-1 (ICR) male mice (5-weeks old) were purchased from Harlan Laboratories (Jerusalem, Israel). Mice were kept in groups of 4 or 5 animals per cage and were 7-8-week-old at the beginning of the experiment. Throughout the experiments, the animals were maintained in a temperature-controlled room (22±1° C.) and constant humidity (55±10%) on a 12 hour light-dark cycle (lights on at 7 am). Food and water were given ad libitum. All experimental protocols were approved by the Institutional Animal Care and Use Committee of the Weizmann Institute of Science. One week before the start of the experiment, the animals were single-housed and randomly assigned to the vehicle- or ketamine-treated group. Mice were handled following the same protocol used for the ScRNA seq experiments. Each animal received an intraperitoneal injection of 10 mg/kg BW ketamine (or saline for vehicle-treated mice) 30 minutes before being subjected to a 6 minute Forced Swim Test (see description of the FST, above). Mice were then returned to their home cage and left undisturbed for 36 hours before being used for electrophysiological recordings.

Brain Slices Preparation

Mice (vehicle- and ketamine-treated) were injected with pentobarbital (100 mg/kg BW i.p.) and perfused with carbogenated (95% O₂, 5% CO₂) ice-cold slicing solution containing (in mM): 2.5 KCl, 11 glucose, 234 sucrose, 26 NaHCO₃, 1.25 NaH₂PO₄, 10 MgSO₄, 2 CaCl₂); pH 7.4, 340 mOsm. After decapitation, 300 m-thick horizontal slices containing the ventral hippocampus were prepared in carbogenated ice-cold slicing solution using a vibratome (Leica VT 1200S) and allowed to recover for 20 minutes at 33° C. in carbogenated high osmolarity artificial cerebrospinal fluid (aCSF) (high-Osm) containing (in mM): 3.2 KCl, 11.8 glucose, 132 NaCl, 27.9 NaHCO₃, 1.34 NaH₂PO₄, 1.07 MgCl2, 2.14 CaCl₂); pH 7.4, 320 mOsm. Subsequently, slices were incubated for 40 minutes at 33° C. in carbogenated aCSF containing (in mM): 3 KCl, 11 glucose, 123 NaCl, 26 NaHCO₃, 1.25 NaH₂PO₄, 1 MgCl2, 2 CaCl₂); pH 7.4, 300 mOsm. Finally, slices were kept at room temperature (23-25° C.) in the same solution until use.

Patch-Clamp Recordings and M-Currents Isolation

CA1 pyramidal neurons were patched under visual guidance using infrared differential interference contrast (DIC) microscopy (BX51W1, Olympus) and an Andor Neo sCMOS camera (Oxford Instruments, Abingdon, UK). Borosilicate glass pipettes (BF100-58-10, Sutter Instrument, Novato, CA, USA) with resistances 4-6 MQ were pulled using a laser micropipette puller (P-2000, Sutter Instrument) and filled with intracellular solution (in mM: 135 potassium-gluconate, 4 KCl, 2 NaCl, 10 HEPES, 4 EGTA, 4 Mg-ATP, 0.3 Na₂-GTP, 10 phosphocreatine-Na₂, 280 mOsm kg-1, pH adjusted to 7.3 with KOH). Somatic whole-cell voltage-clamp recordings from CA1 pyramidal neurons (>1 GQ seal resistance, −70 mV holding potential) were performed using a Multiclamp 700B amplifier (Molecular Devices, San Jose, CA, USA). Data were acquired using pClamp 10.7 on a personal computer connected to the amplifier via a Digidata-1440 interface (sampling rate: 20 kHz; low-pass filter: 4 kHz), and analyzed with Clampfit 10.7 (all Molecular Devices). Data obtained with a series resistance >20 MQ were discarded.

All experiments were conducted at room temperature. In the recording chamber, slices were superfused with carbogenated aCSF (4-5 ml/min flow rate) containing 0.2 mM CdCl₂, 1 μM TTX, 10 μM ZD7288, and 4 mM 4− aminopyridine, to block voltage-dependent Cav, Nav, HCN, and Kvl channels, respectively. Synaptic activity was blocked with 10 μM NBQX-Na₂ (2,3-dioxo-6-nitro-1,2,3,4-tetrahydrobenzo[f]quinoxaline-7-sulfonamide disodium salt), 50 μM D-APV (D-2−amino-5-phosphonopentanoic acid) and 10 μM (−)-bicuculline methiodide. To isolate M-currents, the following protocol was applied to the recorded cell, modified from Nigro et al., 2014 [M. J. Nigro, P. et al., J Neurosci (2014) 34: 6807-6812]: (1) a 1 second step from the holding potential (−70 mV) to −10 mV was applied (to activate IM while inactivating most other voltage-gated currents); (2) a 1 second step to −50 mV (to elicit IM tail current); and (3) a 0.5 second step to −10 mV, before returning to the holding potential. This procedure was repeated every 10 seconds for 5 minutes as baseline.

To investigate the effect of ketamine treatment on KCNQ 2/3, the selective KCNQ 2/3 inhibitor, XE991 (40 μM), was applied and its effects was measured during the last 100 ms of the first step to −10 mV (Iss), and on the fast component of the tail current (Itail), after 10 minutes of application. NBQX, D-APV, TTX-citrate, (−)-bicuculline methiodide, 4− aminopyridine, XE991 [10,10-bis(4-pirinydilmethyl)-9(10H)−antracenone] were obtained from Alomone Labs, and ZD7288 from Tocris Bioscence. All the remaining chemicals were obtained from Sigma-Aldrich.

In Vitro Electrophysiological Recordings

Primary hippocampal neurons were generated from E16.5 embryos and cultured for 21 days, as described above (see Primary Cell Culture). Somatic whole-cell voltage-clamp recordings (>1 GQ seal resistance, <20 MQ series resistance, −70 mV holding potential, 2 kHz low-pass filter, 6 kHz sampling rate, 10 mV liquid junction potential correction) in saline or HNKet (10 μM, 24 hours)-treated cultures were performed at 25° C. using an EPC9 amplifier. Cells were superfused (2-3 ml/min) with carbogenated aCSF containing (in mM): 125 NaCl, 2.5 KCl, 25 NaHCO₃, 1.25 NaH₂PO₄, 2 CaCl₂), 1 MgCl2, 10 D-glucose, 5 4− aminopyridine, 0.001 TTX, and 0.02 ZD7288. Patch pipette solution consisted of (in mM): 130 K-gluconate, 5 NaCl, 2 MgCl₂, 0.5 EGTA, 10 HEPES, 2 Mg-ATP, 0.3 Na-GTP, 20 phosphocreatine, and 5 D-glucose. M-current measurements/analyses were conducted as described for slice recordings (see above).

Cloning and Validation of shRNA Constructs

The control shRNA scramble and Kcnq2 shRNA1 sequences were previously described [M. Valdor et al., Mol Pain (2018) 14: 1744806917749669]. The Kcnq2 shRNA2 sequence was designed using siRNA wizard software (Invitrogen). The shRNA sequences were synthesized in the pcDNA3 expression vector with the KpnI and BamHI restriction sites flanking the shRNA sequence (BioCat). Control shRNA (SEQ ID NO: 37):

Control shRNA (SEQ ID NO: 37):
ggtaccGATCCCACTACCGTTGTTATAGGTGT
TCAAGAGACACCTATAACAACGGTAGTTTTTT
TGggatcc
Kcnq2 shRNA1 (SEQ ID NO: 38):
ggtaccGATCCCGGTATTCGGTGTTGAGTACT
TCAAGAGAGTACTCAACACCGAATACCTTTTT
TGggatcc
Kong2 shRNA2 (SEQ ID NO: 39):
ggtaccGATCCCCGTGGTATTCGGTGTTGAGT
ACCAAGAGGTACTCAACACCGAATACCACGTT
TTTTGggatcc

A pAAV-H1-EFla-eGFP backbone was linearized using KpnI and BamHI restriction enzymes, opening up a region right after the H1 promoter. The shRNA fragments were digested with KpnI and BamHI and ligated into the pAAV-H1-EFla-eGFP backbone using T4 DNA Ligase according to the provided protocol (NEB). This generated the following four vectors, pAAV-H1-Ctrl-shRNA-EFla-eGFP, pAAV-H1-KCNQ-shRNA1-EFla-eGFP, and pAAV-H1-KCNQ-shRNA2-EFla-eGFP. All plasmids were checked for mutations by DNA sequencing. The constructs were validated in mouse neuroblastoma neuro2a (N2a) cells. These cells were maintained at 37° C. with 5% CO₂ in Minimum Essential Medium (MEM), lx Glutamax, supplemented with 1× non-essential amino acids, 1 mM sodium pyruvate, 100 U/ml penicillin, 100 μg/ml streptomycin and 10% FBS (Thermo Fisher Scientific). Cells were detached with trypsin and transfected using ScreenfectA (ScreenFect GmbH) according to the manufacturer's protocol and maintained for two days before analysis. For imaging, cells were fixed with 4% PFA-PBS solution and embedded with Fluoromount-G mounting medium containing DAPI (SouthernBiotech). Cells were imaged using an Axioplan 2 fluorescent microscope (Zeiss). For qPCR analysis, RNA was extracted and Kcnq2 expression was determined using qPCR (see reverse transcription and qPCR methods section).

Virus Production and Stereotactic Surgery

The production of rAAV particles was previously described [L. M. Fiori et al., Mol Psychiatry, (2020)]. The rAAVs were produced with capsids of serotypes 1/2. The number of viral genomic particles was determined using qPCR resulting in titers of 2-5×10¹² gp/ml. For viral injections, CD-1 mice were anesthetized with isoflurane and placed on a 37° C. heating pad in a stereotactic apparatus (TSE Systems). Pre-surgery, mice were given Novalgin (200 mg/kg BW) and Metacam (sub-cutaneous 0.5 mg/kg BW). During surgery, mice were continuously supplied with 2% v/v isoflurane in O₂ through inhalation. AAV virus was injected bilateral using a 33-gauge injection needle with a 5 μl Hamilton syringe coupled to an automated microinjection pump (World Precision Instruments). For molecular studies, 0.5 μl virus was injected at a rate of 0.1 μl/min. The injection coordinates were determined using the Franklin and Paxinos mouse brain atlas, from bregma: ML+/−3.2 mm bilateral; AP −3.2 mm; DV 3.5 mm. After injection the needle was retracted 0.01 mm and kept at the injection site for 1 min/0.1 μl of injected volume, followed by slow withdrawal. After surgery, the animals received Metacam for at least three days (intraperitoneal 0.5 mg/kg BW). After completion of the behavioral experiments, the injection sites were verified based on eGFP expression. Brains were fixed overnight with 4% paraformaldehyde-PBS followed by dehydration in 30% sucrose-PBS solution for at least 24 hours at 4° C. Brains were cut in 50 μm thick sections using a vibratome (HM 650 V, Thermo Scientific). Brain slices were embedded in DAPI containing Fluoromount-G mounting medium (SouthernBiotech). Slices were imaged with a VS120-S6-W slide scanner microscope (Olympus) or with a LSM800 confocal microscope (Zeiss).

Plasma CORT Measurements

Blood sampling was performed during end point (8:00 a.m.) by collecting trunk blood after decapitation. All blood samples were kept on ice and centrifuged at 4° C., and 10 μl of plasma was removed for measurement of CORT. All plasma samples were stored at −20° C. until CORT measurement. CORT concentrations were quantified by radioimmunoassay (RIA) using a CORT double antibody 1251 RIA kit (sensitivity: 25 ng/ml; MP Biomedicals Inc.) following the manufacturer's instructions. Radioactivity of the pellet was measured with a gamma counter (Wizard2 2470 Automatic Gamma Counter; Perkin Elmer). All samples were measured in duplicate, and the intra- and inter-assay coefficients of variation were both below 10%. Final CORT levels were derived from the standard curve.

Pharmacological Experiments

In Vitro Treatments

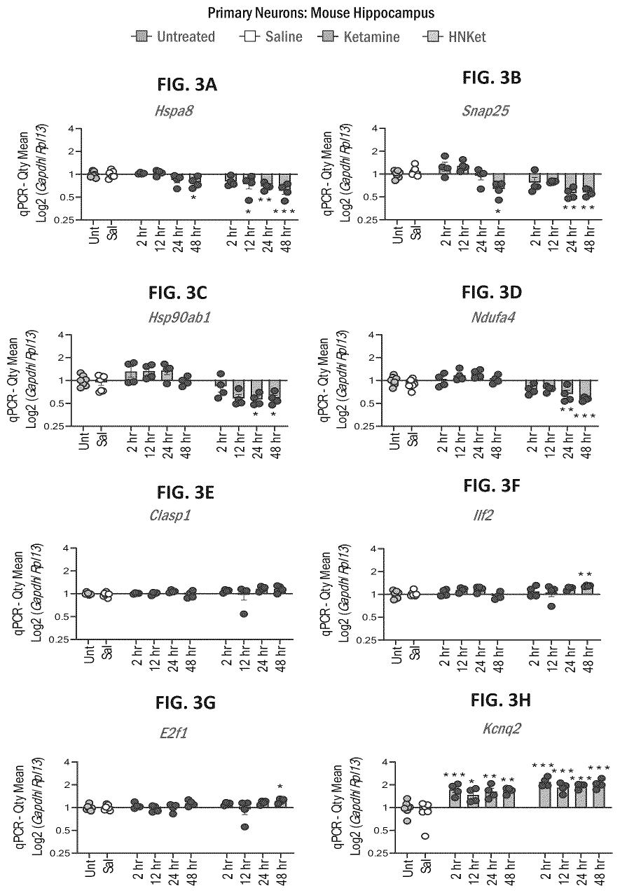

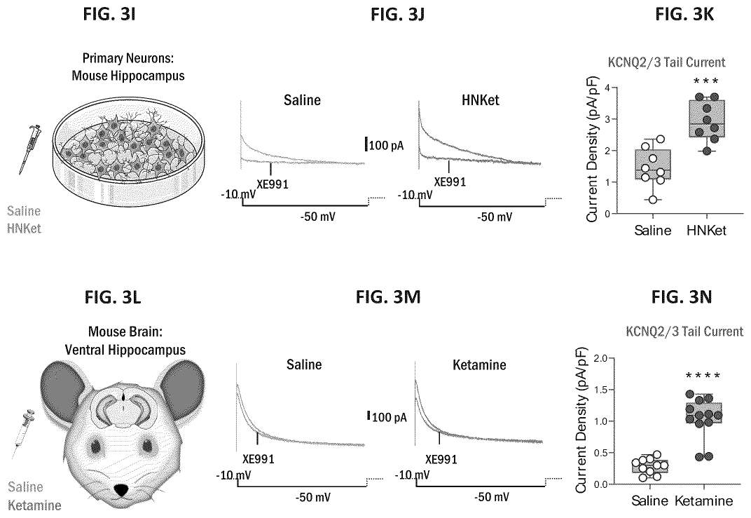

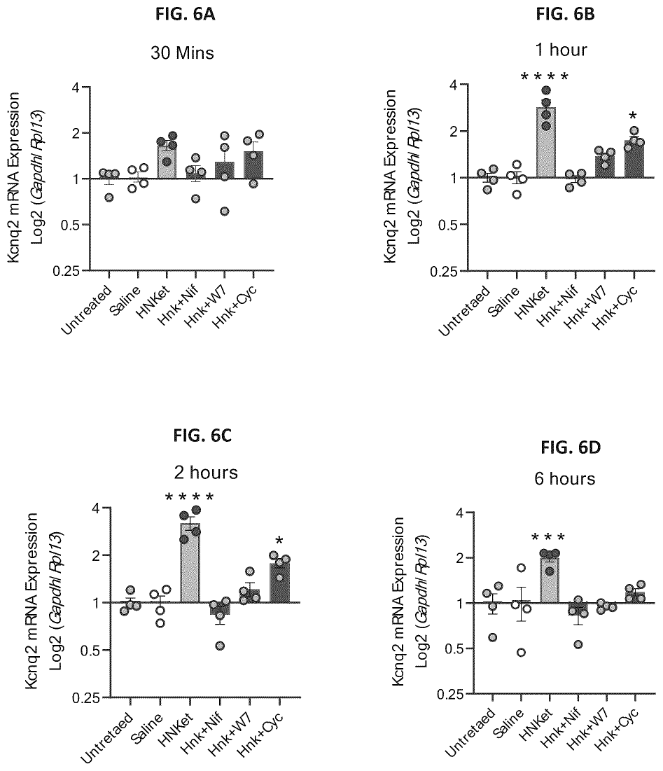

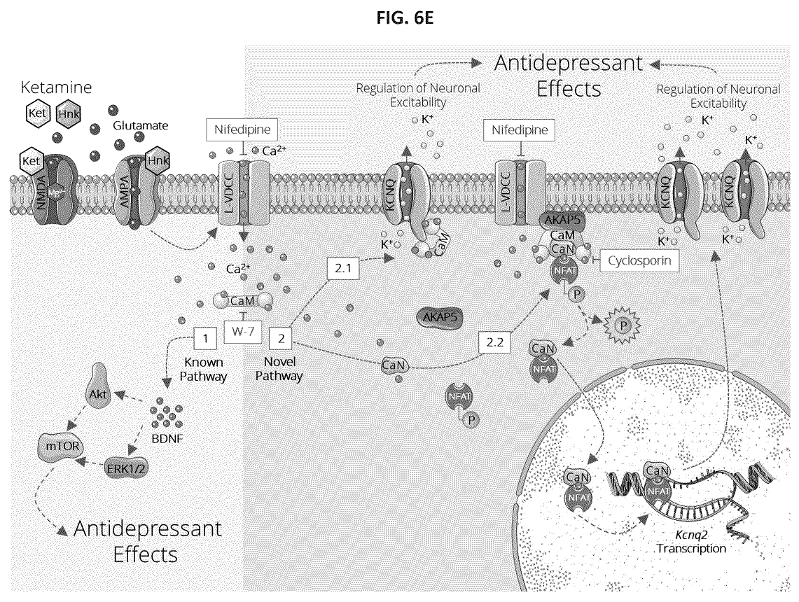

Primary hippocampal neurons were generated from E16.5 embryos and cultured for 21 days, as described above (see Primary Cell Culture). For experiment 1 (FIGS. 3A-K), neurons were treated with a saline solution, (R,S)-ketamine (10 μM) (Ketaset, Zoetis, Germany) or (2R,6R)-HNKet (10 μM) (Sigma, Cat #SML1873-25MG) for 0, 2, 12, 24 or 48 hours and compared to untreated controls. For experiment 2 (FIGS. 6A-D), neurons were stimulated with either a saline solution, (2R,6R)-HNKet (10 μM), or a combination of (2R,6R)-HNKet (10 μM) plus nifedipine (10 μM) (Tocris Bioscience, Cat #1075), W-7 hydrochloride (10 μM) (Tocris, Cat #0369), or cyclosporine-A (1 μM) (Tocris, Cat #1101) for 30 minutes, 1, 2, or 6 hours, and compared to untreated controls.

In Vivo Treatments

C57BL/6N and CD-1 (ICR) male mice aged 10 weeks old were used in these experiments. Ketamine hydrochloride (Ketaset, Zoetis, Germany) was diluted in 0.9% NaCl solution (saline) and administered i.p at 1, 5 or 10 mg/kg BW, depending on the experiment design. The KNCQ inhibitor, XE991 (Alomone labs; Cat #: X-100) was diluted in 5% DMSO and administered i.p at 1 or 3 mg/kg BW. The KCNQ activator, Retigabine (Alomone labs; Cat #: R-100), was diluted in 5% DMSO and administered i.p at 1 or 5 mg/kg BW. As control, mice were injected with a DMSO-saline solution (5% DMSO). For all experiments, mice were tested 30 minutes after injection in the FST or over a period of 36 hours after injection in the social boxes.

### Example 1

ScRNA-Seq Reveals Cell-Type Specific Molecular Signatures of Ketamine Treatment in the Ventral Hippocampus

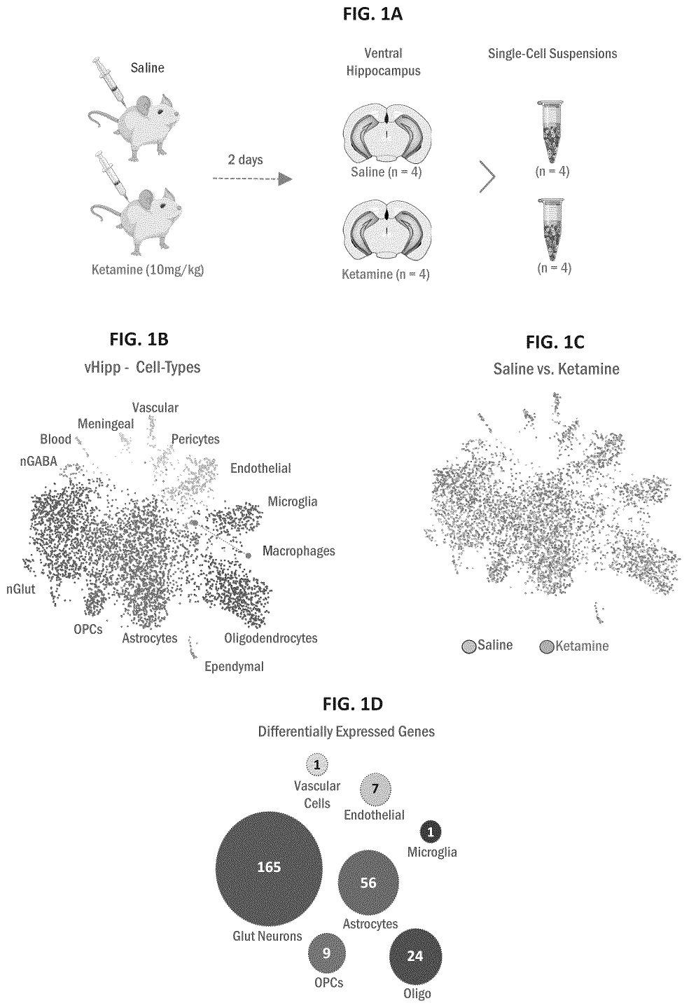

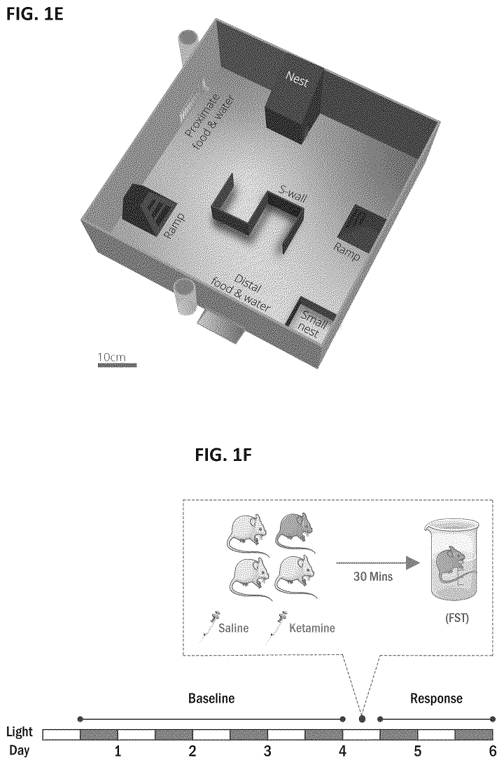

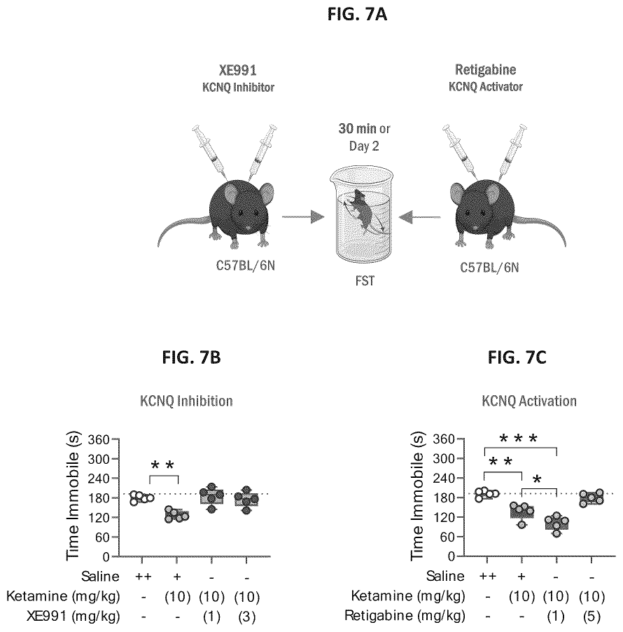

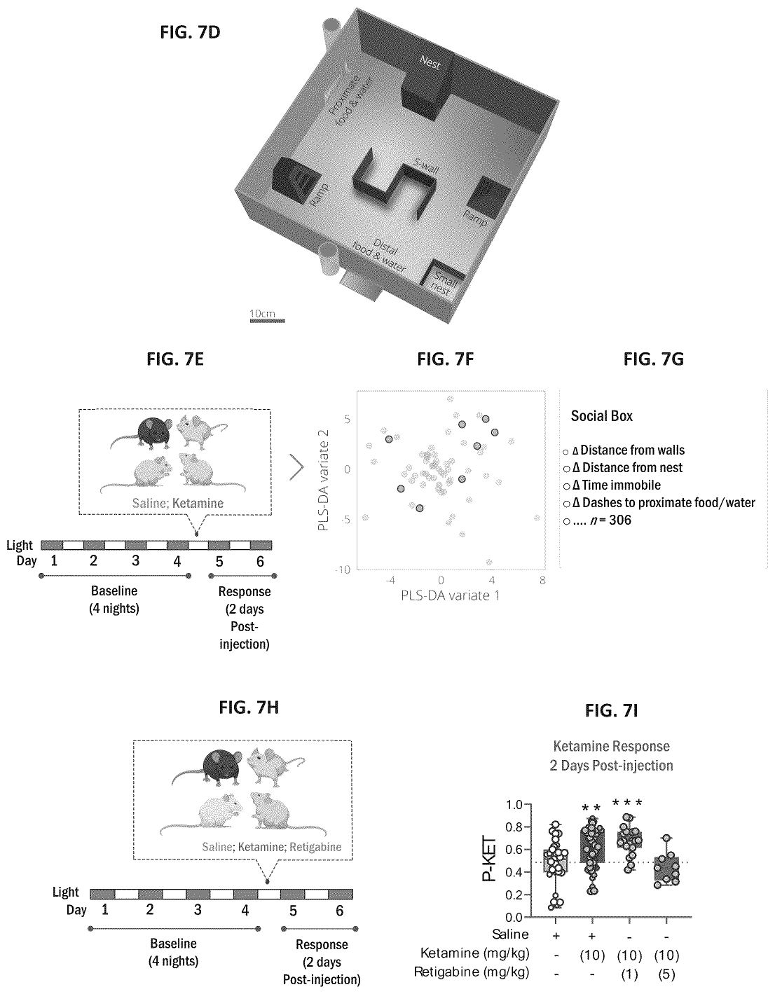

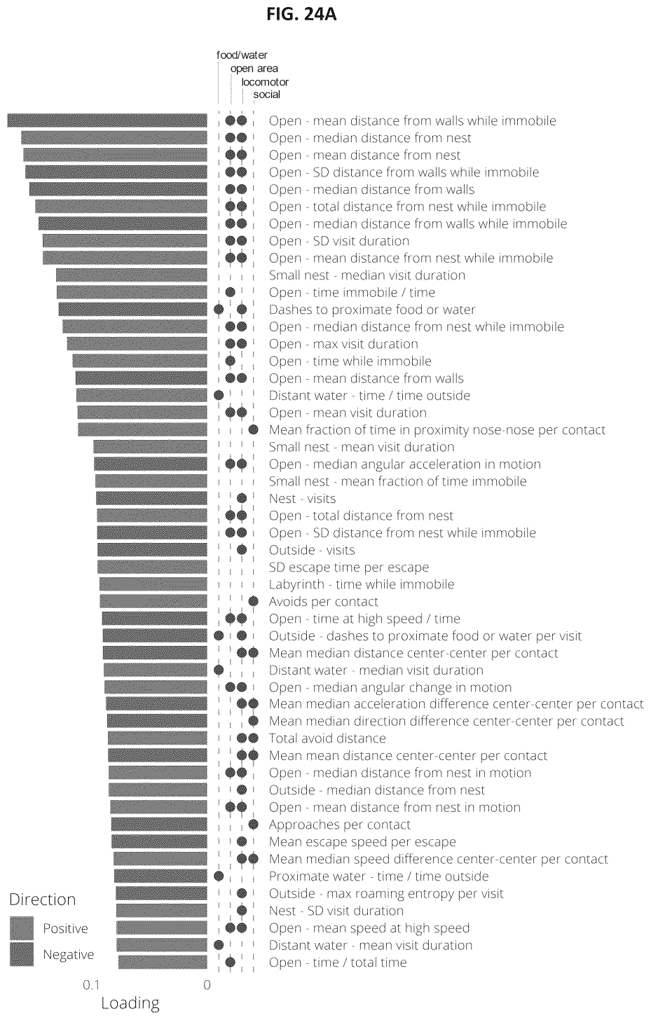

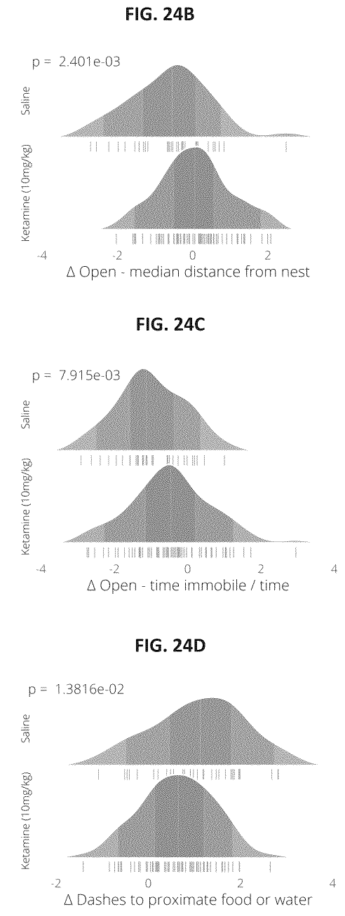

In order to perform an in-depth characterization of the behavioral and molecular response to ketamine in mice, a testing procedure was established that took advantage of both classical and a more recent and naturalistic approach for behavioral assessment in rodents [S. Anpilov et al. (2020) supra]. Adult male mice living in groups of four were introduced into the Social Box (SB) (FIG. 1E), an enriched housing environment, where they lived under continuous video observation for five days and six nights (FIG. 1F). The first four nights were used to establish individual and group baseline behaviors for all mice. Before the start of the dark phase on Day 5, mice were administered with either (R,S)-ketamine (10 mg/kg/body weight (BW)) or saline, intraperitoneally (i.p), followed by a Forced Swim Test (FST), a validated and commonly used test for evaluation of antidepressant efficacy in rodents [R. Yankelevitch-Yahav et al., J Vis Exp (2015)]. All mice were subsequently returned into a clean SB for response monitoring over the following two nights (36 hours). This procedure was performed on an initial cohort of sixty-four mice (16 groups), allowing assessments of individual differences in ketamine response and establishment of an analysis pipeline for the SB data. A description of the pipeline is provided above. Briefly, normalized SB behavioral readouts (a total of 306 features) were summarized in three-hourly time bins for the Baseline (Days 3-4) and Response days (Days 5-6). To account for baseline individual differences, the change in each readout for each time bin from the mean of the corresponding time bin over the Baseline days was calculated (FIGS. 7F-G). The change values from the first three-hour period of Day 5 (immediately following the injection-FST procedure) were used to train a supervised partial least squares-based classifier (PLSDA) (FIGS. 7F-G). This combination of methods allowed to augment the standard assessment for the short-term effects of ketamine (measured by the FST only) with a range of longer-term more ethologically relevant outcomes (by the SB) in a setup, which deliberately increases the heterogeneity of behavioral expression through environmental and social enrichment. A summary of loadings of behavioral readouts onto the PLSDA-based classifier of ketamine response in the SB can be found in FIGS. 24A-D. Following the SB response monitoring period (36 hours post-injection, i.e. 2 days), four mice from each condition were randomly selected (ketamine-treated vs. saline-controls), their brains were collected, and single-cell suspensions were prepared for molecular characterization using scRNA-seq (FIG. 1A).

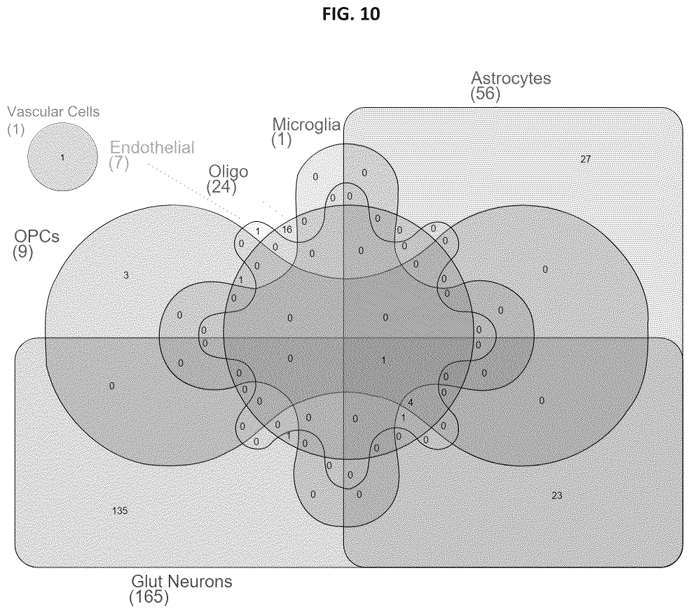

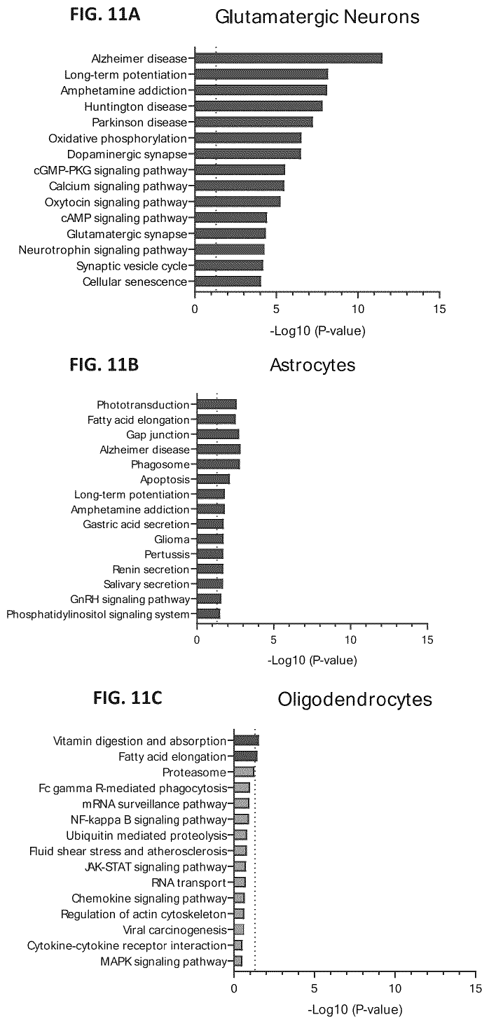

In order to investigate cell-type-specific molecular signatures of ketamine response, the transcriptome of single cells from the vHipp, a well-known region for ketamine antidepressant action in rodents and humans were sequenced [P. Zanos and T. D. Gould, Mol Psychiatry (2018) 23: 801-811]. Following best-practices [M. D. Luecken and F. J. (2019), supra], the identity of cell clusters were cataloged using Scanpy, a scalable toolkit for single-cell gene expression analysis [F. A. Wolf et al., (2018), supra]. After quality control (QC) analysis and pre-processing (FIGS. 9A-B), single cells were grouped according to their unique gene expression profiles using graph-based clustering and uniform manifold approximation and projection (UMAP) plots were used for visualization of clusters (FIGS. 1B-D). This unsupervised cluster analysis revealed a total of 13 cell clusters with distinct gene expression signatures (FIG. 1B). The identity of each cluster was determined based on the expression of well-established cell-type-specific markers from the literature [A. Zeisel et al., (2018), supra] (FIGS. 9A-B), as follows: glutamatergic neurons (nGlut), GABAergic neurons (nGABA), astrocytes, oligodendrocytes, oligodendrocyte progenitor cells (OPCs), microglia, macrophages, endothelial cells, ependymal cells, pericytes, vascular cells, meningeal cells, and blood cells (FIG. 1B). The relative cell type composition for each cluster was assessed by comparing the total number of cells from the ketamine and saline treated groups but no significant differences were found between groups (FIG. 1C, and Table 2A, below), suggesting no major changes in cell composition following ketamine treatment. Subsequently, differential expression analyses were performed to evaluate cell-type-specific molecular signatures of ketamine action. A total of 263 differentially expressed genes (DEGs) were identified in 7 of the 13 clusters, ranging from 1 to 165 DEGs per cell type (FIG. 1D and Table 2B, below). 31 of the 263 DEGs were found to be significantly dysregulated in more than 1 cluster, however 135 genes differentially expressed exclusively in glutamatergic neurons, 27 in astrocytes, 16 in oligodendrocytes, 3 in OPCs, 1 in endothelial cells, and 1 in vascular cells (FIG. 10 and Table 3, below). Additionally, a pathway enrichment analysis was performed for the three cell types with the largest DEGs, using Enrichr (Xie et al., Current Protocols (2021)). In glutamatergic neurons, this analysis revealed a large number of significant pathways involved in calcium signaling, synaptic function and plasticity and neurodevelopmental disorders (FIG. 11A). Astrocytes, showed an enrichment for fatty acid elongation, gap junction, phagosome activity and Alzheimer disease (FIG. 11B), while oligodendrocytes showed an enrichment for vitamin digestion, absorption, and fatty acid elongation (FIG. 11C).

TABLE 2A
Distribution of Single cells per cluster

| | | | | Saline | Ketamine |
|---|---|---|---|---|---|
| Cell Type | Saline | Ketamine | Total | (%) | (%) |
| nGlut | 852 | 843 | 1695 | 50.27% | 49.73% |
| Astrocytes | 772 | 802 | 1574 | 49.05% | 50.95% |
| Oligodendrocytes | 344 | 288 | 632 | 54.43% | 45.57% |
| Endothelial | 135 | 160 | 295 | 45.76% | 54.24% |
| Microglia | 138 | 128 | 266 | 51.88% | 48.12% |
| OPCs | 113 | 116 | 229 | 49.34% | 50.66% |
| Pericytes | 43 | 44 | 87 | 49.43% | 50.57% |
| Ependymal | 28 | 31 | 59 | 47.46% | 52.54% |
| Meningeal cells | 21 | 25 | 46 | 45.65% | 54.35% |
| nGABA | 21 | 19 | 40 | 52.50% | 47.50% |
| Vascular cells | 13 | 27 | 40 | 32.50% | 67.50% |
| Blood | 11 | 19 | 30 | 36.67% | 63.33% |
| Macrophages | 15 | 5 | 20 | 75.00% | 25.00% |
| Total | 2506 | 2507 | 5013 | 49.99% | 50.01% |

TABLE 2B
List of differentially expressed genes (DEGs) per cell type

| # | Gene | Cell Type | DEG # | Change | logFC | adj.P.Val | Mean-Sal | Mean-Ket |
|---|---|---|---|---|---|---|---|---|
| 1 | Ilf2 | Glut Neurons | 1 | Up | 0.379 | 1.24E−17 | 1.142 | 1.495 |
| 2 | D10Wsu102e | Glut Neurons | 2 | Up | 0.260 | 3.31E−08 | 2.733 | 3.002 |
| 3 | Eef1akmt3 | Glut Neurons | 3 | Up | 0.276 | 3.70E−08 | 1.445 | 1.701 |
| 4 | Zfp488 | Glut Neurons | 4 | Up | 0.236 | 5.25E−06 | 1.092 | 1.333 |
| 5 | Cyb5r4 | Glut Neurons | 5 | Up | 0.215 | 8.92E−06 | 0.789 | 1.027 |
| 6 | Dpp10 | Glut Neurons | 6 | Up | 0.216 | 1.69E−05 | 1.104 | 1.344 |
| 7 | Zfp369 | Glut Neurons | 7 | Up | 0.207 | 2.80E−05 | 0.814 | 1.005 |
| 8 | Acad9 | Glut Neurons | 8 | Up | 0.210 | 1.27E−04 | 1.149 | 1.372 |
| 9 | Il17ra | Glut Neurons | 9 | Up | 0.252 | 1.35E−04 | 1.157 | 1.490 |
| 10 | Hsd3b2 | Glut Neurons | 10 | Up | 0.193 | 2.22E−04 | 0.958 | 1.162 |
| 11 | Saa3 | Glut Neurons | 11 | Up | 0.193 | 2.35E−04 | 1.319 | 1.494 |
| 12 | Fgd4 | Glut Neurons | 12 | Up | 0.144 | 3.23E−04 | 2.192 | 2.397 |
| 13 | Nvl | Glut Neurons | 13 | Up | 0.148 | 1.22E−03 | 0.882 | 1.070 |
| 14 | Mrpl48 | Glut Neurons | 14 | Up | 0.180 | 1.33E−03 | 0.864 | 1.060 |
| 15 | Kcnj16 | Glut Neurons | 15 | Up | 0.227 | 2.28E−03 | 0.751 | 1.052 |
| 16 | Clasp1 | Glut Neurons | 16 | Up | 0.274 | 2.28E−03 | 1.217 | 1.557 |
| 17 | E2f1 | Glut Neurons | 17 | Up | 0.186 | 4.33E−03 | 0.854 | 1.125 |
| 18 | Nav2 | Glut Neurons | 18 | Up | 0.163 | 5.80E−03 | 0.985 | 1.176 |
| 19 | Mdga2 | Glut Neurons | 19 | Up | 0.158 | 8.49E−03 | 0.986 | 1.191 |
| 20 | Fut8 | Glut Neurons | 20 | Up | 0.166 | 1.11E−02 | 1.181 | 1.297 |
| 21 | Dhx16 | Glut Neurons | 21 | Up | 0.133 | 1.16E−02 | 0.879 | 1.061 |
| 22 | Sirt5 | Glut Neurons | 22 | Up | 0.166 | 1.30E−02 | 1.368 | 1.546 |
| 23 | Ccdc82 | Glut Neurons | 23 | Up | 0.161 | 1.30E−02 | 0.836 | 1.022 |
| 24 | Lrp8 | Glut Neurons | 24 | Up | 0.228 | 1.74E−02 | 1.230 | 1.582 |
| 25 | Pign | Glut Neurons | 25 | Up | 0.138 | 2.79E−02 | 1.154 | 1.258 |
| 26 | Nacc2 | Glut Neurons | 26 | Up | 0.153 | 2.89E−02 | 0.983 | 1.225 |
| 27 | Kcnq2 | Glut Neurons | 27 | Up | 0.157 | 3.55E−02 | 0.831 | 1.023 |
| 28 | Mef2a | Glut Neurons | 28 | Up | 0.132 | 4.15E−02 | 1.087 | 1.213 |
| 29 | Phf14 | Glut Neurons | 29 | Up | 0.110 | 4.40E−02 | 0.962 | 1.134 |
| 30 | Echs1 | Glut Neurons | 30 | Up | 0.113 | 4.69E−02 | 1.213 | 1.401 |
| 31 | Fnbp1 | Glut Neurons | 31 | Up | 0.181 | 5.02E−02 | 1.256 | 1.552 |
| 32 | Fyco1 | Glut Neurons | 32 | Up | 0.138 | 5.04E−02 | 1.016 | 1.249 |
| 33 | Akap5 | Glut Neurons | 33 | Down | −0.267 | 2.68E−07 | 4.186 | 4.098 |
| 34 | Lrrc17 | Glut Neurons | 34 | Down | −0.253 | 4.97E−07 | 3.433 | 3.362 |
| 35 | Calm1 | Glut Neurons | 35 | Down | −0.300 | 3.63E−06 | 2.413 | 2.058 |
| 36 | Ubb-ps | Glut Neurons | 36 | Down | −0.188 | 1.06E−04 | 1.073 | 0.796 |
| 37 | Snap25 | Glut Neurons | 37 | Down | −0.218 | 1.32E−04 | 1.630 | 1.375 |
| 38 | Atp6v0c | Glut Neurons | 38 | Down | −0.244 | 1.33E−04 | 1.847 | 1.533 |
| 39 | Ociad1 | Glut Neurons | 39 | Down | −0.295 | 1.79E−04 | 1.048 | 0.697 |
| 40 | Tecr | Glut Neurons | 40 | Down | −0.357 | 2.10E−04 | 1.710 | 1.295 |
| 41 | Hspa8 | Glut Neurons | 41 | Down | −0.298 | 3.39E−04 | 2.198 | 1.823 |
| 42 | Eef1a1 | Glut Neurons | 42 | Down | −0.235 | 4.10E−04 | 1.716 | 1.451 |
| 43 | Vps8 | Glut Neurons | 43 | Down | −0.194 | 5.54E−04 | 2.180 | 2.207 |
| 44 | Ubb | Glut Neurons | 44 | Down | −0.187 | 8.95E−04 | 1.879 | 1.610 |
| 45 | Hsp90ab1 | Glut Neurons | 45 | Down | −0.240 | 1.45E−03 | 2.228 | 1.883 |
| 46 | Hnrnpk | Glut Neurons | 46 | Down | −0.272 | 1.77E−03 | 1.013 | 0.696 |
| 47 | Fth1 | Glut Neurons | 47 | Down | −0.128 | 1.87E−03 | 1.889 | 1.785 |
| 48 | Ttc25 | Glut Neurons | 48 | Down | −0.137 | 2.22E−03 | 2.414 | 2.330 |
| 49 | Dlg1 | Glut Neurons | 49 | Down | −0.132 | 2.39E−03 | 2.642 | 2.560 |
| 50 | Rp138 | Glut Neurons | 50 | Down | −0.242 | 2.80E−03 | 1.299 | 1.051 |
| 51 | Xaf1 | Glut Neurons | 51 | Down | −0.129 | 3.69E−03 | 2.602 | 2.533 |
| 52 | Tuba1a | Glut Neurons | 52 | Down | −0.160 | 4.12E−03 | 1.535 | 1.310 |
| 53 | Atp1b1 | Glut Neurons | 53 | Down | −0.165 | 4.13E−03 | 1.263 | 1.044 |
| 54 | Eno1 | Glut Neurons | 54 | Down | −0.213 | 4.22E−03 | 1.230 | 0.996 |
| 55 | Mettl3 | Glut Neurons | 55 | Down | −0.124 | 4.27E−03 | 2.689 | 2.629 |
| 56 | Rab33b | Glut Neurons | 56 | Down | −0.142 | 4.34E−03 | 2.136 | 2.054 |
| 57 | Pola1 | Glut Neurons | 57 | Down | −0.165 | 4.53E−03 | 2.653 | 2.558 |
| 58 | Tuba1b | Glut Neurons | 58 | Down | −0.142 | 4.82E−03 | 1.116 | 0.911 |
| 59 | Snf8 | Glut Neurons | 59 | Down | −0.113 | 4.90E−03 | 2.777 | 2.719 |
| 60 | Ube2d3 | Glut Neurons | 60 | Down | −0.119 | 4.99E−03 | 2.641 | 2.578 |
| 61 | Hdac1 | Glut Neurons | 61 | Down | −0.144 | 5.25E−03 | 2.275 | 2.204 |
| 62 | Cox7c | Glut Neurons | 62 | Down | −0.213 | 5.36E−03 | 1.513 | 1.329 |
| 63 | Ywhae | Glut Neurons | 63 | Down | −0.195 | 5.91E−03 | 1.129 | 0.875 |
| 64 | Nme7 | Glut Neurons | 64 | Down | −0.206 | 5.91E−03 | 1.257 | 1.096 |
| 65 | Ndufa4 | Glut Neurons | 65 | Down | −0.256 | 5.97E−03 | 1.750 | 1.502 |
| 66 | Ctsl | Glut Neurons | 66 | Down | −0.161 | 5.99E−03 | 1.190 | 1.109 |
| 67 | Clic4 | Glut Neurons | 67 | Down | −0.122 | 6.14E−03 | 2.595 | 2.528 |
| 68 | Paox | Glut Neurons | 68 | Down | −0.147 | 6.73E−03 | 1.131 | 1.064 |
| 69 | Tmem69 | Glut Neurons | 69 | Down | −0.136 | 6.82E−03 | 2.031 | 1.951 |
| 70 | Mrp19 | Glut Neurons | 70 | Down | −0.115 | 7.11E−03 | 2.720 | 2.660 |
| 71 | Ttf1 | Glut Neurons | 71 | Down | −0.119 | 7.80E−03 | 3.436 | 3.381 |
| 72 | H2-B1 | Glut Neurons | 72 | Down | −0.136 | 7.80E−03 | 2.051 | 1.975 |
| 73 | Bcas2 | Glut Neurons | 73 | Down | −0.169 | 7.80E−03 | 2.263 | 2.198 |
| 74 | Actg1 | Glut Neurons | 74 | Down | −0.217 | 8.22E−03 | 1.616 | 1.339 |
| 75 | Parp1 | Glut Neurons | 75 | Down | −0.119 | 8.35E−03 | 2.504 | 2.434 |
| 76 | Cox6c | Glut Neurons | 76 | Down | −0.216 | 8.43E−03 | 1.591 | 1.368 |
| 77 | Zan | Glut Neurons | 77 | Down | −0.106 | 8.59E−03 | 2.853 | 2.811 |
| 78 | Nudt5 | Glut Neurons | 78 | Down | −0.113 | 8.78E−03 | 2.580 | 2.520 |
| 79 | Churc1 | Glut Neurons | 79 | Down | −0.118 | 9.05E−03 | 3.501 | 3.450 |
| 80 | Mrps26 | Glut Neurons | 80 | Down | −0.133 | 9.52E−03 | 2.088 | 2.013 |
| 81 | Capza2 | Glut Neurons | 81 | Down | −0.113 | 9.53E−03 | 2.848 | 2.785 |
| 82 | Dennd5b | Glut Neurons | 82 | Down | −0.131 | 1.01E−02 | 2.095 | 2.024 |
| 83 | Rpl23a | Glut Neurons | 83 | Down | −0.177 | 1.01E−02 | 1.044 | 0.877 |
| 84 | Atpaf2 | Glut Neurons | 84 | Down | −0.113 | 1.02E−02 | 2.606 | 2.545 |
| 85 | Eprs | Glut Neurons | 85 | Down | −0.132 | 1.07E−02 | 2.084 | 2.010 |
| 86 | Rps7 | Glut Neurons | 86 | Down | −0.231 | 1.07E−02 | 1.295 | 1.045 |
| 87 | Btbd7 | Glut Neurons | 87 | Down | −0.102 | 1.08E−02 | 2.741 | 2.687 |
| 88 | Calm2 | Glut Neurons | 88 | Down | −0.163 | 1.08E−02 | 2.158 | 1.921 |
| 89 | Cenpf | Glut Neurons | 89 | Down | −0.116 | 1.08E−02 | 2.493 | 2.429 |
| 90 | Slamf1 | Glut Neurons | 90 | Down | −0.104 | 1.11E−02 | 2.759 | 2.714 |
| 91 | Ccdc36 | Glut Neurons | 91 | Down | −0.136 | 1.11E−02 | 1.206 | 1.175 |
| 92 | Dnaja1 | Glut Neurons | 92 | Down | −0.233 | 1.22E−02 | 1.348 | 1.043 |
| 93 | Wdcp | Glut Neurons | 93 | Down | −0.104 | 1.22E−02 | 2.796 | 2.755 |
| 94 | Atg2a | Glut Neurons | 94 | Down | −0.134 | 1.26E−02 | 1.364 | 1.312 |
| 95 | Crebbp | Glut Neurons | 95 | Down | −0.110 | 1.27E−02 | 2.550 | 2.488 |
| 96 | Rpl6 | Glut Neurons | 96 | Down | −0.190 | 1.29E−02 | 1.037 | 0.808 |
| 97 | Sec23ip | Glut Neurons | 97 | Down | −0.107 | 1.34E−02 | 2.723 | 2.674 |
| 98 | Timeless | Glut Neurons | 98 | Down | −0.106 | 1.42E−02 | 2.635 | 2.582 |
| 99 | Foxr1 | Glut Neurons | 99 | Down | −0.113 | 1.45E−02 | 2.443 | 2.380 |
| 100 | Wdr12 | Glut Neurons | 100 | Down | −0.102 | 1.46E−02 | 2.816 | 2.770 |
| 101 | D3Ertd254e | Glut Neurons | 101 | Down | −0.179 | 1.49E−02 | 1.582 | 1.540 |
| 102 | Ccdc62 | Glut Neurons | 102 | Down | −0.131 | 1.55E−02 | 1.408 | 1.370 |
| 103 | Tmsb4x | Glut Neurons | 103 | Down | −0.167 | 1.58E−02 | 2.006 | 1.829 |
| 104 | Furin | Glut Neurons | 104 | Down | −0.132 | 1.67E−02 | 1.378 | 1.335 |
| 105 | Sec23b | Glut Neurons | 105 | Down | −0.109 | 1.72E−02 | 2.506 | 2.447 |
| 106 | Ppan | Glut Neurons | 106 | Down | −0.102 | 1.76E−02 | 2.725 | 2.680 |
| 107 | Cxcr2 | Glut Neurons | 107 | Down | −0.098 | 1.82E−02 | 2.760 | 2.721 |
| 108 | Atxn2 | Glut Neurons | 108 | Down | −0.131 | 1.83E−02 | 1.836 | 1.752 |
| 109 | Cep89 | Glut Neurons | 109 | Down | −0.104 | 1.93E−02 | 2.633 | 2.584 |
| 110 | Map4 | Glut Neurons | 110 | Down | −0.101 | 2.06E−02 | 2.841 | 2.794 |
| 111 | Pbld1 | Glut Neurons | 111 | Down | −0.106 | 2.10E−02 | 3.431 | 3.387 |
| 112 | Slc23a1 | Glut Neurons | 112 | Down | −0.114 | 2.10E−02 | 2.271 | 2.215 |
| 113 | Ndufc1 | Glut Neurons | 113 | Down | −0.150 | 2.15E−02 | 1.143 | 1.039 |
| 114 | Washc4 | Glut Neurons | 114 | Down | −0.092 | 2.19E−02 | 2.892 | 2.856 |
| 115 | Rps29 | Glut Neurons | 115 | Down | −0.136 | 2.20E−02 | 1.090 | 0.978 |
| 116 | Ube2k | Glut Neurons | 116 | Down | −0.099 | 2.20E−02 | 2.895 | 2.844 |
| 117 | Casc4 | Glut Neurons | 117 | Down | −0.109 | 2.21E−02 | 3.319 | 3.269 |
| 118 | Map1lc3b | Glut Neurons | 118 | Down | −0.132 | 2.21E−02 | 1.410 | 1.349 |
| 119 | Synb | Glut Neurons | 119 | Down | −0.118 | 2.25E−02 | 1.753 | 1.730 |
| 120 | P3h3 | Glut Neurons | 120 | Down | −0.111 | 2.28E−02 | 2.336 | 2.278 |
| 121 | Rtn1 | Glut Neurons | 121 | Down | −0.185 | 2.29E−02 | 1.456 | 1.210 |
| 122 | Atp5j | Glut Neurons | 122 | Down | −0.230 | 2.32E−02 | 1.433 | 1.199 |
| 123 | Loxl2 | Glut Neurons | 123 | Down | −0.098 | 2.45E−02 | 2.647 | 2.605 |
| 124 | Chpt1 | Glut Neurons | 124 | Down | −0.092 | 2.47E−02 | 2.804 | 2.768 |
| 125 | Aire | Glut Neurons | 125 | Down | −0.116 | 2.53E−02 | 2.229 | 2.157 |
| 126 | Kif3a | Glut Neurons | 126 | Down | −0.141 | 2.57E−02 | 1.279 | 1.198 |
| 127 | Zfp113 | Glut Neurons | 127 | Down | −0.090 | 2.59E−02 | 2.798 | 2.767 |
| 128 | Zcchc4 | Glut Neurons | 128 | Down | −0.113 | 2.63E−02 | 1.021 | 0.973 |
| 129 | Lsm8 | Glut Neurons | 129 | Down | −0.125 | 2.63E−02 | 1.158 | 1.062 |
| 130 | Atp5j2 | Glut Neurons | 130 | Down | −0.169 | 2.69E−02 | 1.017 | 0.851 |
| 131 | Chn1 | Glut Neurons | 131 | Down | −0.192 | 2.74E−02 | 1.491 | 1.219 |
| 132 | Fam193a | Glut Neurons | 132 | Down | −0.101 | 2.76E−02 | 2.523 | 2.472 |
| 133 | Enah | Glut Neurons | 133 | Down | −0.108 | 2.77E−02 | 2.253 | 2.212 |
| 134 | Trpc4ap | Glut Neurons | 134 | Down | −0.115 | 2.77E−02 | 1.759 | 1.729 |
| 135 | Atp5h | Glut Neurons | 135 | Down | −0.231 | 2.81E−02 | 1.628 | 1.341 |
| 136 | Atp5a1 | Glut Neurons | 136 | Down | −0.162 | 2.99E−02 | 1.038 | 0.832 |
| 137 | H2-T23 | Glut Neurons | 137 | Down | −0.173 | 3.05E−02 | 1.785 | 1.757 |
| 138 | Nedd4 | Glut Neurons | 138 | Down | −0.161 | 3.09E−02 | 1.461 | 1.358 |
| 139 | Fubp1 | Glut Neurons | 139 | Down | −0.093 | 3.12E−02 | 2.865 | 2.828 |
| 140 | Oaz1 | Glut Neurons | 140 | Down | −0.205 | 3.19E−02 | 1.407 | 1.200 |
| 141 | Snca | Glut Neurons | 141 | Down | −0.147 | 3.20E−02 | 1.008 | 0.835 |
| 142 | D6Wsu163e | Glut Neurons | 142 | Down | −0.101 | 3.31E−02 | 2.378 | 2.314 |
| 143 | Dhx30 | Glut Neurons | 143 | Down | −0.133 | 3.39E−02 | 1.377 | 1.310 |
| 144 | Nop58 | Glut Neurons | 144 | Down | −0.096 | 3.43E−02 | 2.688 | 2.645 |
| 145 | Ipo7 | Glut Neurons | 145 | Down | −0.119 | 3.44E−02 | 1.718 | 1.687 |
| 146 | Mtg2 | Glut Neurons | 146 | Down | −0.091 | 3.48E−02 | 2.672 | 2.635 |
| 147 | Gmeb1 | Glut Neurons | 147 | Down | −0.087 | 3.51E−02 | 2.807 | 2.782 |
| 148 | Polr2c | Glut Neurons | 148 | Down | −0.101 | 3.55E−02 | 2.468 | 2.412 |
| 149 | Dars | Glut Neurons | 149 | Down | −0.115 | 3.89E−02 | 1.426 | 1.380 |
| 150 | Cars2 | Glut Neurons | 150 | Down | −0.095 | 4.00E−02 | 2.558 | 2.514 |
| 151 | Cox6b1 | Glut Neurons | 151 | Down | −0.161 | 4.05E−02 | 1.229 | 1.063 |
| 152 | Hook3 | Glut Neurons | 152 | Down | −0.089 | 4.09E−02 | 2.588 | 2.556 |
| 153 | Aarsd1 | Glut Neurons | 153 | Down | −0.119 | 4.22E−02 | 1.760 | 1.682 |
| 154 | Matr3 | Glut Neurons | 154 | Down | −0.160 | 4.30E−02 | 1.827 | 1.685 |
| 155 | Rpl41 | Glut Neurons | 155 | Down | −0.192 | 4.30E−02 | 2.388 | 2.239 |
| 156 | Usp34 | Glut Neurons | 156 | Down | −0.099 | 4.38E−02 | 2.195 | 2.142 |
| 157 | Slc25a4 | Glut Neurons | 157 | Down | −0.125 | 4.39E−02 | 1.047 | 0.872 |
| 158 | Swt1 | Glut Neurons | 158 | Down | −0.085 | 4.48E−02 | 2.652 | 2.617 |
| 159 | Eif4a2 | Glut Neurons | 159 | Down | −0.234 | 4.53E−02 | 1.803 | 1.524 |
| 160 | Rhpn2 | Glut Neurons | 160 | Down | −0.114 | 4.55E−02 | 1.625 | 1.601 |
| 161 | Cpe | Glut Neurons | 161 | Down | −0.123 | 4.66E−02 | 1.199 | 1.060 |
| 162 | Exosc5 | Glut Neurons | 162 | Down | −0.109 | 4.66E−02 | 1.237 | 1.196 |
| 163 | Mmachc | Glut Neurons | 163 | Down | −0.102 | 4.72E−02 | 2.937 | 2.750 |
| 164 | Yae1d1 | Glut Neurons | 164 | Down | −0.143 | 4.73E−02 | 1.769 | 1.738 |
| 165 | Gclm | Glut Neurons | 165 | Down | −0.113 | 4.90E−02 | 1.127 | 0.984 |
| 166 | Eef1akmt3 | Astrocytes | 1 | Up | 0.336 | 4.52E−18 | 1.594 | 1.918 |
| 167 | Ilf2 | Astrocytes | 2 | Up | 0.295 | 3.20E−12 | 1.083 | 1.350 |
| 168 | Saa3 | Astrocytes | 3 | Up | 0.254 | 1.17E−09 | 1.487 | 1.697 |
| 169 | mt-Nd2 | Astrocytes | 4 | Up | 0.132 | 9.34E−03 | 1.273 | 1.340 |
| 170 | mt-Nd4 | Astrocytes | 5 | Up | 0.125 | 1.62E−02 | 2.105 | 2.165 |
| 171 | mt-Cytb | Astrocytes | 6 | Up | 0.093 | 2.64E−02 | 2.711 | 2.746 |
| 172 | mt-Atp6 | Astrocytes | 7 | Up | 0.106 | 3.09E−02 | 2.199 | 2.236 |
| 173 | Syne1 | Astrocytes | 8 | Up | 0.128 | 4.98E−02 | 0.956 | 1.068 |
| 174 | Akap5 | Astrocytes | 9 | Down | −0.219 | 8.63E−09 | 4.212 | 4.005 |
| 175 | Lrrc17 | Astrocytes | 10 | Down | −0.192 | 4.48E−08 | 3.471 | 3.291 |
| 176 | Pan3 | Astrocytes | 11 | Down | −0.179 | 5.23E−04 | 1.354 | 1.194 |
| 177 | Gapdh-ps15 | Astrocytes | 12 | Down | −0.157 | 2.50E−03 | 1.507 | 1.333 |
| 178 | Ythdc1 | Astrocytes | 13 | Down | −0.144 | 2.52E−03 | 1.933 | 1.822 |
| 179 | Tuba1a | Astrocytes | 14 | Down | −0.162 | 3.30E−03 | 1.651 | 1.483 |
| 180 | Ppp1r16b | Astrocytes | 15 | Down | −0.227 | 3.31E−03 | 2.730 | 2.539 |
| 181 | Unc13d | Astrocytes | 16 | Down | −0.146 | 5.25E−03 | 1.592 | 1.469 |
| 182 | Poldip2 | Astrocytes | 17 | Down | −0.142 | 8.30E−03 | 1.377 | 1.255 |
| 183 | Tpd52 | Astrocytes | 18 | Down | −0.145 | 8.95E−03 | 1.325 | 1.199 |
| 184 | Golga3 | Astrocytes | 19 | Down | −0.136 | 9.50E−03 | 1.622 | 1.515 |
| 185 | Atp6v0e2 | Astrocytes | 20 | Down | −0.138 | 1.22E−02 | 1.641 | 1.529 |
| 186 | Elp6 | Astrocytes | 21 | Down | −0.124 | 1.37E−02 | 1.819 | 1.701 |
| 187 | Tecr | Astrocytes | 22 | Down | −0.242 | 1.60E−02 | 1.825 | 1.562 |
| 188 | Nop58 | Astrocytes | 23 | Down | −0.094 | 1.62E−02 | 2.879 | 2.828 |
| 189 | Vps8 | Astrocytes | 24 | Down | −0.120 | 1.79E−02 | 2.185 | 2.087 |
| 190 | Capza2 | Astrocytes | 25 | Down | −0.082 | 1.99E−02 | 3.040 | 3.001 |
| 191 | Lztr1 | Astrocytes | 26 | Down | −0.130 | 1.99E−02 | 1.667 | 1.556 |
| 192 | Rpap1 | Astrocytes | 27 | Down | −0.125 | 2.41E−02 | 1.365 | 1.265 |
| 193 | Slc23a1 | Astrocytes | 28 | Down | −0.099 | 2.49E−02 | 2.484 | 2.421 |
| 194 | Mterf3 | Astrocytes | 29 | Down | −0.125 | 2.49E−02 | 1.393 | 1.289 |
| 195 | Fam193a | Astrocytes | 30 | Down | −0.088 | 2.57E−02 | 2.729 | 2.684 |
| 196 | Rfc2 | Astrocytes | 31 | Down | −0.121 | 2.57E−02 | 1.031 | 0.929 |
| 197 | Sumf2 | Astrocytes | 32 | Down | −0.127 | 2.67E−02 | 1.628 | 1.532 |
| 198 | Sec23ip | Astrocytes | 33 | Down | −0.086 | 2.73E−02 | 2.925 | 2.886 |
| 199 | Cars2 | Astrocytes | 34 | Down | −0.090 | 2.76E−02 | 2.749 | 2.703 |
| 200 | Aktip | Astrocytes | 35 | Down | −0.119 | 2.90E−02 | 1.751 | 1.656 |
| 201 | Aire | Astrocytes | 36 | Down | −0.106 | 2.91E−02 | 2.425 | 2.361 |
| 202 | Tubb2a | Astrocytes | 37 | Down | −0.113 | 3.17E−02 | 1.082 | 0.960 |
| 203 | Tuba1b | Astrocytes | 38 | Down | −0.115 | 3.20E−02 | 1.185 | 1.065 |
| 204 | Polr2c | Astrocytes | 39 | Down | −0.091 | 3.46E−02 | 2.662 | 2.613 |
| 205 | P3h3 | Astrocytes | 40 | Down | −0.092 | 3.46E−02 | 2.548 | 2.495 |
| 206 | Zcchc4 | Astrocytes | 41 | Down | −0.112 | 3.50E−02 | 1.107 | 0.999 |
| 207 | Calm1 | Astrocytes | 42 | Down | −0.155 | 3.59E−02 | 2.191 | 2.021 |
| 208 | Gje1 | Astrocytes | 43 | Down | −0.108 | 3.61E−02 | 1.051 | 0.961 |
| 209 | Haus8 | Astrocytes | 44 | Down | −0.116 | 3.67E−02 | 1.366 | 1.269 |
| 210 | Cox7a2 | Astrocytes | 45 | Down | −0.144 | 3.74E−02 | 1.186 | 1.052 |
| 211 | Calm2 | Astrocytes | 46 | Down | −0.130 | 3.93E−02 | 1.796 | 1.730 |
| 212 | Snf8 | Astrocytes | 47 | Down | −0.074 | 3.99E−02 | 2.974 | 2.945 |
| 213 | Hadhb | Astrocytes | 48 | Down | −0.103 | 4.04E−02 | 2.027 | 1.956 |
| 214 | Mrp19 | Astrocytes | 49 | Down | −0.074 | 4.30E−02 | 2.931 | 2.904 |
| 215 | Gmeb1 | Astrocytes | 50 | Down | −0.074 | 4.31E−02 | 2.991 | 2.962 |
| 216 | Ppan | Astrocytes | 51 | Down | −0.074 | 4.71E−02 | 2.945 | 2.916 |
| 217 | Parp1 | Astrocytes | 52 | Down | −0.079 | 4.71E−02 | 2.711 | 2.675 |
| 218 | Cep89 | Astrocytes | 53 | Down | −0.075 | 4.75E−02 | 2.853 | 2.822 |
| 219 | Mettl3 | Astrocytes | 54 | Down | −0.077 | 4.87E−02 | 2.874 | 2.842 |
| 220 | Uba2 | Astrocytes | 55 | Down | −0.113 | 4.89E−02 | 1.509 | 1.413 |
| 221 | Atg2a | Astrocytes | 56 | Down | −0.105 | 4.99E−02 | 1.485 | 1.418 |
| 222 | Mfsd4a | Oligodendrocytes | 1 | Up | 0.642 | 1.64E−24 | 0.457 | 1.058 |
| 223 | Eef1akmt3 | Oligodendrocytes | 2 | Up | 0.601 | 3.51E−24 | 1.486 | 2.089 |
| 224 | Saa3 | Oligodendrocytes | 3 | Up | 0.574 | 1.87E−21 | 1.341 | 1.909 |
| 225 | Ilf2 | Oligodendrocytes | 4 | Up | 0.463 | 2.34E−11 | 1.059 | 1.493 |
| 226 | Map3k20 | Oligodendrocytes | 5 | Up | 0.245 | 2.56E−05 | 2.111 | 2.351 |
| 227 | Upf3b | Oligodendrocytes | 6 | Up | 0.166 | 1.51E−04 | 3.053 | 3.197 |
| 228 | Jpt2 | Oligodendrocytes | 7 | Up | 0.265 | 3.14E−04 | 1.231 | 1.507 |
| 229 | Mmachc | Oligodendrocytes | 8 | Up | 0.154 | 6.55E−04 | 3.155 | 3.286 |
| 230 | Snhg11 | Oligodendrocytes | 9 | Up | 0.164 | 2.35E−03 | 2.755 | 2.917 |
| 231 | Sec14l1 | Oligodendrocytes | 10 | Up | 0.238 | 2.40E−03 | 1.047 | 1.281 |
| 232 | Them4 | Oligodendrocytes | 11 | Up | 0.233 | 3.16E−03 | 1.279 | 1.517 |
| 233 | Tulp1 | Oligodendrocytes | 12 | Up | 0.207 | 7.28E−03 | 1.675 | 1.893 |
| 234 | Pias4 | Oligodendrocytes | 13 | Up | 0.209 | 9.21E−03 | 1.422 | 1.649 |
| 235 | Ppp1r7 | Oligodendrocytes | 14 | Up | 0.231 | 9.22E−03 | 0.832 | 1.026 |
| 236 | Supt7l | Oligodendrocytes | 15 | Up | 0.169 | 9.50E−03 | 2.320 | 2.509 |
| 237 | Zfp827 | Oligodendrocytes | 16 | Up | 0.208 | 1.18E−02 | 1.058 | 1.284 |
| 238 | Zfp229 | Oligodendrocytes | 17 | Up | 0.199 | 1.52E−02 | 0.893 | 1.093 |
| 239 | Xcr1 | Oligodendrocytes | 18 | Up | 0.197 | 1.76E−02 | 0.878 | 1.078 |
| 240 | Psmb11 | Oligodendrocytes | 19 | Up | 0.191 | 2.39E−02 | 0.970 | 1.161 |
| 241 | Gsn | Oligodendrocytes | 20 | Up | 0.254 | 4.11E−02 | 0.807 | 1.005 |
| 242 | Slc10a7 | Oligodendrocytes | 21 | Up | 0.192 | 4.95E−02 | 1.329 | 1.538 |
| 243 | Lrrc17 | Oligodendrocytes | 22 | Down | −0.365 | 8.13E−10 | 3.122 | 2.805 |
| 244 | Akap5 | Oligodendrocytes | 23 | Down | −0.393 | 8.13E−10 | 3.865 | 3.517 |
| 245 | Vps8 | Oligodendrocytes | 24 | Down | −0.217 | 2.72E−02 | 1.860 | 1.703 |
| 246 | Tmem1311 | OPCs | 1 | Up | 0.585 | 2.87E−06 | 0.274 | 0.804 |
| 247 | Eef1akmt3 | OPCs | 2 | Up | 0.527 | 3.79E−05 | 1.333 | 1.870 |
| 248 | Mfsd4a | OPCs | 3 | Up | 0.460 | 3.00E−03 | 0.507 | 0.916 |
| 249 | Saa3 | OPCs | 4 | Up | 0.404 | 2.91E−02 | 1.206 | 1.654 |
| 250 | Ilf2 | OPCs | 5 | Up | 0.398 | 3.37E−02 | 0.909 | 1.310 |
| 251 | Akap5 | OPCs | 6 | Down | −0.320 | 1.33E−02 | 4.228 | 3.963 |
| 252 | Tmem181b-ps | OPCs | 7 | Down | −0.195 | 3.37E−02 | 0.210 | 0.055 |
| 253 | Lrrc17 | OPCs | 8 | Down | −0.286 | 3.37E−02 | 3.455 | 3.227 |
| 254 | Tmprss11a | OPCs | 9 | Down | −0.121 | 5.07E−02 | 0.127 | 0.015 |
| 255 | Eef1akmt3 | Microglia | 1 | Up | 0.394 | 4.98E−02 | 1.300 | 1.718 |
| 256 | Eef1akmt3 | Endothelial | 1 | Up | 0.511 | 1.06E−05 | 1.242 | 1.735 |
| 257 | Ilf2 | Endothelial | 2 | Up | 0.361 | 2.90E−02 | 0.830 | 1.206 |
| 258 | Saa3 | Endothelial | 3 | Up | 0.366 | 2.98E−02 | 1.061 | 1.407 |
| 259 | Slco1a4 | Endothelial | 4 | Up | 0.415 | 4.88E−02 | 0.851 | 1.231 |
| 260 | Akap5 | Endothelial | 5 | Down | −0.447 | 4.86E−05 | 4.085 | 3.573 |
| 261 | Lrrc17 | Endothelial | 6 | Down | −0.407 | 2.45E−04 | 3.330 | 2.859 |
| 262 | Vps8 | Endothelial | 7 | Down | −0.398 | 3.71E−03 | 2.125 | 1.667 |
| 263 | Flna | Vascular Cells | 1 | Up | 2.044 | 9.59E−07 | 0.364 | 2.309 |

TABLE 3
Intra-cell type analysis

| Cell Types | Overlap | Total Genes | Gene Names |
|---|---|---|---|
| Astrocytes, Endothelial, Glut | 6 | 1 | Eef1akmt3 |
| Neurons, Microglia, OPCs, | | | |
| Oligodendrocytes | | | |
| Astrocytes, Endothelial, Glut | 5 | 4 | Saa3 Lrrc17 Ilf2 Akap5 |
| Neurons, OPCs, | | | |
| Oligodendrocytes | | | |
| Astrocytes, Endothelial, Glut | 4 | 1 | Vps8 |
| Neurons, Oligodendrocytes | | | |
| Astrocytes, Glut Neurons | 2 | 23 | Mettl3 Zcchc4 Snf8 Tuba1a Gmeb1 Parp1 Tecr Cars2 Mrpl9 Cep89 |
| | | | Polr2c Fam193a Tuba1b Calm1 Calm2 Aire P3h3 Atg2a Ppan Capza2 |
| | | | Slc23a1 Nop58 Sec23ip |
| Glut Neurons, Oligodendrocytes | 2 | 1 | Mmachc |
| OPCs, Oligodendrocytes | 2 | 1 | Mfsd4a |
| Glut Neurons | 1 | 135 | Nacc2 Cox7c Eif4a2 Trpc4ap Clasp1 Dennd5b Fth1 Zfp488 Atp5h |
| | | | Fgd4 Xaf1 Btbd7 Ttf1 Ccdc82 Ubb-ps Washc4 Sirt5 Chn1 Atxn2 |
| | | | Tmsb4x Paox Ndufa4 Echs1 Dhx16 Phf14 Atp5j Rpl23a Casc4 Rpl6 |
| | | | Ube2d3 Clic4 Hspa8 Wdr12 Churc1 Eprs Matr3 Oaz1 Map4 Hsp90ab1 |
| | | | D10Wsu102e H2-T23 Mtg2 Nudt5 Mef2a Chpt1 Ndufc1 Mrps26 |
| | | | Snap25 Cox6c Actg1 Lsm8 Cyb5r4 Rps7 Nav2 Eef1a1 Slc25a4 Foxr1 |
| | | | Pola1 D3Ertd254e Wdcp Ociad1 Acad9 Cpe Ubb Fut8 Swt1 Hsd3b2 |
| | | | Gclm Dlg1 Hook3 Pbld1 Mdga2 Dars Fnbp1 Sec23b Enah Lrp8 Il17ra |
| | | | Atpaf2 Nme7 Furin Kif3a Nedd4 Timeless Ctsl Zfp113 Hdac1 Rpl41 |
| | | | Aarsd1 Rtn1 Usp34 D6Wsu163e Zan Tmem69 Kcnj16 Ttc25 Map1lc3b |
| | | | Atp5j2 Atp5a1 Bcas2 Nvl Snca Zfp369 Crebbp Ipo7 H2-Bl Kcnq2 |
| | | | Yae1d1 Rpl38 Cenpf Synb Atp1b1 Atp6v0c Slamf1 Rab33b E2f1 |
| | | | Exosc5 Ccdc62 Dnaja1 Fyco1 Rhpn2 Hnrnpk Fubp1 Ube2k Ccdc36 |
| | | | Eno1 Pign Cxcr2 Dhx30 Mrpl48 Ywhae Dpp10 Cox6b1 Rps29 Loxl2 |
| Astrocytes | 1 | 27 | Golga3 Elp6 Hadhb mt-Nd4 Tpd52 mt-Atp6 mt-Nd2 Poldip2 Ppp1r16b |
| | | | Rfc2 Aktip Lztr1 Mterf3 Uba2 Rpap1 Gapdh-ps15 Cox7a2 Atp6v0e2 |
| | | | Gje1 mt-Cytb Pan3 Unc13d Ythdc1 Syne1 Haus8 Sumf2 Tubb2a |
| Oligodendrocytes | 1 | 16 | Supt7l Map3k20 Zfp827 Zfp229 Psmb11 Sec14l1 Upf3b Them4 |
| | | | Snhg11 Ppp1r7 Slc10a7 Tulp1 Jpt2 Pias4 Xcr1 Gsn |
| OPCs | 1 | 3 | Tmprss11a Tmem181b-ps Tmem131l |
| Endothelial | 1 | 1 | Slco1a4 |
| Vascular | 1 | 1 | Flna |

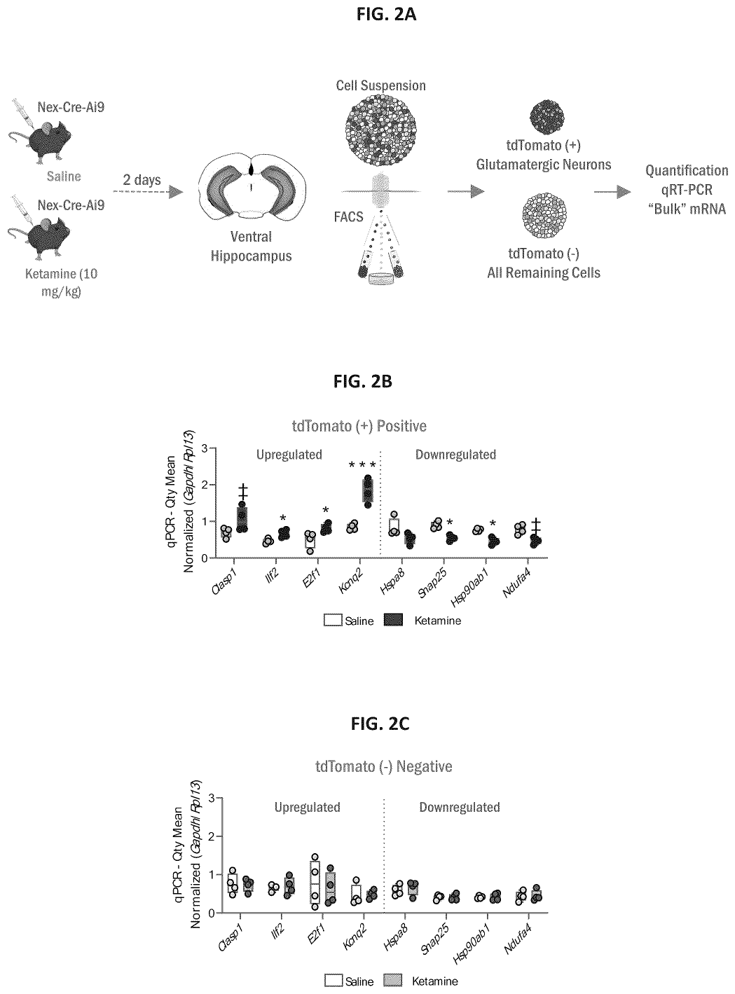

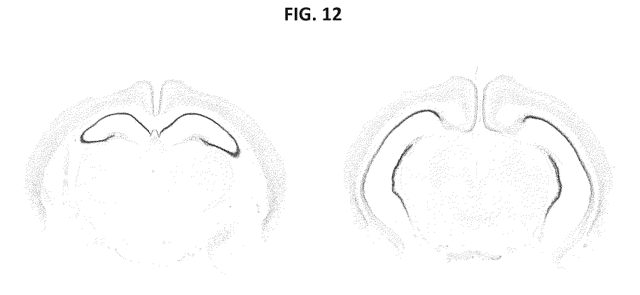

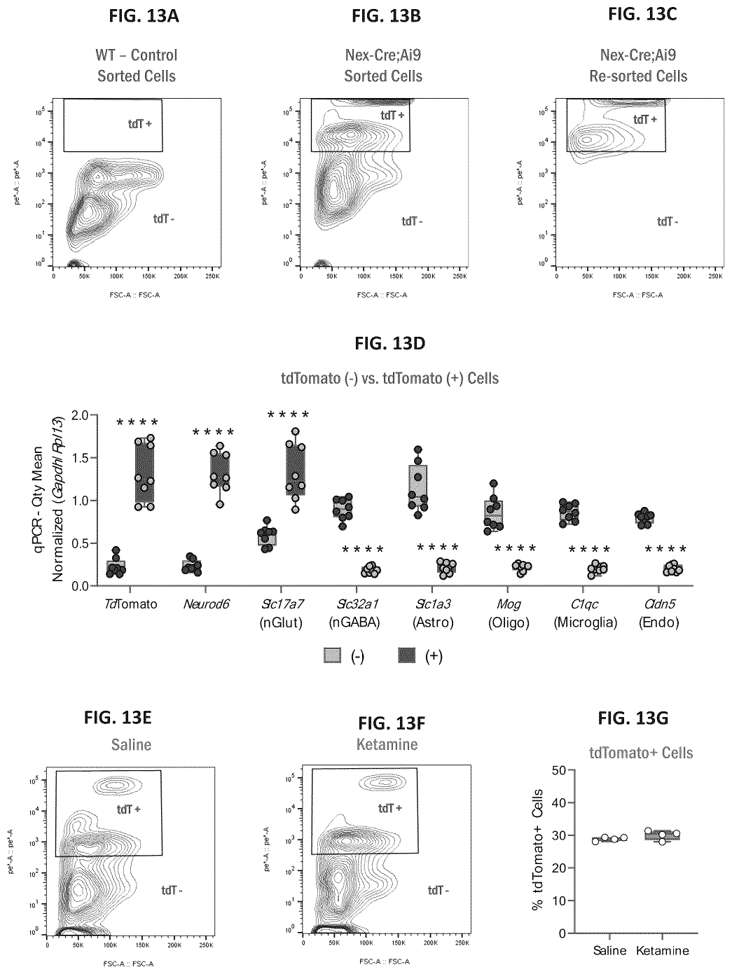

The glutamatergic neurons were the most interesting cell type based on their multi-genic response (165 DEGs), as well as their known roles modulating the antidepressant effects of ketamine (FIG. 1D). In order to further investigate these findings, a conditional reporter mouse line (Nex-Cre-Ai9) was generated where most glutamatergic neurons of the forebrain, including the hippocampus, are fluorescently labeled by tdTomato (Ai9) except for neurons in the dentate gyrus where Neurod6, the promoter used to target glutamatergic neurons driving Cre expression, is not expressed (FIG. 12) [J. Hartmann et al., Mol Psychiatry (2017) 22: 466-475]. Following the same paradigm used in the original cohort, mice were group-housed and injected with (R,S)-ketamine (10 mg/kg BW) or a saline vehicle control. The vHipp of these mice was dissected 36 hours later (i.e. 2 days). Single-cell suspensions were prepared and individual cells were sorted using fluorescence activated cell sorting (FACS) (FIG. 2A) into two separate pools of cells from each mouse. One pool contained glutamatergic neurons (Ai9+) and a second contained all remaining cell types of the vHipp (Ai9-) (FIG. 2A and FIGS. 13A-C). To confirm the presence of glutamatergic neurons in the Ai9+pool, mRNA was quantified and higher levels of the genes coding for tdTomato, the fluorophore used to label the cells, Neurod6, the promoter (−Cre) used to target glutamatergic neurons, as well as Slc17a7, a known marker of glutamatergic neurons, were found as compared to cells from the Ai9—pool (FIG. 13D). In addition, mRNA expression levels of established cell-type-specific markers for other cell types in the brain, such as Slc32a1 (GABA neurons), Slc1a3 (astrocytes), Mog (oligodendrocytes), C1qc (microglia), and Cldn5 (endothelial cells), were quantified and it was uncovered that Ai9+ cells expressed lower levels of these genes, as compared to Ai9—cells (FIG. 13D). These results validated the method used and confirmed the presence of glutamatergic neurons in the Ai9+ cells. These two separate pools of cells were then used to validate the scRNA-seq findings in glutamatergic neurons at the population level using quantitative real-time polymerase chain reaction (qPCR). No significant differences in the total number of Ai9+sorted cells were found from the ketamine or saline groups, suggesting no major changes in cell composition following ketamine treatment and confirming the scRNA-seq results (FIGS. 13E-G). As a proof of principle, 8 of the top DEGs in glutamatergic neurons (4 up-regulated and 4 down-regulated) were selected from the scRNA-seq analysis (Table 2B, above). It was uncovered that most of the genes quantified in the Ai9+ cells were significantly dysregulated, and the direction was consistent with the scRNA-seq findings (FIG. 2B). Interestingly, among these genes, the voltage-gated potassium channel subfamily Q member 2 (Kcnq2), showed the strongest effect after ketamine treatment (p<0.001, FC=2.2). No significant differences were found in mRNA levels for any of the 8 DEGs in the Ai9—cells (FIG. 2C).

The mRNA expression of these 8 genes was also quantified using brain punches from the vHipp of a new cohort of ketamine—or saline-treated mice in order to compare whole tissue “bulk” versus cell-type-specific methods. Interestingly, the results showed small changes in a direction that was consistent with the scRNA-seq results, however, these effects did not reach statistical significance (FIGS. 14A-B). Overall, these results demonstrated that ketamine treatment elicits cell-type-specific gene expression changes in the vHipp of mice. Furthermore, these findings also suggest that these cell-type-specific molecular changes could be diluted or masked in alternative studies using bulk tissue analyses.

### Example 2

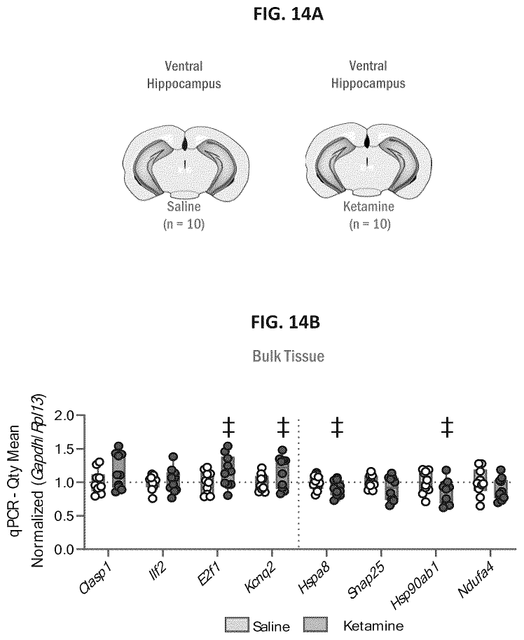

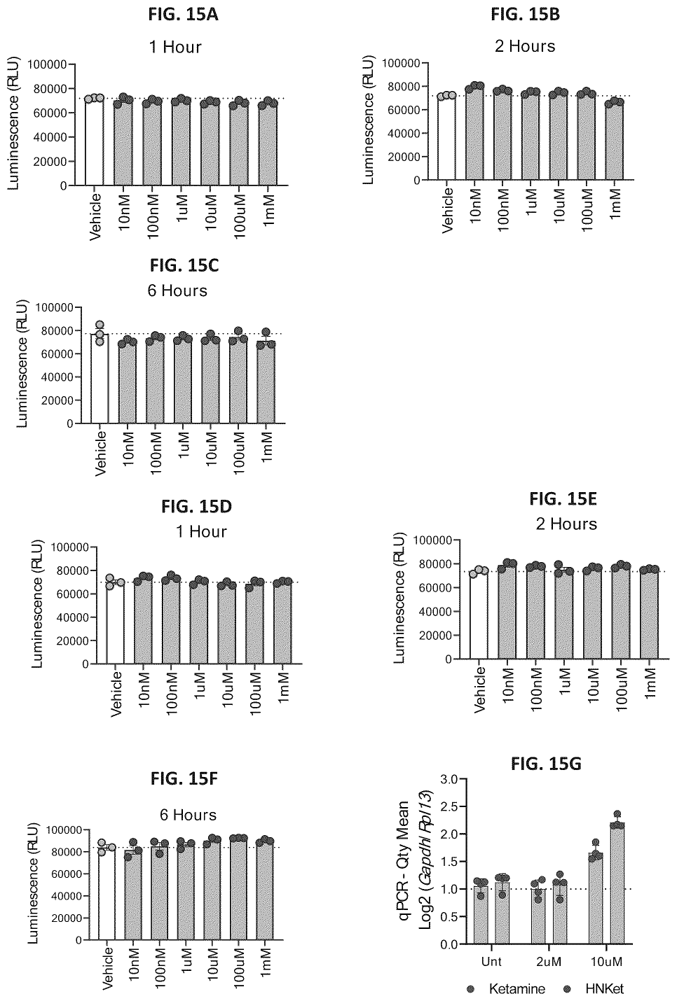

Ketamine treatment regulates Kcnq2 in primary hippocampal neurons To corroborate the previous findings in glutamatergic neurons, it was next examined whether treatment of primary hippocampal neurons with either (R,S)-ketamine or its active metabolite, (2R,6R)-hydroxynorketamine (HNKet), could modify the mRNA expression of the 8 genes tested earlier in glutamatergic neurons. Mouse primary hippocampal neurons are mostly made up of glutamatergic neurons and therefore make a very good model system to further validate the previous in vivo findings. Primary neurons were cultured for 21 days and then stimulated with ketamine (10 μM) or HNKet (10 μM) for 2, 12, 24, or 48 hours and compared to untreated and saline controls (FIGS. 3A-H, FIGS. 15A-G) and after review of the available literature, showing that at 10 μM both ketamine and HNKet can elicit relevant molecular and electrophysiological effects in vitro (Lazarevic et al., Mol Psychiatry 2021). No significant differences were found in the expression of any of the eight genes tested after treatment with a saline vehicle control, as compared to the untreated primary neurons (FIGS. 3A-H). However, significant changes were found after ketamine and HNKet treatment in the mRNA expression of Hspa8, Snap25, Hsp90abl, Ndufa4, IlJ2, E2f1, and Kcnq2. Notably, the mRNA changes seen in primary neurons, after ketamine or HKet treatment, were in a direction that was consistent with the scRNA-seq findings (FIGS. 3A-H). Among the DEGs, Kcnq2 displayed the largest changes in gene expression after treatment, showing a significant upregulation at all time points tested with both ketamine and HNKet. These findings, both in vivo and in vitro, suggested that Kcnq2 is transcriptionally regulated by ketamine and could be an important target of ketamine action in glutamatergic neurons of the hippocampus.

### Example 3

Ketamine Increases KCNQ Channel Currents in Hippocampal Neurons In Vitro and In Vivo

The Kcnq2 gene encodes for the Kv7.2 protein, a well characterized slow acting, voltage-gated potassium channel that plays a critical role in the regulation of neuronal excitability. It is known that Kv7.2 and the Kv7.3 protein (Kncq3 gene) can form KCNQ (Kv7) homo- or heterotetramers that can generate a signature M-current, which ultimately modulates the overall excitability of neurons in the central nervous system. To investigate the KCNQ channel as a mediator of ketamine action, mouse primary hippocampal neurons were treated with HNKet (10 μM) or a saline control for 24 hours and M-current density (I_{M}) was quantified using whole-cell voltage-clamp recordings (FIG. 3I). HNKet treatment was chosen for this experiment based in the previous findings showing a stronger effect of this compound over ketamine in primary neurons (FIGS. 3A-H). Consistent with the mRNA results, it was found that neurons treated with HNKet displayed a significant increase in I_{M} current density as compared to saline treated controls (FIGS. 3J-K), suggesting that HNKet increases the surface expression of KCNQ channels in primary hippocampal neurons after 24 hours of treatment.

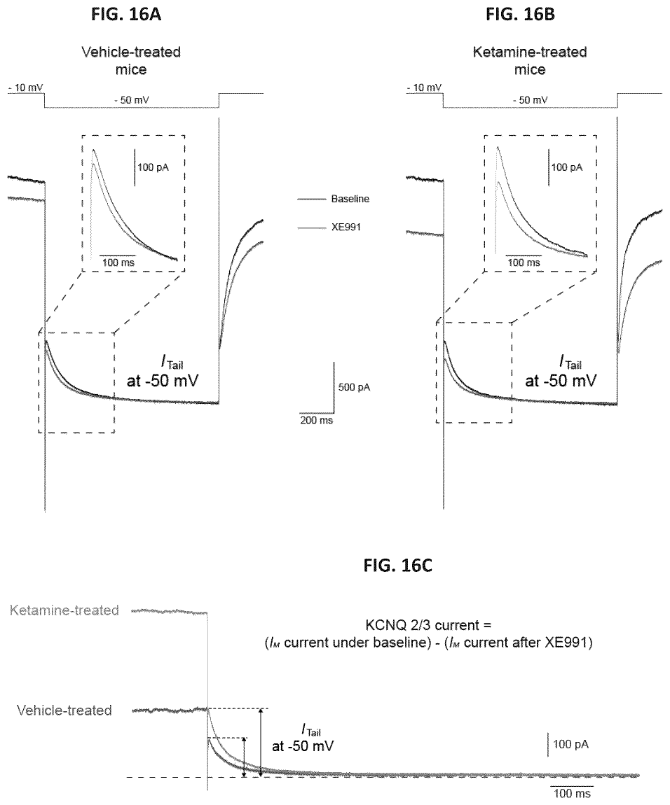

In an attempt to replicate these findings in vivo, ex vivo patch-clamp recordings were performed from acute hippocampal slices testing the effects of ketamine treatment on I_{M} current density (FIG. 3L). All mice were housed and treated as described in the original design (FIG. 1F). Briefly, mice were injected (i.p) with (R,S)-ketamine (10 mg/kg BW) or a saline control and sacrificed 36 hours later. Electrophysiological recordings were performed specifically from glutamatergic (CA1 pyramidal) neurons of the vHipp. Consistent with the results in vitro, it was found that ketamine-treated mice showed a significant increase in I_{M} current density as compared to saline-treated controls (FIGS. 3M-N, FIGS. 16A-C), suggesting that ketamine treatment also increases the expression of KCNQ channels in vivo. These findings indicated that the Kcnq2 mRNA expression increase observed in glutamatergic neurons after ketamine treatment is accompanied by a significant gain in the number of functional KCNQ channels expressed in glutamatergic neurons of the hippocampus both in vitro and in vivo. Together, these findings further supported the idea that ketamine regulates KCNQ expression and suggested that modulation of these channels in glutamatergic neurons of the vHipp could be a potential druggable target for the treatment of major depressive disorder (MDD).

### Example 4 shRNA knockdown of Kcnq2 in the vHipp reduces the antidepressant effects of ketamine

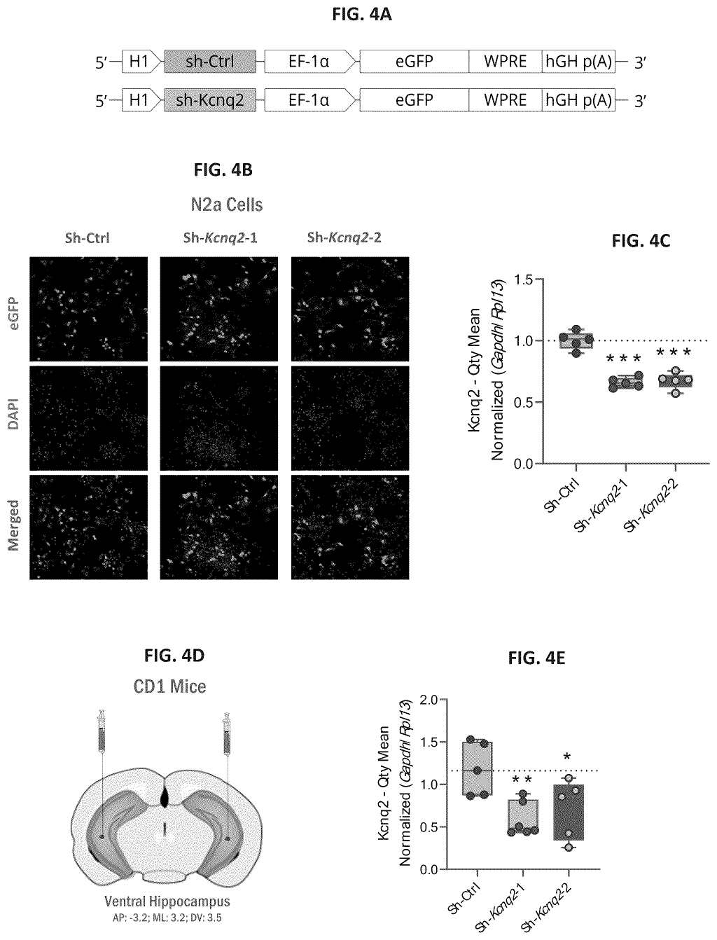

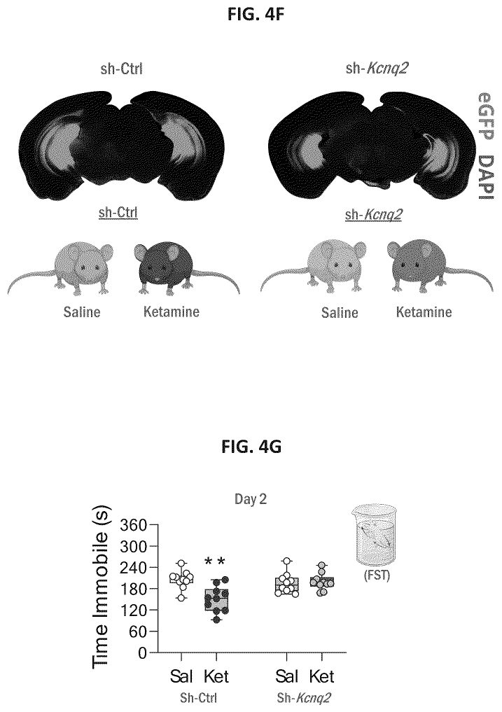

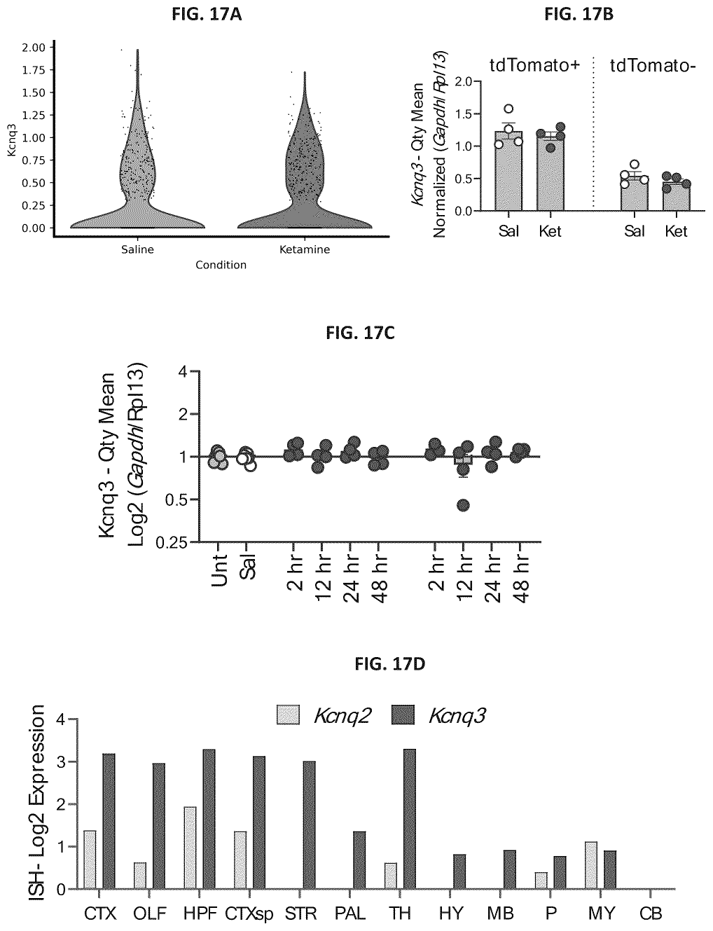

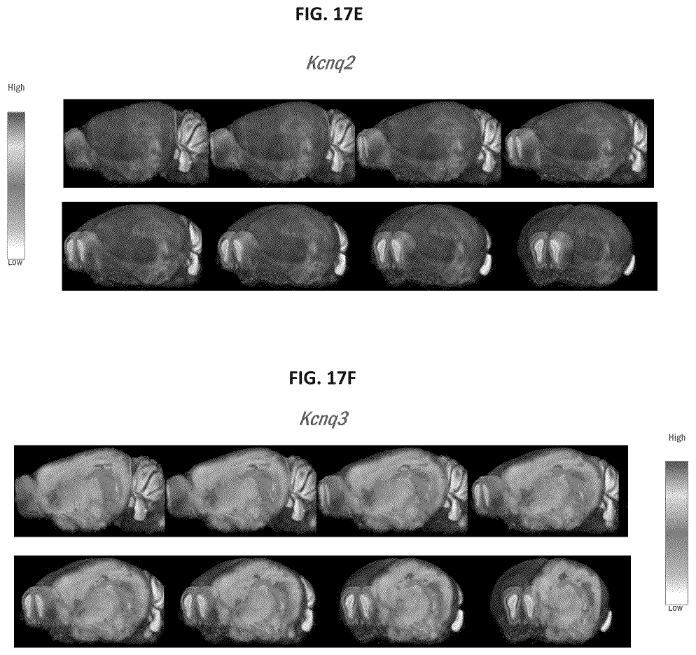

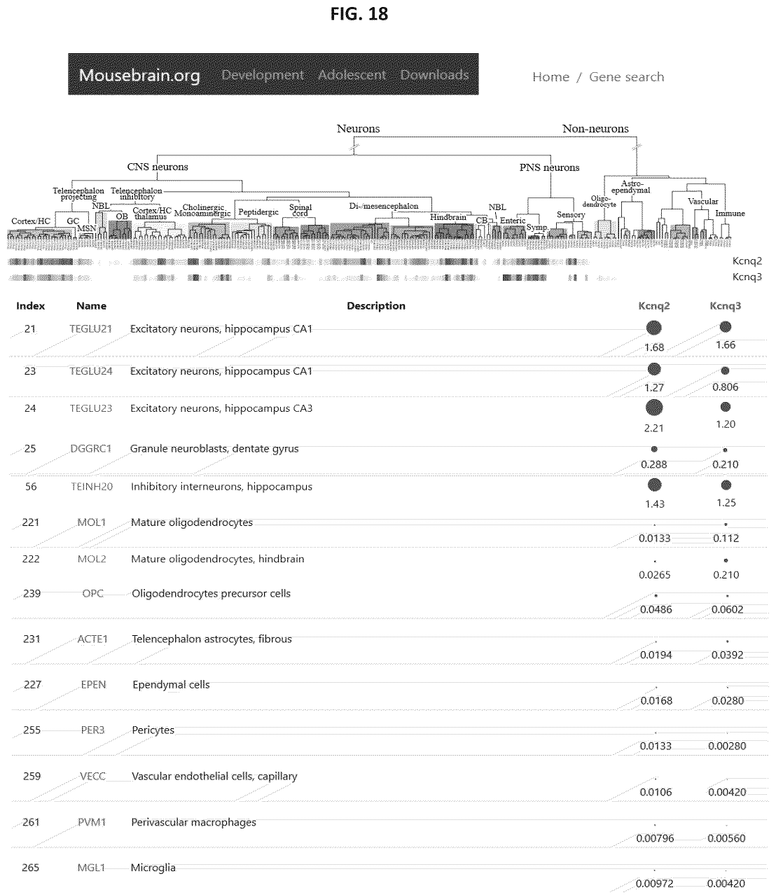

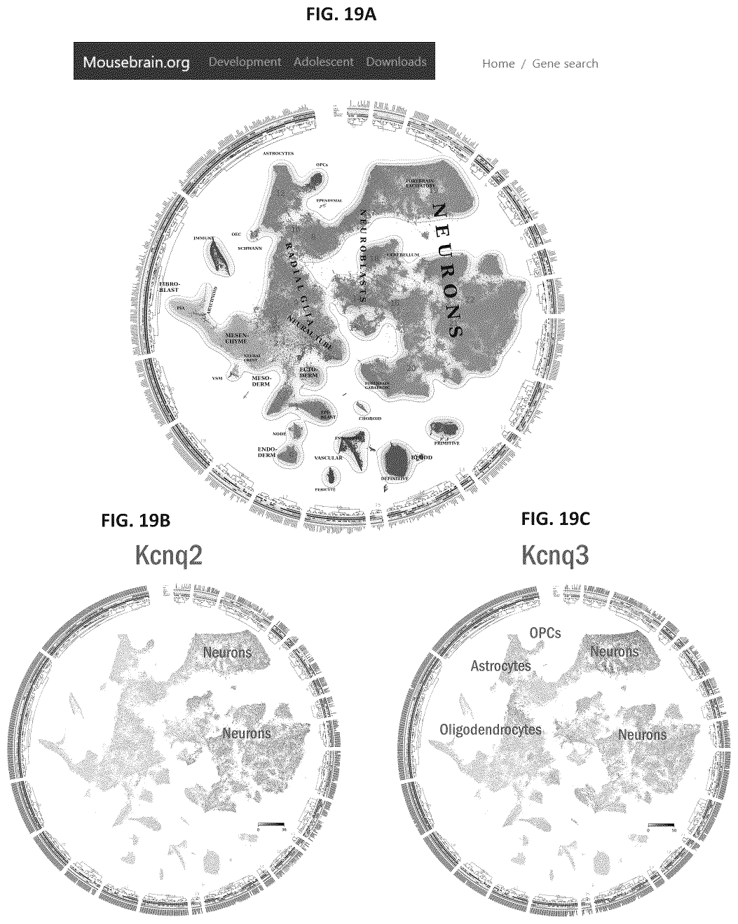

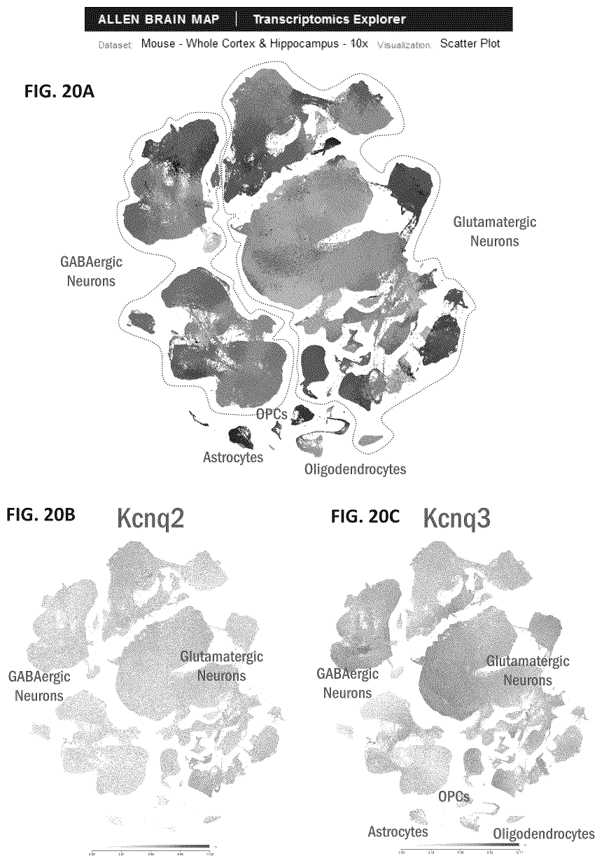

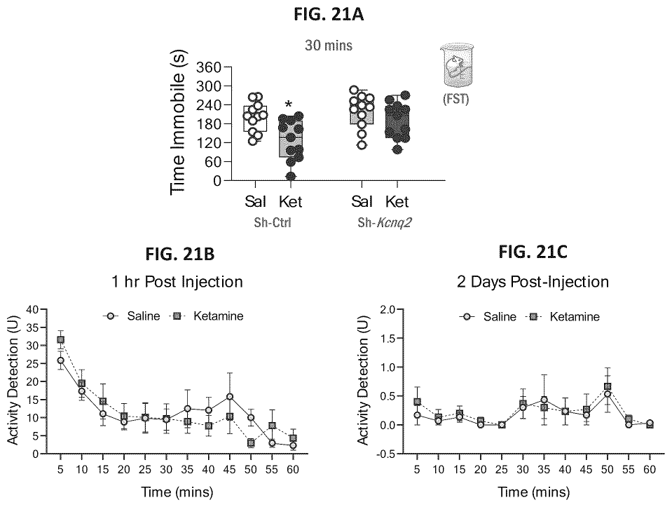

The M channel (KCNQ) is formed by the proteins encoded by the Kcnq2 and Kcnq3 genes, both integral membrane proteins. A significant upregulation of Kcnq2 was found in glutamatergic neurons after ketamine treatment in the original cohort (FIG. 1D and Table 3, above), in FACS-sorted sample (FIG. 2B), as well as in primary hippocampal neurons (FIGS. 3A-H). However, no significant differences were found in the mRNA expression of Kcnq3 after ketamine treatment in any of the experiments previously described (Table 2B, above, and FIGS. 17A-C), suggesting that ketamine produces an effect that is specific to Kcnq2, but not Kcnq3. In order to explore the expression of Kcnq2 and Kcnq3 in the brain, publicly available in situ hybridization (ISH) data from 12 different regions of the mouse brain were examined [E. S. Lein et al., Nature (2007) 445: 168-176]. It was uncovered that Kcnq3 is expressed higher than Kncq2 in almost all regions tested, except for the medulla (FIGS. 17D-F). In addition, Kcnq2 showed its highest expression in the hippocampus formation, while Kcnq3 was highly expressed throughout multiple brain regions including the olfactory areas, cortex, striatum, thalamus, and hippocampus, among others (FIGS. 17D-F). In addition, the single-cell data, as well as other publicly available single-cell data sets, show that Kcnq2 is exclusively expressed in neurons, while Kncq3 is expressed in neurons, astrocytes, oligodendrocytes and OPCs [J. P. Lopez et al., Sci Adv (2021) 7] (FIGS. 18, 19A-C and 20A-C). These findings reinforced the idea that Kcnq2 plays an important role in the hippocampus, thus positioning this brain region as a good candidate for in vivo viral manipulations. To further investigate and functionally explore the role of Kcnq2 in mediating the antidepressant-like effects of ketamine, viral AAV constructs were designed to knockdown Kcnq2 in vivo (FIG. 4A). Transfection of Neuro2a (N2a) cultured cells and viral injection into the vHipp of adult mice, resulted in a significant decrease of Kcnq2 mRNA levels, both in vitro and in vivo (FIGS. 4B-E). shRNA-Kcnq2 or shRNA-scramble control AAV viruses were bilaterally injected into the vHipp of a new group of mice. Four weeks after viral injection, half of the mice were randomly selected to receive an (R,S)-ketamine (10 mg/kg BW) or saline injection, and the fast-acting antidepressant effects of ketamine were assessed using the FST (FIG. 4F). In the group of mice treated with an shRNA-scramble control, a significant decrease in immobility time during the FST was found in mice treated with ketamine, as compared to saline-treated controls (FIGS. 21A and 4G, left). Notably, the antidepressant-like effects of ketamine were no longer detected in mice expressing the shRNA-Kcnq2 virus (FIGS. 21A and 4G, right). These results indicated that the vHipp is an important site for Kcnq2 function and the antidepressant effects of ketamine. Locomotor activity was also assessed to rule out any confounding effects of hyperlocomotion in the FST after ketamine treatment and found no differences in total activity between groups (FIGS. 21B-C). These results indicated that the vHipp is an important site for Kcnq2 function and the sustained antidepressant effects of ketamine.

### Example 5

Chronic Stress Exposure and Ketamine Treatment Modulate Kcnq2 mRNA in the Ventral Hippocampus

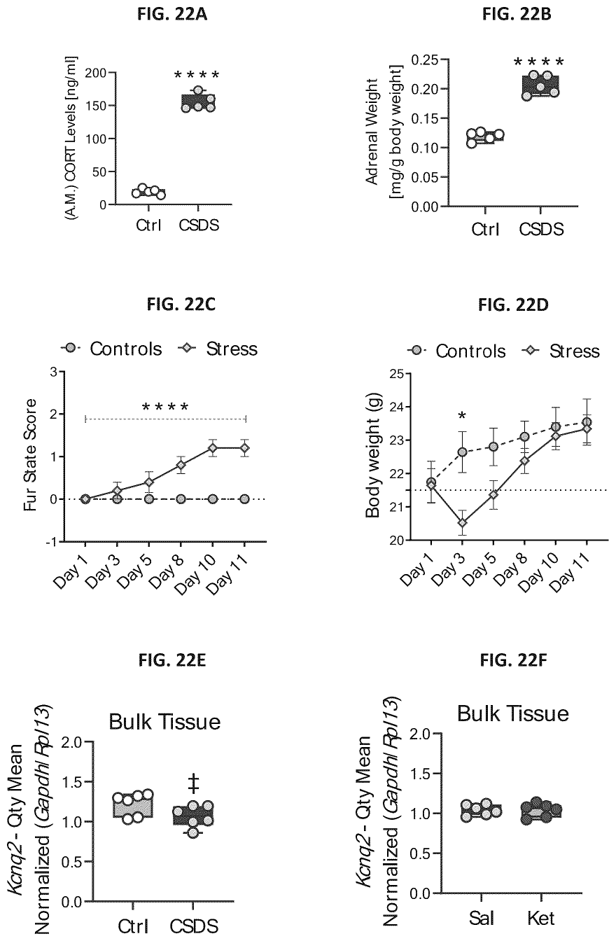

To further investigate the role of Kcnq2 in glutamatergic neurons of the vHipp, a new group of Nex-Cre-Ai9 mice were subjected to the chronic social defeat stress (CSDS) model, a validated and commonly used paradigm to induce long-lasting, depression- and anxiety-like endophenotypes in mice (FIG. 5A) [J. P. Lopez et al., Sci Adv (2021) 7]. Ten days of CSDS exposure resulted in hallmark features of chronically stressed mice, including a significant increase in basal corticosterone (CORT) levels, enhanced adrenal weight, and reduced fur quality (FIGS. 22A-D). In the FST, chronically stressed mice showed a significant increase in immobility time, as compared to non-stressed controls (FIG. 5B). End point and tissue collection were performed 24 hours after the last social defeat session (day 11). This was done to capture the cumulative effects of chronic stress, rather than the acute effects of the last defeat session. The vHipp of stressed and control mice were dissected and individual cells were sorted using FACS, as described in FIG. 2A. The results illustrate that Kcnq2 mRNA expression was decreased only in glutamatergic neurons (tdTomato+) of the vHipp, as no significant differences were found in the remaining cell-types (tdTomato-) (FIG. 5C). A significant difference in Kcnq2 mRNA expression between tdTomato positive and negative cells was also found, once again confirming that Kcnq2 is enriched in glutamatergic neurons of the vHipp. Finally, to access the bulk mRNA expression levels of Kcnq2 following chronic stress, its expression was quantified using brain punches from the vHipp of a new cohort of chronically stressed and control mice. Interestingly, these results showed a decrease in Kcnq2 mRNA levels in stressed mice, as compared to controls, however, this effect did not reach statistical significance (FIG. 22E, pva1=0.08).

In order to test whether CSDS-induced Kcnq2 decrease could be reversed by ketamine treatment, a new group of Nex-Cre-Ai9 mice was exposed to the CSDS model for 10 days. One day after the last social defeat (day 11), mice were treated with either (R,S)-ketamine (10 mg/kg/body weight) or a saline control (i.p) (FIG. 5D). The antidepressant effects of ketamine were assessed two days after treatment (day 13), using the FST. In addition, the vHipp of saline and ketamine-treated CSDS-mice were dissected and individual cells were sorted using FACS. Interestingly, it was uncovered that ketamine reversed the effects of chronic stress in the FST (FIG. 5E) and Kcnq2 mRNA in glutamatergic neurons (FIG. 5F). Specifically, ketamine treatment in CSDS-mice lead to a significant decrease in immobility time during the FST, as compared to saline-treated controls (FIG. 5E). Most importantly, Kcnq2 mRNA expression was significantly increased to baseline levels (as seen in non-stressed controls, FIG. 5C) in ketamine-treated CSDS-mice as compared to saline-treated controls (FIG. 5F). In addition, these effects were specific to glutamatergic neurons (tdTomato+) of the vHipp (FIG. 5F, left), since no significant differences were found in tdTomato negative cells (FIG. 5F, right). Specifically, no significant increase of Kcnq2 mRNA was found in CSDS-mice treated with ketamine using bulk mRNA (FIG. 22F). These results demonstrate that Kcnq2 mRNA is altered after chronic stress exposure in glutamatergic neurons of the vHipp and that these effects can be reversed by ketamine treatment. Furthermore, these results suggest that these cell-type-specific effects could be diluted or distorted in other studies using brain homogenates or whole tissue samples.

### Example 6

Ketamine Regulates Kcnq2 Via Ca²⁺ and Calmodulin/Calcineurin Signaling

Having identified Kncq2 as a potential target of ketamine, the inventors wanted to further investigate a plausible mechanism of how ketamine can transcriptionally upregulate Kcnq2 mRNA levels to exert its antidepressant-like effects in mice. Previous studies have shown that an increase in intracellular calcium (Ca²⁺) levels causes the activation of calmodulin (CaM), an ubiquitous calcium-sensor [D. Chin and A. R. Means, Trends Cell Biol (2000) 10: 322-328]. CaM then directly binds to the C-terminal domains of Kcnq2 which leads to a fast activation of KCNQ activity and regulation of neuronal excitability [X. Zhou et al., Am J Transl Res (2016) 8: 5610-5618]. Other studies have further described how Kcnq2 mRNA is regulated by the activation of calcineurin (CaN), a Ca²⁺ and CaM dependent serine/threonine protein phosphatase, as well as the transcription factor NFAT (Nuclear Factor of Activated T-Cell), via the A-kinase-anchoring protein 5 (AKAP5), also known as AKAP79/150 [J. Zhang and M. S. Shapiro, Neuron (2012) 76: 1133-1146]. In a series of complementary experiments, Zhang and Shapiro showed that an increase in Ca²⁺ signaling causes activation of CaM, which then binds to AKAP5 and CaN. The transcription factor NFAT is then dephosphorylated by the AKAP5/CaM/CaN complex, which leads to translocation of NFAT to the nucleus, where it acts particularly on Kcnq2 gene regulatory elements, as opposed to Kcnq3 [J. Zhang and M. S. Shapiro, (2012), supra]. Enhanced Kcnq2 transcription leads to increased M-channel expression and regulation of neuronal excitability. Interestingly, the CaM genes, Calm1 and Calm2, as well as Akap5 were among the DEGs of the scRNA-seq analysis (Table 2B, above). To selectively investigate if ketamine can regulate Kcnq2 via Ca²⁺, CaM, CaN or AKAP5 signaling, it was examined whether pharmacological inhibition of the key components of this pathway could interfere with the regulation of Kcnq2 mRNA by ketamine in primary mouse neurons. Primary hippocampal neurons were stimulated with either saline, (2R,6R)-HNKet, or a combination of (2R,6R)-HNKet plus nifedipine (L-type Ca²⁺ channel blocker), W-7 hydrochloride (CaM inhibitor), or cyclosporine-A (CaN inhibitor) for 30 minutes, 1, 2, or 6 hours, and compared to a group of untreated controls (FIGS. 6A-D). No significant differences were found in the expression of Kcnq2 after 30 minutes of treatment (FIG. 6A). However, consistent with the results shown in FIGS. 3A-H, a significant upregulation of Kcnq2 mRNA was found after 1, 2 and 6 hours of stimulation with HNKet (FIGS. 6B-D). Interestingly, blockade of L-type Ca²⁺ channels and CaM by nifedipine and W-7, respectively, eliminated the upregulation of Kcnq2 by ketamine at all time points tested (FIGS. 6B-D). On the other hand, inhibition of CaN by cyclosporine-A only abolished the effects of ketamine on Kcnq2 after 6 hours of treatment (FIG. 6D). These results, summarized in FIG. 6E, suggest that the ketamine-induced increase expression of Kcnq2 was blocked by nifedipine, W-7, or cyclosporine-A, suggesting a critical role for L-type Ca²⁺ channels, CaM and CaN for transcriptional regulation of Kcnq2 by ketamine treatment (e.g. at later time points).

### Example 7

Pharmacological Manipulation of KCNQ Channels Modulates Antidepressant-Like Behaviors in Mice

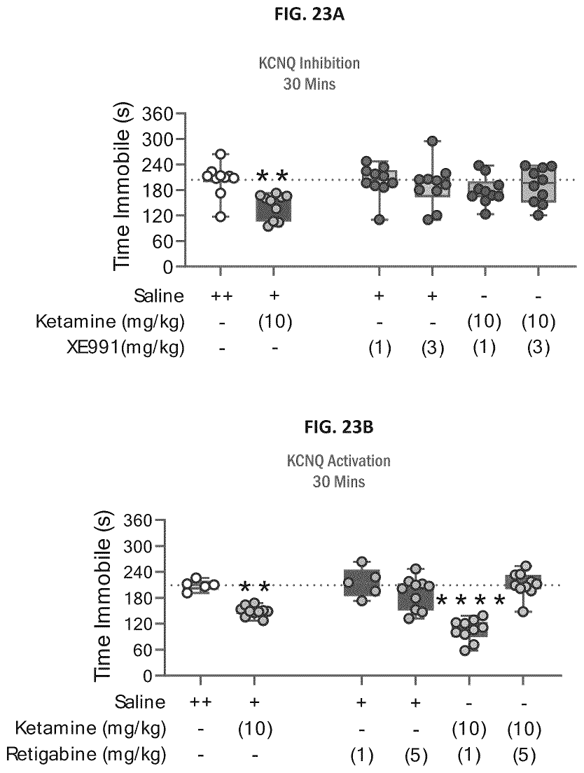

Previous studies in rodents have shown that KCNQ function can be regulated with highly-specific agonists and antagonists [V. Barrese et al., Annu Rev Pharmacol Toxicol (2018) 58: 625-648]. To further explore the KCNQ channel as a potential therapeutic target of ketamine, it was examined whether pharmacological inhibition of KCNQ alone or in combination with ketamine had any effects on behavior. To accomplish this, a new cohort of mice received two injections with a combination of drugs. Mice were first treated with XE-991, a potent and selective KCNQ (Kcnq2/3) channel inhibitor, using different concentrations (1 and 3 mg/kg BW), in the absence or in combination with (R,S)-ketamine (10 mg/kg BW), and compared to saline-treated controls. To assess the effects of ketamine, XE991 (KCNQ inhibitor), and its combination, these mice were exposed to a FST (FIG. 7A, left). As expected and consistent with the previous findings, a significant decrease in immobility time was found during the FST in mice treated with ketamine, as compared to saline-treated mice (FIG. 23A). No significant differences were found in mice that received XE991 in combination with saline (FIG. 23A), suggesting that XE-991 alone does not produce any acute behavioral effects in the FST. Consistent with the hypothesis, the antidepressant effects of ketamine were abolished in mice that were treated with both XE-991 and ketamine (FIG. 23A). These results indicated that KCNQ activity might be necessary for ketamine to exert its fast-acting antidepressant actions. The inventors then sought to test whether the effects of ketamine could be mimicked, increased or amplified using retigabine, also known as ezogabine, a selective KCNQ (Kcnq2/3) channel activator. Similarly to the previous experiment, mice were treated with saline, (R,S)-ketamine (10 mg/kg BW), or ketamine in combination with two different concentrations of retigabine (1 and 5 mg/kg BW) (FIG. 7A, right). Once again, a significant effect of ketamine as compared to saline controls was found in the FST (FIG. 23B). No effects were found for retigabine when it was administered in combination with saline (FIG. 23B), suggesting that retigabine alone does not produce any acute behavioral effects in the FST at the concentrations tested. However, once administered in combination with ketamine, retigabine (1 mg/kg BW) produced a stronger effect in the FST that was significantly different as compared to ketamine alone (FC: 0.71, pva1=0.0001) (FIGS. 23B and 8B). Interestingly, the combined administration of ketamine, and retigabine at a higher dose (5 mg/kg BW), did not produce any acute behavioral effects in the FST. This is indicative of an inverted U-shaped dose response effect, which has been previously reported for ketamine at higher dosages [N. Li et al., Science (2010) 329: 959-964].

### Example 8

Ketamine and Retigabine Modulate Antidepressant-Like Behaviors in a Semi-Naturalistic Living Environment

To investigate if ketamine treatment modulates antidepressant-like behaviors in mice using an alternative behavioral test, a testing procedure was established that took advantage of a more naturalistic approach for behavioral assessment in rodents, termed the “Social Box” (SB). This system is ideally suited for in-depth investigations of pharmacological manipulations, as it allows for long-term, continuous tracking of social behaviors of groups of mice, in an ethologically-relevant environment, with minimal experimental intervention. Adult male mice living in groups of four were introduced into the SB (FIG. 7D), an enriched housing environment, where they lived under continuous video observation for five days and six nights (FIG. 7E). The first four nights were used to establish individual and group baseline behaviors for all mice. Before the start of the dark phase on Day 5, mice were administered with either (R,S)-ketamine (10 mg/kg/BW) or saline (i.p). All mice were subsequently returned into a clean SB for response monitoring over the following two nights. This procedure was performed on an initial cohort of sixty-four mice (16 groups), allowing assessments of individual differences in ketamine response and establishment of an analysis pipeline for the SB data. A description of the pipeline is discussed above. Briefly, normalized SB behavioral readouts (a total of 306 features) were summarized in three-hourly time bins for the baseline (days 3-4) and response days (days 5-6). To account for baseline individual differences, the change in each readout was calculated for each time bin from the mean of the corresponding time bin over the baseline days. The change values from the first three-hour period of day 5 (following injection) were used to train a supervised partial least squares-based classifier (PLSDA) (FIGS. 7F-G and 24A-D). Based on the PLSDA classifier, mice treated with ketamine spent more time exploring in an open area of the SB arena, using the distal feeder (feeding and drinking away from the nest), exploring in the central labyrinth, approaching others in the group, and engaged in more social behaviors with other members of their group, such as nose to nose contacts. On the other hand, these mice also spent less time around the walls and inside the main nest. These features are all associated with anxiolytic behaviors in mice [Forkosh O. et al., Nat Neurosci (2019) 22: 2023-2028]. A summary of loadings of behavioral readouts onto the PLSDA-based classifier of ketamine response in the SB is provided in FIGS. 24A-B.

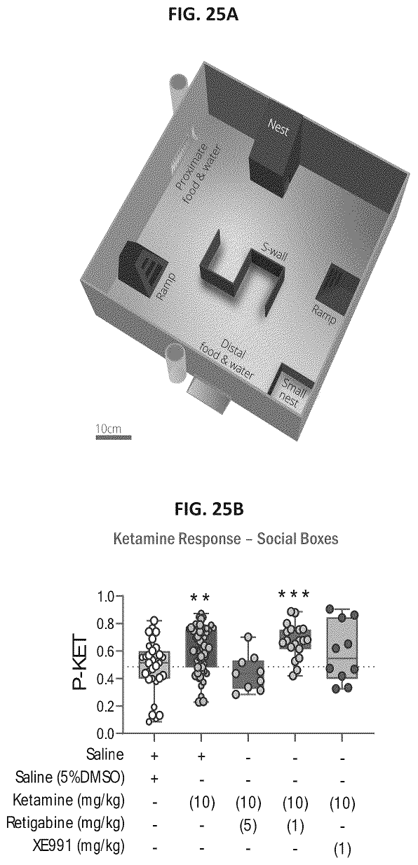

Furthermore, in order to determine whether pharmacological manipulation of KCNQ channels, following ketamine treatment, modulates the behavior of mice in an enriched group environment, a new cohort of ninety-six mice (24 groups), were introduced into the SB. The first four nights were again used to establish individual and group baseline behaviors for all mice. Before the start of the dark phase on Day 5, mice were administered two injections for a combination of either (R,S)-ketamine (10 mg/kg), retigabine (1 or 5 mg/kg) or a saline control (FIG. 7H). An additional group of mice received a combination of ketamine and XE991 (1 mg/kg) (FIGS. 25A-B). The concentrations of retigabine and XE991 were based on the results from the previous experiments using the FST (FIGS. 7C and 23A-B). Two different concentrations of retigabine were investigated in the SB to test whether the inverted u-shaped dose effects of ketamine and retigabine could be reproduced at a higher dose, as observed with FST (FIGS. 7C and 23A-B). Using a prediction algorithm for ketamine response (P-KET), which was developed and trained on the SB data for the original cohort of sixty-four mice (FIGS. 7E-G), the behavior of all mice was monitored after treatment with saline or ketamine alone, or ketamine in combination with KCNQ modulators, for two additional nights (days 5 and 6). Consistent with the previous findings in the FST, a significant difference was uncovered between saline and ketamine-treated mice (FIG. 7I). In addition, mice treated with ketamine in combination with retigabine (1 mg/kg) produced a stronger effect in the SB, as compared to saline-treated mice (FC: 0.72, pva1=0.0005). Similar to the mice from the original cohort, ketamine treated mice from this cohort displayed more social and anxiolytic behaviors. No significant differences were found between mice treated with a combination of ketamine-retigabine (5 mg/kg) or XE991, as compared to saline-treated controls (FIGS. 71, and 25A-B), suggesting, that the combination of ketamine-retigabine at higher doses, or inhibition of KCNQ channels, eliminates the antidepressant-like effects of ketamine in mice. These results are consistent with the previous findings in the FST and suggest that ketamine and pharmacological manipulation of KCNQ channels modulate antidepressant-like behaviors in a semi-naturalistic living environment.

### Example 9

Adjunctive Treatment with Retigabine Augments the Antidepressant-Like Effects of Ketamine but not Escitalopram in Mice

Next, it was examined whether retigabine could increase the sustained antidepressant-like effects produced by a single injection of ketamine in mice. Accordingly, a new cohort of mice were treated with a single dose of saline, ketamine, or ketamine in combination with retigabine (1 mg/kg) and antidepressant-like behaviors were assessed using the FST, 5—and 7-days post-injection (FIG. 8A). Consistent with other reports in the literature, ketamine alone induced a significant decrease in immobility time during the FST that was sustained for several days post-injection (up to five days), however, these effects disappeared by day 7 (FIGS. 8B-C). On the other hand, ketamine in combination with retigabine produced a significant decrease in immobility time during the FST at timepoints tested (days 2, 5 and 7). In addition, the effects produced by the combined treatment of ketamine and retigabine were significantly stronger than the effects of ketamine alone at all timepoints tested (FIGS. 8B-C). Next, as a potential therapeutic strategy for major depressive disorder (MDD), the synergistic effects of ketamine and retigabine was further investigated by testing whether or not retigabine can increase the antidepressant-like effects produced by ketamine in mice. First, mice were treated with (R,S)-ketamine at sub-effective concentrations of 1 and 5 mg/kg/BW, an effective dose of 10 mg/kg/BW, or a saline vehicle control and then their behavior was assessed in the FST (FIG. 8D). Consistent with the previous findings, a significant reduction of immobility time was found in the FST only in mice treated with 10 mg/kg/BW of (R,S)-ketamine, while at lower dosages (1 and 5 mg/kg/BW), ketamine failed to produce antidepressant-like effects (FIG. 8E left, and FIG. 26A).

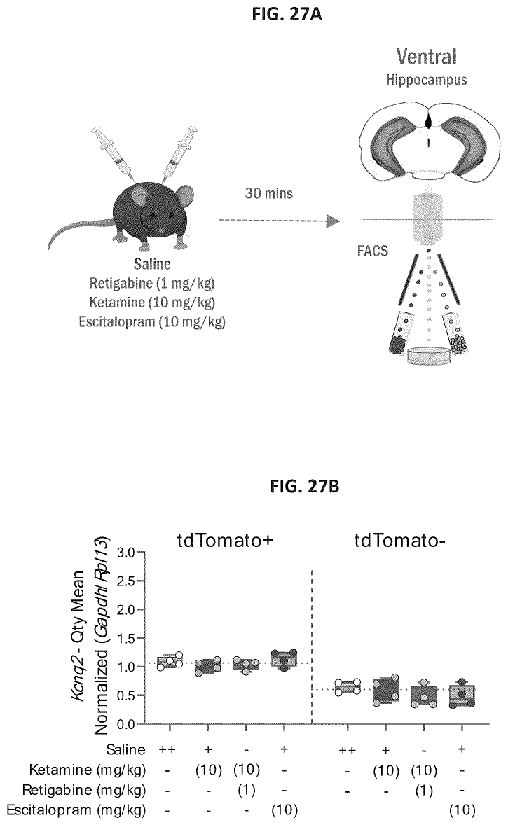

Remarkably, the combined treatment of ketamine with retigabine (1 mg/kg/BW) produced a significant reduction in immobility time during the FST in mice treated with both 5 and 10 mg/kg/BW of ketamine (FIG. 8E right, and FIG. 26A). Next, it was tested whether retigabine can produce similar synergistic effects in combination with traditional antidepressants. Escitalopram, a selective serotonin reuptake inhibitor (SSRI) and commonly prescribed antidepressant in humans that has shown efficacy in preclinical animal (rodent) studies was selected. Interestingly, it was uncovered that retigabine does not increase the antidepressant-like effects produced by escitalopram at any of the concentrations tested (FIG. 26B-C). Lastly, using a new cohort of Nex-Cre-Ai9 mice, it was tested whether the adjunctive treatment of ketamine with retigabine, or escitalopram alone, could modify the expression of Kcnq2 mRNA in FACS sorted cells from the vHipp (FIG. 8F). Consistent with the previous findings, a significant increase of Kcnq2 mRNA was found in glutamatergic neurons (tdTomato+) of the vHipp, two days after treatment (FIG. 8G, left). In addition, it was uncovered that the combined treatment of ketamine with retigabine led to a stronger increase of Kcnq2 mRNA in glutamatergic neurons, as compared to ketamine alone (FIG. 8G, left). No significant changes in Kcnq2 mRNA expression were found after treatment with escitalopram (FIG. 8G, left), or with any of the medications tested in other cell types of the vHipp (tdTomato-) (FIG. 8G, right). Interestingly, it was found that that none of the drugs tested increase the mRNA levels of Kcnq2 in glutamatergic neurons (tdTomato+) or other cell-types (tdTomato-) from the vHipp, 30 minutes after treatment (FIGS. 27A-B). These findings not only complement and verify the results described hereinabove, but suggest that the adjunctive treatment with retigabine augments the antidepressant-like effects of ketamine at later timepoints and lower dosages. Moreover, these results imply that the synergistic effects of retigabine are specific to ketamine but not traditional antidepressants, such as escitalopram and suggest that Kcnq2 may play an important role in the sustained but not the immediate or fast-acting antidepressant effects of ketamine. Taken together, these findings suggest that the adjunctive treatment of ketamine with retigabine increases the sustained antidepressant-like effects of ketamine in mice and highlight the KCNQ channel as a promising target for targeted treatments for major depression.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. A method of treating a psychiatric disorder in a subject in need thereof, the method comprising administering to the subject a therapeutically effective amount of an N-methyl-D-aspartate (NMDA) receptor antagonist and a therapeutically effective amount of a KCNQ channel activator, thereby treating the subject.

2. The method according to claim 1, wherein the NMDA receptor antagonist is selected from the group consisting of ketamine, Traxoprodil (CP-101606), MK-0657, Lanicemine (AZD6765), AVP-786, nitrous oxide, memantine, D-cycloserine (DCS), rapastinel (GLYX-13), and 4-chlorokynurenine (4-Cl-KYNA) (AV-101) or analogs or derivatives thereof.

3. The method according to claim 1, wherein the NMDA receptor antagonist is a ketamine or analogs or derivatives thereof.

4. The method according to claim 3, wherein said therapeutically effective amount of said ketamine comprises a dose of 0.1-1.0 mg/kg body weight for an intravenous or an intramascular route of administration.

5. The method according to claim 3, wherein said therapeutically effective amount of said ketamine comprises a dose of 10-300 mg for an intranasal route of administration.

6. The method according to claim 3, wherein said therapeutically effective amount of said ketamine comprises a dose of 10-500 mg for an oral route of administration.

7. The method according to claim 3, wherein said therapeutically effective amount of said ketamine is lower than the Gold standard administered to psychiatric patients.

8. The method according to claim 1, wherein the KCNQ channel comprises a Kv7.2 subunit.

9. The method according to claim 1, wherein the KCNQ channel activator is selected from the group consisting of retigabine (ezogabine), flupirtine, acrylamide (S)-1, acrylamide (S)-2, BMS-204352, ML213, NS15370, AaTXKp(2-64), diclofenac, meclofenamic acid, meclofenac, NH6, NH29, ICA-27243, ICA-069673, ICA-105665, N-ethylmaleimide, zinc pyrithione and hydrogen peroxide, or analogs or derivatives thereof.

10. The method according to claim 1, wherein the KCNQ channel activator is a retigabine (ezogabine), or analogs or derivatives thereof.

11. The method according to claim 10, wherein said therapeutically effective amount of said retigabine (ezogabine) comprises a dose of 0.5-2000 mg/day.

12. The method according to claim 1, wherein the KCNQ channel activator is ketamine and the KCNQ channel activator is a retigabine (ezogabine).

13. The method according to claim 5, wherein said ketamine is to be administered intranasally.

14. The method according to claim 12, wherein said retigabine (ezogabine) is to be administered orally.

15. The method according to claim 1, wherein the psychiatric disorder is a depression-related disorder.

16. The method according to claim 15, wherein said depression-related disorder is selected from the group consisting of a severe depression, a major depressive disorder (MDD), a treatment-resistant depression, a postpartum depression and a psychotic depression.

17. The method according to claim 1, wherein the psychiatric disorder is selected from the group consisting of a bipolar disorder, a schizophrenia, a neuropathic pain, a post-traumatic stress disorder (PTSD), an obsessive-compulsive disorder (OCD), a pervasive developmental disorder (PDD), a post-traumatic stress disorder (PTSD), a panic attack, an anxiety disorder, a social phobia, a sleep disorder, an eating disorder, a stress, a fatigue, a chronic pain and a substance-related disorder.

18. The method according to claim 1, wherein the subject is a human being.


SEQUENCE LISTING (39 sequences)

SEQ ID NO: 1 - DNA, 20 bp, synthetic construct
  misc_feature 1..20: note = Single strand DNA oligonucleotide
  source 1..20: mol_type = other DNA
gtgcacctgaacctcaacct

SEQ ID NO: 2 - DNA, 20 bp, synthetic construct
  misc_feature 1..20: note = Single strand DNA oligonucleotide
  source 1..20: mol_type = other DNA
cgggaaacagtaggaaacca

SEQ ID NO: 3 - DNA, 20 bp, synthetic construct
  misc_feature 1..20: note = Single strand DNA oligonucleotide
  source 1..20: mol_type = other DNA
acagctctttgcgtggagtt

SEQ ID NO: 4 - DNA, 20 bp, synthetic construct
  misc_feature 1..20: note = Single strand DNA oligonucleotide
  source 1..20: mol_type = other DNA
gccatcctgcctccttctat

SEQ ID NO: 5 - DNA, 20 bp, synthetic construct
  misc_feature 1..20: note = Single strand DNA oligonucleotide
  source 1..20: mol_type = other DNA
ctggaccacaacatcgtgac

SEQ ID NO: 6 - DNA, 20 bp, synthetic construct
  misc_feature 1..20: note = Single strand DNA oligonucleotide
  source 1..20: mol_type = other DNA
gccggtcaaggtaacaaaga

SEQ ID NO: 7 - DNA, 20 bp, synthetic construct
  misc_feature 1..20: note = Single strand DNA oligonucleotide
  source 1..20: mol_type = other DNA
caactgcaggagagtgagca

SEQ ID NO: 8 - DNA, 20 bp, synthetic construct
  misc_feature 1..20: note = Single strand DNA oligonucleotide
  source 1..20: mol_type = other DNA
ccatctgttctgcagggtct

SEQ ID NO: 9 - DNA, 24 bp, synthetic construct
  misc_feature 1..24: note = Single strand DNA oligonucleotide
  source 1..24: mol_type = other DNA
ccatcaccatcttccaggagcgag

SEQ ID NO: 10 - DNA, 24 bp, synthetic construct
  misc_feature 1..24: note = Single strand DNA oligonucleotide
  source 1..24: mol_type = other DNA
gatggcatggactgtggtcatgag

SEQ ID NO: 11 - DNA, 20 bp, synthetic construct
  misc_feature 1..20: note = Single strand DNA oligonucleotide
  source 1..20: mol_type = other DNA
gcatgaaggagacccagaag

SEQ ID NO: 12 - DNA, 20 bp, synthetic construct
  misc_feature 1..20: note = Single strand DNA oligonucleotide
  source 1..20: mol_type = other DNA
cactgagaccaggctcttcc

SEQ ID NO: 13 - DNA, 20 bp, synthetic construct
  misc_feature 1..20: note = Single strand DNA oligonucleotide
  source 1..20: mol_type = other DNA
atgttgctttcacggacaca

SEQ ID NO: 14 - DNA, 20 bp, synthetic construct
  misc_feature 1..20: note = Single strand DNA oligonucleotide
  source 1..20: mol_type = other DNA
gggccagtgcttcatatcag

SEQ ID NO: 15 - DNA, 21 bp, synthetic construct
  misc_feature 1..21: note = Single strand DNA oligonucleotide
  source 1..21: mol_type = other DNA
gctcttctgatgctacggtga

SEQ ID NO: 16 - DNA, 21 bp, synthetic construct
  misc_feature 1..21: note = Single strand DNA oligonucleotide
  source 1..21: mol_type = other DNA
gagaagcgttctcttcaaacc

SEQ ID NO: 17 - DNA, 20 bp, synthetic construct
  misc_feature 1..20: note = Single strand DNA oligonucleotide
  source 1..20: mol_type = other DNA
taccgcaagctgcagaattt

SEQ ID NO: 18 - DNA, 20 bp, synthetic construct
  misc_feature 1..20: note = Single strand DNA oligonucleotide
  source 1..20: mol_type = other DNA
cccctcagagctcttctcgt

SEQ ID NO: 19 - DNA, 20 bp, synthetic construct
  misc_feature 1..20: note = Single strand DNA oligonucleotide
  source 1..20: mol_type = other DNA
tgcctggtacataggcttcc

SEQ ID NO: 20 - DNA, 20 bp, synthetic construct
  misc_feature 1..20: note = Single strand DNA oligonucleotide
  source 1..20: mol_type = other DNA
agacgtccttcccaggtttt

SEQ ID NO: 21 - DNA, 20 bp, synthetic construct
  misc_feature 1..20: note = Single strand DNA oligonucleotide
  source 1..20: mol_type = other DNA
gcaggtctctgtaggccttg

SEQ ID NO: 22 - DNA, 20 bp, synthetic construct
  misc_feature 1..20: note = Single strand DNA oligonucleotide
  source 1..20: mol_type = other DNA
gtgcagccagttgtagcaga

SEQ ID NO: 23 - DNA, 20 bp, synthetic construct
  misc_feature 1..20: note = Single strand DNA oligonucleotide
  source 1..20: mol_type = other DNA
aggaggtcctgggtgacttt

SEQ ID NO: 24 - DNA, 20 bp, synthetic construct
  misc_feature 1..20: note = Single strand DNA oligonucleotide
  source 1..20: mol_type = other DNA
cagtaccccctgctccaata

SEQ ID NO: 25 - DNA, 20 bp, synthetic construct
  misc_feature 1..20: note = Single strand DNA oligonucleotide
  source 1..20: mol_type = other DNA
tggaaagggtcaagttcagg

SEQ ID NO: 26 - DNA, 20 bp, synthetic construct
  misc_feature 1..20: note = Single strand DNA oligonucleotide
  source 1..20: mol_type = other DNA
ggtctcttgccaatcctcag

SEQ ID NO: 27 - DNA, 23 bp, synthetic construct
  misc_feature 1..23: note = Single strand DNA oligonucleotide
  source 1..23: mol_type = other DNA
cactctggaggagaaacggaagg

SEQ ID NO: 28 - DNA, 21 bp, synthetic construct
  misc_feature 1..21: note = Single strand DNA oligonucleotide
  source 1..21: mol_type = other DNA
gcaggcatgaggcaaacagtc

SEQ ID NO: 29 - DNA, 20 bp, synthetic construct
  misc_feature 1..20: note = Single strand DNA oligonucleotide
  source 1..20: mol_type = other DNA
ctggggtccttgtgcagtat

SEQ ID NO: 30 - DNA, 20 bp, synthetic construct
  misc_feature 1..20: note = Single strand DNA oligonucleotide
  source 1..20: mol_type = other DNA
aacagggttcatgagcttgg

SEQ ID NO: 31 - DNA, 20 bp, synthetic construct
  misc_feature 1..20: note = Single strand DNA oligonucleotide
  source 1..20: mol_type = other DNA
ctggaacgtgacaaatgcca

SEQ ID NO: 32 - DNA, 20 bp, synthetic construct
  misc_feature 1..20: note = Single strand DNA oligonucleotide
  source 1..20: mol_type = other DNA
cggcgaagatgatgaggaac

SEQ ID NO: 33 - DNA, 20 bp, synthetic construct
  misc_feature 1..20: note = Single strand DNA oligonucleotide
  source 1..20: mol_type = other DNA
aaaaagcctggggcaataat

SEQ ID NO: 34 - DNA, 20 bp, synthetic construct
  misc_feature 1..20: note = Single strand DNA oligonucleotide
  source 1..20: mol_type = other DNA
ctcacctgctccaggttctc

SEQ ID NO: 35 - DNA, 20 bp, synthetic construct
  misc_feature 1..20: note = Single strand DNA oligonucleotide
  source 1..20: mol_type = other DNA
ggcattaaagcagcgtatcc

SEQ ID NO: 36 - DNA, 19 bp, synthetic construct
  misc_feature 1..19: note = Single strand DNA oligonucleotide
  source 1..19: mol_type = other DNA
ctgttcctgtacggcatgg

SEQ ID NO: 37 - RNA, 72 nt, synthetic construct
  source 1..72: mol_type = other RNA; note = shRNA
ggtaccgatcccactaccgttgttataggtgttcaagagacacctataacaacggtagtttttttgggatcc

SEQ ID NO: 38 - RNA, 72 nt, synthetic construct
  source 1..72: mol_type = other RNA; note = shRNA
ggtaccgatcccggtattcggtgttgagtacttcaagagagtactcaacaccgaataccttttttgggatcc

SEQ ID NO: 39 - RNA, 75 nt, synthetic construct
  source 1..75: mol_type = other RNA; note = shRNA
ggtaccgatccccgtggtattcggtgttgagtaccaagaggtactcaacaccgaataccacgttttttgggatcc